(19) United States
(12) Patent Application Publication
(10) Pub. No.: US 2024/0368084 A1
(43) Pub. Date: Nov. 7, 2024

(54) STING MODULATORS, COMPOSITIONS, AND METHODS OF USE

(71) Applicant: University of South Florida, Tampa, FL (US)

(72) Inventors: Wayne Charles GUIDA, Saint Pete Beach, FL (US); James William LEAHY, Lutz, FL (US); Jamie Marie NUNZIATA, Tampa, FL (US)

(21) Appl. No.: 18/687,558

(22) PCT Filed: Sep. 2, 2022
(86) PCT No.: PCT/US2022/042493

§ 371 Date: Feb. 28, 2024

Related U.S. Application Data
(60) Provisional application No. 63/240,462, filed on Sep. 3, 2021.

(51) Int. Cl. C07D 213/74; A61K 31/444

(52) U.S. Cl. CPC C07D 213/74; A61K 31/444

(57) ABSTRACT

The present disclosure is directed to compounds of Formula (I), or pharmaceutically acceptable salts thereof and compounds of Formula (II), or pharmaceutically acceptable salts thereof, that modulate stimulator of interferon genes (STING), compositions comprising such compounds, and methods of using same for the treatment of disorders such as cancer and autoimmune disease.

### CROSS-REFERENCE TO RELATED APPLICATIONS

This application claims priority to U.S. Provisional Application No. 63/240,462, filed on Sep. 3, 2021, which is incorporated by reference herein in its entirety.

### FEDERALLY SPONSORED RESEARCH OR DEVELOPMENT

This invention was made with government support under Grant No. R2AI149450, awarded by the National Institutes of Health. The government has certain rights in the invention.

### BACKGROUND

Cancer is one of the world's most dreaded diseases. Tumor cells are hard to eliminate due their aberrant genetics, which results in uncontrolled growth. For example, “cold tumors” are a type of tumor that is not recognized and eradicated by the immune system. The STING (STimulator of INterferon Genes) pathway is involved in the innate immune response, which can help combat cancer, as well as cause certain autoimmune disorders such as systemic lupus erythematosus (SLE) and other diseases that are associated with an accumulation of nucleic acids in the cytoplasm.

STING-mediated production of IFN-β within the tumor microenvironment can result in activation of tumor antigen-specific CD8⁺ T-cell immunity that can lead to tumor regression.

Mechanistic studies have shown that STING induced anti-tumor immunity is likely due to a pro-inflammatory cytokine response as well as the tumor specific CD8⁺ T-cellular response.

STING activation by STING agonists should result in innate T-cell mediated anti-tumor immunity in the tumor microenvironment and have significant potential as a therapeutic strategy for the treatment of patients with advanced solid tumors. On the other hand, inhibition of STING (by STING antagonists) would lead to a decreased production of IFN-β and other Interferon Stimulated Genes (ISG) which are comprised of approximately 300 cytokines induced by the transcription factors interferon regulatory factor 3 (IRF3) and nuclear factor kappa-light-chain-enhancer of activated B cells (NF-κB). Inhibiting STING could have implications in the treatment of autoimmune disease such as lupus erythematosus.

Recently, there has been interest in developing agonists to increase activity of the STING pathway as a modality for cancer treatment. Most STING agonists developed to date have been cyclic di-nucleotides (CDNs). In contrast, rather than activating STING to provoke an immune anti-tumor response, STING antagonists reduce anti-DNA antibody production resulting from abnormal removal of cytoplasmic DNA in immune cells. This leads to the accumulation of autoantibodies, chronic inflammation, and organ dysfunction that are hallmarks of SLE. In addition to SLE, accumulation of abnormal levels of cytoplasmic or lysosomal DNA (leading to STING activation) relate to several other diseases, including viral infections.

### SUMMARY

Some embodiments provide a compound of Formula (I), or a pharmaceutically acceptable salt thereof:

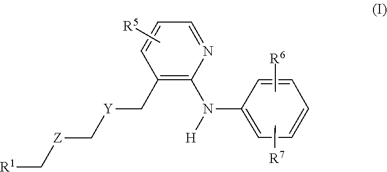

wherein Y, Y¹, Z, R¹, R², R³, R^{3A}, R⁴, R^{4A}, R⁶, R⁷, R⁸, R⁹, R¹⁰, and R′, are as defined herein.

Some embodiments, provide compounds of Formula (II), or pharmaceutically acceptable salts thereof.

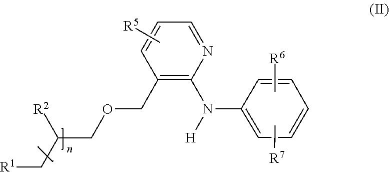

wherein n, R¹, R², R³, R^{3A}, R⁴, R⁵, R⁶, R⁷, R⁸, R⁹, R¹⁰, and R′, are as defined herein.

Some embodiments provide a method of treating cancer comprising administering a therapeutically effective amount of a compound of Formula (I), or a pharmaceutically acceptable salt thereof, to a subject in need thereof. Some embodiments provide a method of treating cancer comprising administering a therapeutically effective amount of a compound of Formula (II), or a pharmaceutically acceptable salt thereof, to a subject in need thereof.

Some embodiments provide a method of treating an autoimmune disorder comprising administering a therapeutically effective amount of a compound of Formula (I), or a pharmaceutically acceptable salt thereof, to a subject in need thereof. Some embodiments provide a method of treating an autoimmune disorder comprising administering a therapeutically effective amount of a compound of Formula (II), or a pharmaceutically acceptable salt thereof, to a subject in need thereof.

### DESCRIPTION OF DRAWINGS

FIG. 1 depicts a plot showing the results of a microscale thermophoresis (MST) binding assay on compound 1.

FIG. 2 depicts a plot showing the results of a microscale thermophoresis (MST) binding assay on compound 4.

FIG. 3 depicts a plot showing the results of a microscale thermophoresis (MST) binding assay on compound 48.

FIG. 4 depicts a plot showing the results of a microscale thermophoresis (MST) binding assay on compound 49.

FIG. 5 depicts a plot showing the results of a microscale thermophoresis (MST) binding assay on compound 8.

FIG. 6 depicts a plot showing the results of a microscale thermophoresis (MST) binding assay on compound 9.

FIG. 7 depicts a plot showing the results of a microscale thermophoresis (MST) binding assay on compound 10.

FIG. 8 depicts a plot showing the results of a microscale thermophoresis (MST) binding assay on compound 11.

FIG. 9 depicts a plot showing the results of a microscale thermophoresis (MST) binding assay on compound 13.

FIG. 10 depicts a plot showing the results of a microscale thermophoresis (MST) binding assay on compound 14.

FIG. 11 depicts a plot showing the results of a microscale thermophoresis (MST) binding assay on compound 15.

FIG. 12 depicts a plot showing the results of a microscale thermophoresis (MST) binding assay on compound 16.

FIG. 13 depicts a plot showing the results of a microscale thermophoresis (MST) binding assay on compound 17.

FIG. 14 depicts a plot showing the results of a microscale thermophoresis (MST) binding assay on compound 19.

FIG. 15 depicts a plot showing the results of a microscale thermophoresis (MST) binding assay on compound 21.

FIG. 16 depicts a plot showing the results of a microscale thermophoresis (MST) binding assay on compound 23.

FIG. 17 depicts a plot showing the results of a microscale thermophoresis (MST) binding assay on compound 24.

FIG. 18 depicts a plot showing the results of a microscale thermophoresis (MST) binding assay on compound 26.

FIG. 19 depicts a plot showing the results of a microscale thermophoresis (MST) binding assay on compound 27.

FIG. 20 depicts a plot showing the results of a microscale thermophoresis (MST) binding assay on compound 29.

FIG. 21 depicts a plot showing the results of a microscale thermophoresis (MST) binding assay on compound 30.

FIG. 22 depicts a plot showing the results of a microscale thermophoresis (MST) binding assay on compound 32.

FIG. 23 depicts a plot showing the results of a microscale thermophoresis (MST) binding assay on compound 33.

FIG. 24 depicts a plot showing the results of a microscale thermophoresis (MST) binding assay on compound 35.

FIG. 25 depicts a plot showing the results of a microscale thermophoresis (MST) binding assay on compound 36.

FIG. 26 depicts a plot showing the results of a microscale thermophoresis (MST) binding assay on compound 38.

FIG. 27 depicts a plot showing the results of a microscale thermophoresis (MST) binding assay on compound 39.

FIG. 28 depicts a plot showing the results of a microscale thermophoresis (MST) binding assay on compound 40.

FIG. 29 depicts a plot showing the results of a microscale thermophoresis (MST) binding assay on compound 41.

### DETAILED DESCRIPTION

Some embodiments, provide compounds of Formula (I),

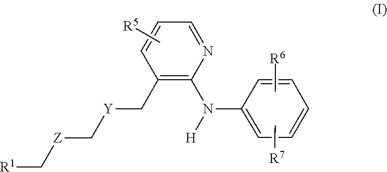

wherein:
   Y and Y¹ are independently selected from O, NR^{4A}, CH₂, or absent;
   Z is independently selected from C1-C10 alkylene, C2-C10 alkenylene, C2-C10 alkynylene, or 3-10 membered heteroalkylene, each optionally substituted with 1-3 independently selected R²;
   R¹ and each occurrence of R² are independently selected from hydrogen, OH, OR³,
   Y¹R^{3A}, SR³, and NR³R⁴;
   R³, R⁴, and R^{4A} are independently selected from hydrogen, C1-C10 alkyl optionally substituted with 1-6 halogens, C6-C10 aryl, and 5 to 10 membered heteroaryl; or
   R³ and R⁴, together with the nitrogen atom to which they are attached, can come together to form a 3 to 7 membered heterocyclyl or 5 to 10 membered heteroaryl;
   R^{3A} is

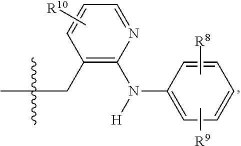

wherein [symbol] represents the point of connection of R^{3A} to the remainder of the molecule;
R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl;
each occurrence of R′ is independently selected from H and C1-C6 alkyl.

In some embodiments, R¹ and each occurrence of R² collectively comprise 0-1 occurrences of Y¹R^{3A}. In some embodiments, when one occurrence of R¹ and R² is Y¹R^{3A}, the other occurrences of R¹ and R² are not Y¹R^{3A}.

In some embodiments, Y and Y¹ are independently selected from O and NR^{4A}. In some embodiments, Y is O. In other embodiments, Y is NR^{4A}. For example, Y is NH. In still other embodiments, Y is CH₂. In some embodiments, Y is absent. In some embodiments, Y¹ is 0. In other embodiments, Y¹ is NR^{4A}. For example, Y¹ is NH. In still other embodiments, Y¹ is CH₂. In some embodiments, Y¹ is absent.

In some embodiments, Z is C1-C10 alkylene optionally substituted with 1-3 independently selected R². In some embodiments, Z is C1-C10 alkylene substituted with 1-3 independently selected R². In some embodiments, Z is unsubstituted C1-C10 alkylene. In some embodiments, the Z C1-C10 alkylene is a methylene. In some embodiments, the Z C1-C10 alkylene is an ethylene. In some embodiments, the Z C1-C10 alkylene is a C3 alkylene (e.g., n-propylene). In some embodiments, the Z C1-C10 alkylene is a C4 alkylene (e.g., n-butylene). In some embodiments, the Z C1-C10 alkylene is a C5 alkylene (e.g., n-pentylene). In some embodiments, the Z C1-C10 alkylene is a C6-C10 alkylene. In some embodiments, Z is methylene substituted with OH. In some embodiments, Z is ethylene substituted with two OH.

In some embodiments, Z is C2-C10 alkenylene optionally substituted with 1-3 independently selected R². In some embodiments, Z is C2-C10 alkenylene substituted with 1-3 independently selected R². In some embodiments, Z is unsubstituted C2-C10 alkenylene. In some embodiments, the Z C2-C10 alkenylene is an ethenylene. In some embodiments, the Z C2-C10 alkenylene is a C3 alkenylene. In some embodiments, the Z C2-C10 alkenylene is a C4 alkenylene. In some embodiments, the Z C2-C10 alkenylene is a C5 alkenylene. In some embodiments, the Z C2-C10 alkenylene is a C6-C10 alkenylene.

In some embodiments, Z is C2-C10 alkynylene optionally substituted with 1-3 independently selected R². In some embodiments, Z is C2-C10 alkynylene substituted with 1-3 independently selected R². In some embodiments, Z is unsubstituted C2-C10 alkynylene. In some embodiments, the Z C2-C10 alkynylene is an ethynylene. In some embodiments, the Z C2-C10 alkynylene is a C3 alkynylene. In some embodiments, the Z C2-C10 alkynylene is a C4 alkynylene. In some embodiments, the Z C2-C10 alkynylene is a C5 alkynylene. In some embodiments, the Z C2-C10 alkynylene is a C6-C10 alkynylene.

In some embodiments, Z is 3-10 membered heteroalkylene optionally substituted with 1-3 independently selected R². In some embodiments, Z is 3-10 membered heteroalkylene substituted with 1-3 independently selected R². In some embodiments, Z is unsubstituted 3-10 membered heteroalkylene. In some embodiments, the Z 3-10 membered heteroalkylene is a C3 heteroalkylene. In some embodiments, the Z 3-10 membered heteroalkylene is a C4 heteroalkylene. In some embodiments, the Z 3-10 membered heteroalkylene is a C5 heteroalkylene. In some embodiments, the Z 3-10 membered heteroalkylene is a C6-C10 heteroalkylene. In some embodiments, the Z 3-10 membered heteroalkylene comprises one or two O atoms (e.g., one O atom). In some embodiments, the Z 3-10 membered heteroalkylene comprises one S atom. In some embodiments, the Z 3-10 membered heteroalkylene comprises one NH.

In some embodiments, R¹ is hydrogen. In some embodiments, R¹ is OH. In some embodiments, R¹ is OR³. In some embodiments, R¹ is Y¹R^{3A}. In certain embodiments, R¹ is R^{3A}. In some embodiments, R¹ is SR³. In some embodiments, R¹ is NR³R⁴. In some embodiments, R¹ is selected from OH, OR³, Y¹R^{3A}, SR³, and NR³R⁴. In some embodiments, R¹ is NR³R⁴. In some embodiments, R¹ is selected from OH and Y¹R^{3A}.

In some embodiments, Z is unsubstituted. In some embodiments, Z is substituted with one R². In some embodiments, Z is substituted with two independently selected R². In some embodiments, Z is substituted with three independently selected R². In some embodiments, at least one R² is hydrogen. In some embodiments, at least one R² is OH. In some embodiments, each R² is OH. In some embodiments, at least one R² is OR³. In some embodiments, at least one R² is Y¹R^{3A}. In certain embodiments, at least one R² is R^{3A}. In some embodiments, at least one R² is SR³. In some embodiments, at least one R² is NR³R⁴. In some embodiments, each R² is independently selected from OH, OR³, Y¹R^{3A}, SR³, and NR³R⁴. In some embodiments, each R² is independently selected from OH and Y¹R^{3A}.

In some embodiments, R¹ and each occurrence of R² are independently selected from OH, OR³, and Y¹R^{3A}. In some embodiments, R¹ is Y¹R^{3A} and each occurrence of R² is selected from OH and OR³. In some embodiments, R¹ and each occurrence of R² are each OH. In some embodiments, R¹ and R² are independently selected from hydrogen, OH, OR³, Y¹R^{3A}, SR³, and NR³R⁴. In some embodiments, R¹ is Y¹R^{3A} and R² is selected from hydrogen, OH, OR³, SR³, and NR³R⁴.

In some embodiments, R^{4A} is hydrogen.

In some embodiments, R^{4A} is C1-C10 alkyl optionally substituted with 1-6 halogens. In some embodiments, R^{4A} is C1-C10 alkyl optionally substituted with 1-3 halogens. In some embodiments, R^{4A} is C1-C10 alkyl optionally substituted with 1-3 fluoro. In some embodiments, R^{4A} is C1-C6 alkyl optionally substituted with 1-3 halogens. In some embodiments, R^{4A} is C1-C3 alkyl optionally substituted with 1-3 halogens. In some embodiments, R^{4A} is C1-C6 alkyl optionally substituted with 1-3 fluoro.

In some embodiments, R^{4A} is an unsubstituted C1-C6 alkyl. In some embodiments, R^{4A} is an unsubstituted C1-C3 alkyl. In some embodiments, R^{4A} is methyl.

In some embodiments, R^{4A} is C1-C10 alkyl substituted with 1-6 halogens. In some embodiments, R^{4A} is C1-10 alkyl substituted with 1-3 halogens. In some embodiments, R^{4A} is C1-C10 alkyl substituted with 1-3 fluoro. In some embodiments, R^{4A} is C1-C6 alkyl substituted with 1-3 halogens. In some embodiments, R^{4A} is C1-C3 alkyl substituted with 1-3 halogens. In some embodiments, R^{4A} is C1-C6 alkyl substituted with 1-3 fluoro. In some embodiments, R^{4A} is trifluoromethyl.

In some embodiments, R^{4A} is C6-C10 aryl, such as phenyl or naphthyl. In some embodiments, R^{4A} is 5 to 10 membered heteroaryl, such as pyrrole, imidazole, pyridine, pyrimidine, or quinoline.

In some embodiments, R³ is hydrogen. In some embodiments, R³ is C1-C10 alkyl optionally substituted with 1-6 halogens. In some embodiments, R³ is an unsubstituted C1-C10 alkyl. In some embodiments, R³ is an unsubstituted C1-C6 alkyl. In some embodiments, R³ is methyl.

In some embodiments, R³ is C1-C6 alkyl optionally substituted with 1-3 halogens. In some embodiments, each halogen is fluoro. In some embodiments, R³ is trifluoromethyl. In some embodiments, R³ is C6-C10 aryl, such as phenyl or napthyl. In some embodiments, R³ is 5 to 10 membered heteroaryl, such as pyrrole, imidazole, pyridine, pyrimidine, or quinoline.

In some embodiments, R⁴ is hydrogen. In some embodiments, R⁴ is C1-C10 alkyl optionally substituted with 1-6 halogens. In some embodiments, R⁴ is methyl. In some embodiments, R⁴ is trifluoromethyl. In some embodiments, R⁴ is C6-C10 aryl, such as phenyl or napthyl. In some embodiments, R⁴ is 5 to 10 membered heteroaryl, such as pyrrole, imidazole, pyridine, pyrimidine, or quinoline.

In some embodiments, R³ and R⁴, together with the nitrogen atom to which they are attached, come together to form a 3 to 7 membered heterocyclyl. In some embodiments, R³ and R⁴, together with the nitrogen atom to which they are attached, come together to form a 5 to 10 membered heteroaryl.

In some embodiments, Z is methylene substituted with OH and R¹ is OH. In some embodiments, when Z is C1-C10 alkylene optionally substituted with 1-3 R² and one of R¹ and each occurrence of R² is OH, the other of R¹ and each occurrence of R² are not both OH. In some embodiments, when Z is C1-C10 alkylene substituted with one R², R¹ and R² are both OH. In some embodiments, when Y is O, then R¹ and R² are not both OH. In some embodiments, when Y is O, then R¹ and R² are both OH. In some embodiments, when R⁵ is hydrogen and Z is C1-C10 alkylene optionally substituted with 1-3 R², R¹ and R² are not both OH. In some embodiments, when Z is C1-C10 alkylene optionally substituted with 1-3 R², y is O, and R⁵ is hydrogen, R¹ and R² are not both OH.

In some embodiments, compounds of Formula (I), or pharmaceutically acceptable salts thereof, function as STING agonists. In other embodiments, compounds of Formula (I), or pharmaceutically acceptable salts thereof, function as partial STING agonists. In still other embodiments, compounds of Formula (I), or pharmaceutically acceptable salts thereof, function as partial or full STING antagonists.

Some embodiments provide compounds of Formula (II), or pharmaceutically acceptable salts thereof,

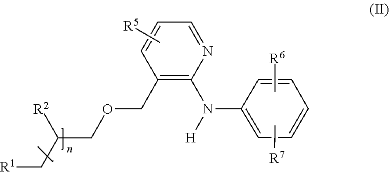

wherein:
   R¹ is selected from OH, OR³, SR³, NR³R⁴, and

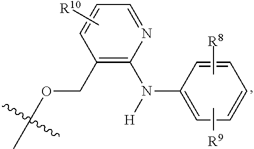

wherein [symbol] represents the point of connection of R¹ to the remainder of the molecule;
n is 0, 1, 2, or 3;
each occurrence of R² is an independently selected hydrogen, OH, OR³, SR³, or NR³R⁴;
R³ and R⁴ are independently selected from hydrogen, C1-C10 alkyl optionally substituted with 1-6 halogens, C6-C10 aryl, and 5 to 10 membered heteroaryl; or
R³ and R⁴, together with the nitrogen atom to which they are attached, can come together to form a 3 to 7 membered heterocyclyl or 5 to 10 membered heteroaryl; and
R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl; and
each occurrence of R′ is independently selected from H and C1-C6 alkyl.

In some embodiments, n is O. In some embodiments, n is 1. In some embodiments, n is 2. In some embodiments, n is 3.

In some embodiments, R¹ is OH. In some embodiments, R¹ is OR³. In some embodiments, R¹ is SR³. In some embodiments, R¹ is NR³R⁴. In some embodiments, R¹ is

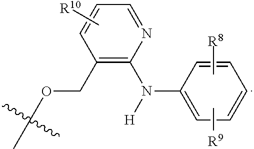

In some embodiments, R¹ is selected from OH and

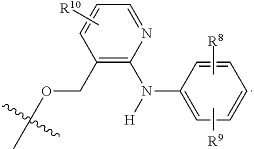

In some embodiments, R² is hydrogen. In some embodiments, R² is OH. In some embodiments, R² is OR³. In some embodiments, R² is SR³. In some embodiments, R² is NR³R⁴.

In some embodiments, R³ is hydrogen. In some embodiments, R³ is C1-C10 alkyl optionally substituted with 1-6 halogens. In some embodiments, R³ is methyl. In some embodiments, R³ is trifluoromethyl. In some embodiments, R³ is C6-C10 aryl, such as phenyl or napthyl. In some embodiments, R³ is 5 to 10 membered heteroaryl.

In some embodiments, R⁴ is hydrogen. In some embodiments, R⁴ is C1-C10 alkyl optionally substituted with 1-6 halogens. In some embodiments, R⁴ is methyl. In some embodiments, R⁴ is trifluoromethyl. In some embodiments, R⁴ is C6-C10 aryl. In some embodiments, R⁴ is 5 to 10 membered heteroaryl.

In some embodiments, R³ and R⁴, together with the nitrogen atom to which they are attached, come together to form a 3 to 7 membered heterocyclyl. In some embodiments, R³ and R⁴, together with the nitrogen atom to which they are attached, come together to form a 5 to 10 membered heteroaryl.

In some embodiments, R⁵ is halogen. In some embodiments, R⁵ is fluoro or chloro. In some embodiments, R⁵ is pseudohalogen.

In some embodiments, R⁵ is C1-C10 alkyl optionally substituted with 1-6 halogens. In some embodiments, R⁵ is C1-C6 alkyl optionally substituted with 1-6 halogens. In some embodiments, R⁵ is C1-C6 alkyl optionally substituted with 1-3 halogens. In some embodiments, R⁵ is C1-C3 alkyl optionally substituted with 1-3 halogens. In some embodiments, R⁵ is C1-C6 alkyl substituted with 1-6 halogens. In some embodiments, R⁵ is C1-C6 alkyl substituted with 1-3 halogens. In some embodiments, R⁵ is C1-C3 alkyl substituted with 1-3 halogens. In some embodiments, R⁵ is an unsubstituted C1-C10 alkyl. In some embodiments, R⁵ is an unsubstituted C1-C6 alkyl. In some embodiments, R⁵ is an unsubstituted C1-C3 alkyl. In some embodiments, R⁵ is methyl. In some embodiments, R⁵ is trifluoromethyl.

In some embodiments, R⁵ is C6-C10 aryl. In some embodiments, R⁵ is 5 to 10 membered heteroaryl.

In some embodiments, R⁵ is C1-C6 alkoxy. For example, R⁵ is methoxy. In some embodiments, R⁵ is cyano. In some embodiments, R⁵ is nitro. In some embodiments, R⁵ is C(O)C1-C6 alkyl. For example, R⁵ is C(O)Me. In some embodiments, R⁵ is CO₂C1-C6 alkyl. For example, R⁵ is CO₂Me. In some embodiments, R⁵ is C(O)NHC1-C6 alkyl. For example, R⁵ is C(O)NHMe. In some embodiments, R⁵ is N(R′)₂. For example, R⁵ is NH₂, NHMe, or NMe₂.

In some embodiments, R⁶ is halogen. In some embodiments, R⁶ is fluoro or chloro. In some embodiments, R⁶ is pseudohalogen.

In some embodiments, R⁶ is C1-C10 alkyl optionally substituted with 1-6 halogens. In some embodiments, R⁶ is C1-C6 alkyl optionally substituted with 1-6 halogens. In some embodiments, R⁶ is C1-C6 alkyl optionally substituted with 1-3 halogens. In some embodiments, R⁶ is C1-C3 alkyl optionally substituted with 1-3 halogens. In some embodiments, R⁶ is C1-C6 alkyl substituted with 1-6 halogens. In some embodiments, R⁶ is C1-C6 alkyl substituted with 1-3 halogens. In some embodiments, R⁶ is C1-C3 alkyl substituted with 1-3 halogens. In some embodiments, R⁶ is an unsubstituted C1-C10 alkyl. In some embodiments, R⁶ is an unsubstituted C1-C6 alkyl. In some embodiments, R⁶ is an unsubstituted C1-C3 alkyl. In some embodiments, R⁶ is methyl. In some embodiments, R⁶ is trifluoromethyl.

In some embodiments, R⁶ is C6-C10 aryl. In some embodiments, R⁶ is 5 to 10 membered heteroaryl.

In some embodiments, R⁶ is C1-C6 alkoxy. For example, R⁶ is methoxy. In some embodiments, R⁶ is cyano. In some embodiments, R⁶ is nitro. In some embodiments, R⁶ is C(O)C1-C6 alkyl. For example, R⁶ is C(O)Me. In some embodiments, R⁶ is CO₂C1-C6 alkyl. For example, R⁶ is CO₂Me. In some embodiments, R⁶ is C(O)NHC1-C6 alkyl. For example, R⁶ is C(O)NHMe. In some embodiments, R⁶ is N(R′)₂. For example, R⁶ is NH₂, NHMe, or NMe₂.

In some embodiments, R⁷ is halogen. In some embodiments, R⁷ is fluoro or chloro. In some embodiments, R⁷ is pseudohalogen.

In some embodiments, R⁷ is C1-C10 alkyl optionally substituted with 1-6 halogens. In some embodiments, R⁷ is C1-C6 alkyl optionally substituted with 1-6 halogens. In some embodiments, R⁷ is C1-C6 alkyl optionally substituted with 1-3 halogens. In some embodiments, R⁷ is C1-C3 alkyl optionally substituted with 1-3 halogens. In some embodiments, R⁷ is C1-C6 alkyl substituted with 1-6 halogens. In some embodiments, R⁷ is C1-C6 alkyl substituted with 1-3 halogens. In some embodiments, R⁷ is C1-C3 alkyl substituted with 1-3 halogens. In some embodiments, R⁷ is an unsubstituted C1-C10 alkyl. In some embodiments, R⁷ is an unsubstituted C1-C6 alkyl. In some embodiments, R⁷ is an unsubstituted C1-C3 alkyl. In some embodiments, R⁷ is methyl. In some embodiments, R⁷ is trifluoromethyl.

In some embodiments, R⁷ is C6-C10 aryl. In some embodiments, R⁷ is 5 to 10 membered heteroaryl.

In some embodiments, R⁷ is C1-C6 alkoxy. For example, R⁷ is methoxy. In some embodiments, R⁷ is cyano. In some embodiments, R⁷ is nitro. In some embodiments, R⁷ is C(O)C1-C6 alkyl. For example, R⁷ is C(O)Me. In some embodiments, R⁷ is CO₂C1-C6 alkyl. For example, R⁷ is CO₂Me. In some embodiments, R⁷ is C(O)NHC1-C6 alkyl. For example, R⁷ is C(O)NHMe. In some embodiments, R⁷ is N(R′)₂. For example, R⁷ is NH₂, NHMe, or NMe₂.

In some embodiments, R⁸ is halogen. In some embodiments, R⁸ is fluoro or chloro. In some embodiments, R⁸ is pseudohalogen.

In some embodiments, R⁸ is C1-C6 alkoxy. For example, R⁸ is methoxy. In some embodiments, R⁸ is cyano. In some embodiments, R⁸ is nitro. In some embodiments, R⁸ is C(O)C1-C6 alkyl. For example, R⁸ is C(O)Me. In some embodiments, R⁸ is CO₂C1-C6 alkyl. For example, R⁸ is CO₂Me. In some embodiments, R⁸ is C(O)NHC1-C6 alkyl. For example, R⁸ is C(O)NHMe. In some embodiments, R⁸ is N(R′)₂. For example, R⁸ is NH₂, NHMe, or NMe₂.

In some embodiments, R⁸ is C1-C10 alkyl optionally substituted with 1-6 halogens. In some embodiments, R⁸ is C1-C6 alkyl optionally substituted with 1-6 halogens. In some embodiments, R⁸ is C1-C6 alkyl optionally substituted with 1-3 halogens. In some embodiments, R⁸ is C1-C3 alkyl optionally substituted with 1-3 halogens. In some embodiments, R⁸ is C1-C6 alkyl substituted with 1-6 halogens. In some embodiments, R⁸ is C1-C6 alkyl substituted with 1-3 halogens. In some embodiments, R⁸ is C1-C3 alkyl substituted with 1-3 halogens. In some embodiments, R⁸ is an unsubstituted C1-C10 alkyl. In some embodiments, R⁸ is an unsubstituted C1-C6 alkyl. In some embodiments, R⁸ is an unsubstituted C1-C3 alkyl. In some embodiments, R⁸ is methyl. In some embodiments, R⁸ is trifluoromethyl.

In some embodiments, R⁸ is C6-C10 aryl. In some embodiments, R⁸ is 5 to 10 membered heteroaryl.

In some embodiments, R⁸ is C1-C6 alkoxy. For example, R⁸ is methoxy. In some embodiments, R⁸ is cyano. In some embodiments, R⁸ is nitro. In some embodiments, R⁸ is C(O)C1-C6 alkyl. For example, R⁸ is C(O)Me. In some embodiments, R⁸ is CO₂C1-C6 alkyl. For example, R⁸ is CO₂Me. In some embodiments, R⁸ is C(O)NHC1-C6 alkyl. For example, R⁸ is C(O)NHMe. In some embodiments, R⁸ is N(R′)₂. For example, R⁸ is NH₂, NHMe, or NMe₂.

In some embodiments, R⁹ is halogen. In some embodiments, R⁹ is fluoro or chloro.

In some embodiments, R⁹ is pseudohalogen. In some embodiments, R⁹ is C1-C10 alkyl optionally substituted with 1-6 halogens. In some embodiments, R⁹ is methyl. In some embodiments, R⁹ is trifluoromethyl. In some embodiments, R⁹ is C6-C10 aryl. In some embodiments, R⁹ is 5 to 10 membered heteroaryl.

In some embodiments, R⁹ is C1-C6 alkoxy. For example, R⁹ is methoxy. In some embodiments, R⁹ is cyano. In some embodiments, R⁹ is nitro. In some embodiments, R⁹ is C(O)C1-C6 alkyl. For example, R⁹ is C(O)Me. In some embodiments, R⁹ is CO₂C1-C6 alkyl. For example, R⁹ is CO₂Me. In some embodiments, R⁹ is C(O)NHC1-C6 alkyl. For example, R⁹ is C(O)NHMe. In some embodiments, R⁹ is N(R′)₂. For example, R⁹ is NH₂, NHMe, or NMe₂.

In some embodiments, R¹⁰ is halogen. In some embodiments, R¹⁰ is fluoro or chloro. In some embodiments, R¹⁰ is pseudohalogen.

In some embodiments, R¹⁰ is C1-C10 alkyl optionally substituted with 1-6 halogens. In some embodiments, R¹⁰ is C1-C6 alkyl optionally substituted with 1-6 halogens. In some embodiments, R¹⁰ is C1-C6 alkyl optionally substituted with 1-3 halogens. In some embodiments, R¹⁰ is C1-C3 alkyl optionally substituted with 1-3 halogens. In some embodiments, R¹⁰ is C1-C6 alkyl substituted with 1-6 halogens. In some embodiments, R¹⁰ is C1-C6 alkyl substituted with 1-3 halogens. In some embodiments, R¹⁰ is C1-C3 alkyl substituted with 1-3 halogens. In some embodiments, R¹⁰ is an unsubstituted C1-C10 alkyl. In some embodiments, R¹⁰ is an unsubstituted C1-C6 alkyl. In some embodiments, R¹⁰ is an unsubstituted C1-C3 alkyl. In some embodiments, R¹⁰ is methyl. In some embodiments, R¹⁰ is trifluoromethyl.

In some embodiments, R¹⁰ is C6-C10 aryl. In some embodiments, R¹⁰ is 5 to 10 membered heteroaryl.

In some embodiments, R¹⁰ is C1-C6 alkoxy. For example, R¹⁰ is methoxy. In some embodiments, R¹⁰ is cyano. In some embodiments, R¹⁰ is nitro. In some embodiments, R¹⁰ is C(O)C1-C6 alkyl. For example, R¹⁰ is C(O)Me. In some embodiments, R¹⁰ is CO₂C1-C6 alkyl. For example, R¹⁰ is CO₂Me. In some embodiments, R¹⁰ is C(O)NHC1-C6 alkyl. For example, R¹⁰ is C(O)NHMe. In some embodiments, R¹⁰ is N(R′)₂. For example, R¹⁰ is NH₂, NHMe, or NMe₂.

In some embodiments, each R′ is H. In some embodiments, one R′ is H and the other R′ is C1-C6 alkyl. For example, one R′ is H and the other R′ is methyl. In some embodiments, each R′ is C1-C6 alkyl. For example, each R′ is methyl.

In some embodiments, one of R⁵, R⁶, R⁷, R⁸, R⁹, and R¹⁰ is hydrogen. In some embodiments, two of R⁵, R⁶, R⁷, R⁸, R⁹, and R¹⁰ are hydrogen. In some embodiments, three of R⁵, R⁶, R⁷, R⁸, R⁹, and R¹⁰ are hydrogen. In some embodiments, four of R⁵, R⁶, R¹, R⁷, R⁹, and R¹⁰ are hydrogen. In some embodiments, four of R⁵, R⁶, R⁷, R⁸, R⁹, and R¹⁰ are hydrogen; and the remaining two of R⁵, R⁶, R⁷, R⁸, R⁹, and R¹⁰ are independently selected from halogen and unsubstituted C1-C6 alkyl.

In some embodiments, when there are greater than one R², the greater than one R² are the same. In some embodiments, R⁵ and R¹⁰ are the same. In some embodiments, R⁵ and R¹⁰ are different. In some embodiments, R⁵ and R¹⁰ are independently selected from hydrogen, methyl, and fluoro. In some embodiments, R⁵ and R¹⁰ are the same, and are selected from hydrogen, methyl, and fluoro. In some embodiments, R⁵ and R¹⁰ are both hydrogen. In some embodiments, R⁶ and one of R⁸ and R⁹ are the same, and R⁷ and the other of R⁸ and R⁹ are the same.

In some embodiments, n is 2, and each R² is hydrogen. In some embodiments, n is 2, and each R² is hydroxyl. In some embodiments, R⁶, R⁷, R⁸, and R⁹ are independently selected from methyl, trifluoromethyl, fluoro, and chloro. In some embodiments, n is 2, each R² is hydrogen, and R⁵ and R¹⁰ are both hydrogen.

Some embodiments provide compounds of Formula (II-a), or pharmaceutically acceptable salts thereof,

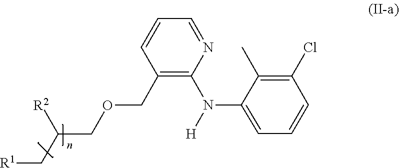

wherein:
   R¹ is selected from OH and

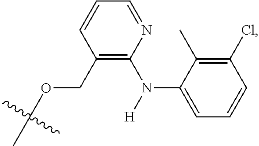

wherein [symbol] represents the point of connection of R¹ to the remainder of the molecule;
n is 0, 1, 2, or 3;
each occurrence of R² is an independently selected hydrogen or OH.

In some embodiments, the compound is:

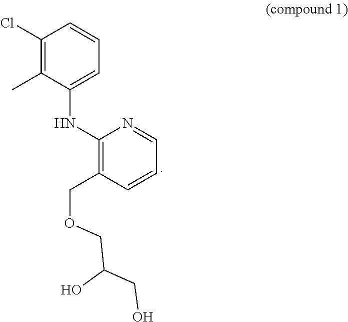 [SMILES: Cc1c(Cl)cccc1Nc1ncccc1COCC(O)CO]

In some embodiments, the compound is:

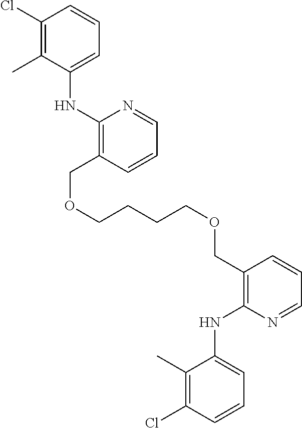 [SMILES: Cc1c(Cl)cccc1Nc1ncccc1COCCCCOCc1cccnc1Nc1cccc(Cl)c1C]

(compound 2).

In some embodiments, the compound is:

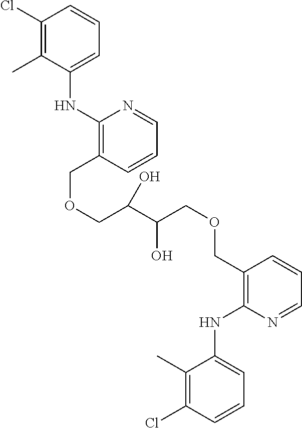 [SMILES: Cc1c(Cl)cccc1Nc1ncccc1COCC(O)C(O)COCc1cccnc1Nc1cccc(Cl)c1C]

(compound 3).

In some embodiments, compounds of Formula (II), or pharmaceutically acceptable salts thereof, function as STING agonists. In other embodiments, compounds of Formula (II), or pharmaceutically acceptable salts thereof, function as partial STING agonists. In still other embodiments, compounds of Formula (II), or pharmaceutically acceptable salts thereof, partial or full STING antagonists.

Some embodiments provide compounds of Formula (III),

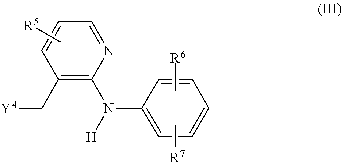

wherein:
   Y^{A} is selected from OH and NR¹¹R¹²
   R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl;
   each occurrence of R′ is independently selected from H and C1-C6 alkyl; and
   R¹¹ and R¹² are independently selected from H, C1-C3 alkyl, and C(O)C1-C3 alkyl.

Some embodiments provide compounds of Formula (III),

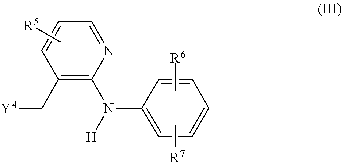

wherein:
   Y^{A} is selected from OH and NR¹¹R¹²;
   R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl;
   each occurrence of R′ is independently selected from H and C1-C6 alkyl; R¹¹ and R¹² are independently selected from H, C1-C3 alkyl, and C(O)C1-C3 alkyl; and
   wherein the compound is not

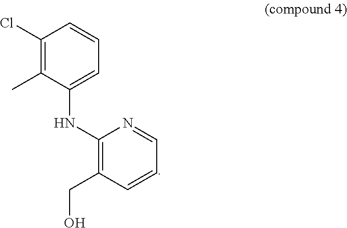 [SMILES: Cc1c(Cl)cccc1Nc1ncccc1CO]

In some embodiments, Y^{A} is OH. In some embodiments, Y^{A} is NR¹¹R¹².

Some embodiments, provide compounds of Formula (III-a),

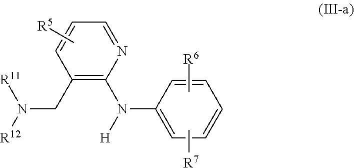

wherein:
   R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl;
   each occurrence of R′ is independently selected from H and C1-C6 alkyl; and
   R¹¹ and R¹² are independently selected from H, C1-C3 alkyl, and C(O)C1-C3 alkyl.

In some embodiments, each R¹¹ and R¹² is H. In some embodiments, one of R¹¹ and R¹² is H and the other of R¹¹ and R¹² is C1-C3 alkyl. In some embodiments, one of R¹¹ and R¹² is H and the other of R¹¹ and R¹² is methyl. In some embodiments, each R¹¹ and R¹² is C1-C3 alkyl. In some embodiments, each R¹¹ and R¹² is methyl. In some embodiments, one of R¹¹ and R¹² is H and the other of R¹¹ and R¹² is C(O)C1-C3 alkyl. In some embodiments, one of R¹¹ and R¹² is H and the other of R¹¹ and R¹² is C(O)CH₃.

In some embodiments, R⁵, R⁶, R⁷, R⁸, R⁹, and R¹⁰ are as defined anywhere else herein.

In some embodiments, the compound is selected from the group consisting of:

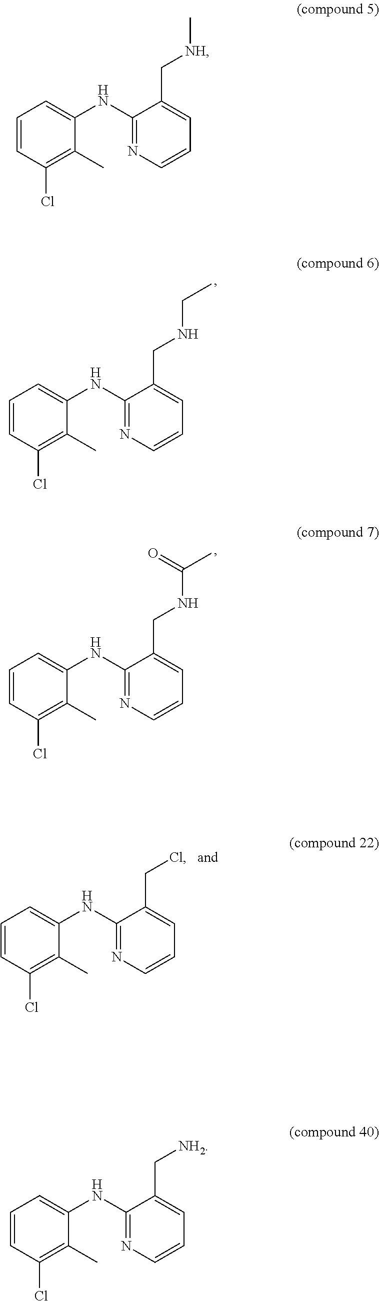 [SMILES: CNCc1cccnc1Nc1cccc(Cl)c1C ; CCNCc1cccnc1Nc1cccc(Cl)c1C ; CC(=O)NCc1cccnc1Nc1cccc(Cl)c1C ; Cc1c(Cl)cccc1Nc1ncccc1CCl ; "and" ; Cc1c(Cl)cccc1Nc1ncccc1CN]

In some embodiments, the compound is not

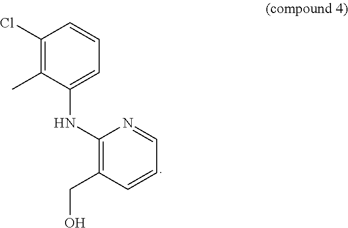 [SMILES: Cc1c(Cl)cccc1Nc1ncccc1CO]

In some embodiments, compounds of Formula (III), or pharmaceutically acceptable salts thereof, function as STING agonists. In other embodiments, compounds of Formula (III), or pharmaceutically acceptable salts thereof, function as partial STING agonists. In still other embodiments, compounds of Formula (III), or pharmaceutically acceptable salts thereof, partial or full STING antagonists.

### Methods of Use

Some embodiments provide a method of agonizing or partially agonizing the STING pathway in a subject in need thereof, comprising administering a compound of Formula (I), Formula (II), and/or Formula (III) (e.g., Formula (I)), or a pharmaceutically acceptable salt thereof to the subject. In some embodiments, agonizing or partially agonizing the STING pathway in a subject comprises increasing production of IFN-β in the subject. In some embodiments, agonizing or partially agonizing the STING pathway in a subject comprises activating or increasing T-cell immunity. In some embodiments, agonizing or partially agonizing the STING pathway in a subject comprises activating or increasing CD8⁺ T-cell immunity. In some embodiments, agonizing or partially agonizing the STING pathway in a subject comprises activating or increasing tumor antigen-specific CD8⁺ T-cell immunity. In some embodiments, increasing T-cell immunity comprises increasing the T-cell population. In some embodiments, the T-cell population is increased by about 20% to about 200%, for example, about 20% to about 80%, about 60% to about 120%, about 100% to about 160%, about 140% to about 200%, or any value in between.

Some embodiments provide a method of antagonizing the STING pathway in a subject in need thereof, comprising administering a compound of Formula (I), Formula (II), and/or Formula (III) (e.g., Formula (I)), or a pharmaceutically acceptable salt thereof, to the subject. In some embodiments, antagonizing the STING pathway in a subject comprises reducing expression of IFN-β in the subject. In some embodiments, antagonizing the STING pathway in a subject comprises reducing expression of one or more cytokines in the subject. In some embodiments, production of IFN-β is decreased by about 10% to about 99%, for example, about 10% to about 30%, about 20% to about 40%, about 30% to about 50%, about 40% to about 60%, about 50% to about 70%, about 60% to about 80%, about 70% to about 90%, about 80% to about 99%, or any value in between. In some embodiments, antagonizing the STING pathway in a subject comprises reducing expression of one or more cytokines in the subject. In some embodiments, the expression of one or more cytokines is decreased by about 10% to about 99%, for example, about 10% to about 30%, about 20% to about 40%, about 30% to about 50%, about 40% to about 60%, about 50% to about 70%, about 60% to about 80%, about 70% to about 90%, about 80% to about 99%, or any value in between. In some embodiments, the one or more cytokines (e.g., two or more, five or more, ten or more, up to twenty or fifty) cytokines. In some embodiments, the one or more cytokines are induced by IRF3. In some embodiments, the one or more cytokines are induced by NF-κB. In some embodiments, antagonizing the STING pathway in a subject comprises reducing expression of one or more of TOR1B, C1orf29, FAM3B, OAS3, USP18, OAS1, Siglec-1, GBP5, IFIT5, IFIT2, IFRG28, IFIT1, PRKR (EIF2AK1), IL1RN, OASL, OAS1, LGALS3BP, OASL, IFIH1 (MDA5), ZBP1, C1QB, CEB1, GBP1, BST2, IFI44, IFI27, GBP2, EPSTIl, CARD15, IFI35, SOCS1, TAP1, XAF1, SP110, OAS2, STAT1, ABCA1, IFIT4, PLSCR1, Cig5, ISG95, STAT2, RIG-I (DDX58), MX2, LGP2, IRF7, ADD45B, SCOTIN, PARP9 (BAL), MT2A, NT5C3 (PN-1), MX1, STAT1, ADAR, TRIM22, G1P2, SERPING1, STAT1, NUB1 (NYREN18), ISG20, LY6E, G1P3, and/or IFITM1 in the subject. In some embodiments, antagonizing the STING pathway in a subject comprises reducing expression of one or more of CCL5, HIF1z, CXCL10, C1orf29, GMPR, IL-6, OAS, IFITM1, TOR1B, OAS1, IFI138, FAM3B, GBP5, IF144L, USP18, IFIT2, PLSCR1, Siglec-1, EPSTIl, TMEM255A, IFIT5, IFI35, SNRPE, IFRG28, TAP1, SNRPA1, PRKR (EIF2AK1), OASL, SNRPB, OASL, ZBP1, SNRPB2, LGALS3BP, CEBlSNRPC, MDA5, BST2, SNRPE, C1QB, IFI2, SNRPF, LGALS3BP, SP110, IRF1, GBP1, STAT1, IRF2, IFI44, IFIT4, Tnfa, GBP2, Cig5, GM-CSF, CARD15, STAT2, IL-13, SOSCS1, MX2, IL-10, XAF1, IRF7, ddx41, OAS2, COTIN, DAI, ABCA1, MT2A, MRE11, PLSCR1, MX1, ISG95, ADAR, RIG-I (DDX58), GIP2, LGP2, LY6E, GADD45B, LAMP3, PARP9 (BAL), CCL2, NT5C3, IF127, STAT1, HSPA1A, TRIM22, HSPA1B, SERPING1, HAPA2, and/or NUB1.

In some embodiments, antagonizing the STING pathway in a subject comprises reducing expression of one or more of STAT1, STAT2, Cig5, G1P3, IRF7, IFIT4, Ly6E, MX1, OAS3 and/or IFI27 in the subject. In some embodiments, the one or more cytokines are induced by IRF3 and NF-κB. In some embodiments, antagonizing the STING pathway in a subject comprises reducing anti-DNA antibody production.

Some embodiments provide a method of treating a viral infection, comprising administering a therapeutically effective amount of a compound of Formula (I), Formula (II), and/or Formula (III) (e.g., Formula (I)), or a pharmaceutically acceptable salt thereof, to a subject in need thereof. Some embodiments provide a method of treating a viral infection comprising administering a therapeutically effective amount of a compound of Formula (II), or a pharmaceutically acceptable salt thereof. In some embodiments, the virus is a DNA virus. In some embodiments, the virus is an RNA virus. In some embodiments, the virus is a coronavirus, such as SARS-Cov2.

Some embodiments provide a method of treating cancer, comprising administering a therapeutically effective amount of a compound of Formula (I), Formula (II), and/or Formula (III) (e.g., Formula (I)), or a pharmaceutically acceptable salt thereof, to a subject in need thereof. Some embodiments provide a method of treating cancer comprising administering a therapeutically effective amount of a compound of Formula (I), or a pharmaceutically acceptable salt thereof. Some embodiments provide a method of treating cancer comprising administering a therapeutically effective amount of a compound of Formula (II), or a pharmaceutically acceptable salt thereof.

In some embodiments the cancer is a solid tumor. Non-limiting examples of solid tumors include, for example, thyroid cancer (e.g., papillary thyroid carcinoma, medullary thyroid carcinoma), lung cancer (e.g., lung adenocarcinoma, small-cell lung carcinoma), pancreatic cancer, pancreatic ductal carcinoma, breast cancer, colon cancer, colorectal cancer, prostate cancer, renal cell carcinoma, head and neck tumors, neuroblastoma, and melanoma. See, for example, Nature Reviews Cancer, 2014, 14, 173-186, which is incorporated by reference herein in its entirety.

In other embodiments, the cancer is a blood cancer. Non-limiting examples of blood cancers include leukemia, myeloma, and lymphoma.

In some embodiments, the cancer is selected from the group consisting of lung cancer, papillary thyroid cancer, medullary thyroid cancer, differentiated thyroid cancer, recurrent thyroid cancer, refractory differentiated thyroid cancer, multiple endocrine neoplasia type 2A or 2B (MEN2A or MEN2B, respectively), pheochromocytoma, parathyroid hyperplasia, breast cancer, colorectal cancer, papillary renal cell carcinoma, ganglioneuromatosis of the gastroenteric mucosa, and cervical cancer.

In some embodiments, the subject is a human.

In some embodiments, treating the cancer comprises agonizing or partially agonizing STING. In some embodiments, treating the cancer comprises increasing production of IFN-β in the subject. In some embodiments, treating the cancer comprises activating or increasing T-cell immunity. In some embodiments, treating the cancer comprises activating or increasing CD8⁺ T-cell immunity. In some embodiments, treating the cancer comprises activating or increasing tumor antigen-specific CD8⁺ T-cell immunity. In some embodiments, increasing T-cell immunity comprises increasing T-cell population.

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other kinase inhibitors, signal transduction inhibitors and/or monoclonal antibodies. For example, a surgery may be open surgery or minimally invasive surgery. Compounds of Formula (I), Formula (II), and/or Formula (III) (e.g., Formula (I)), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), Formula (II), and/or Formula (III) (e.g., Formula (I)), or a pharmaceutically acceptable salt thereof, can be used prior to administration of an additional therapeutic agent or additional therapy. For example, a subject in need thereof can be administered one or more doses of a compound of Formula (I), Formula (II), and/or Formula (III) (e.g., Formula (I)), or a pharmaceutically acceptable salt thereof for a period of time and then undergo at least partial resection of the tumor. In some embodiments, the treatment with one or more doses of a compound of Formula (I), Formula (II), and/or Formula (III) (e.g., Formula (I)), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), Formula (II), and/or Formula (III) (e.g., Formula (I)), or a pharmaceutically acceptable salt thereof for a period of time and under one or more rounds of radiation therapy. In some embodiments, the treatment with one or more doses of a compound of Formula (I), Formula (II), and/or Formula (III) (e.g., Formula (I)), or a pharmaceutically acceptable salt thereof reduces the size of the tumor (e.g., the tumor burden) prior to the one or more rounds of radiation therapy.

In some embodiments of any of the methods described herein, the compound of Formula (I), Formula (II), and/or Formula (III) (e.g., Formula (I)), or a pharmaceutically acceptable salt thereof, is administered in combination with an effective amount of at least one additional therapeutic agent selected from one or more additional therapies or therapeutic (e.g., chemotherapeutic) agents.

Non-limiting examples of additional therapeutic agents include: other STING agonists or partial agonists, kinase inhibitors (e.g., receptor tyrosine kinase-targeted therapeutic agents (e.g., Trk inhibitors or EGFR inhibitors) or multi-kinase inhibitors), signal transduction pathway inhibitors, checkpoint inhibitors, modulators of the apoptosis pathway (e.g., obataclax); cytotoxic chemotherapeutics, angiogenesis-targeted therapies, immune-targeted agents, including immunotherapy, and radiotherapy.

Non-limiting examples of multi-kinase inhibitors include alectinib (9-Ethyl-6,6-dimethyl-8-[4-(morpholin-4-yl)piperidin-1-yl]-11-oxo-6,11-dihydro-5H-benzo[b]carbazole-3-carbonitrile); amuvatinib (MP470, HPK56) (N-(1,3-benzodioxol-5-ylmethyl)-4-([1]benzofuro[3,2-d]pyrimidin-4-yl)piperazine-1-carbothioamide); apatinib (YN968D1) (N-[4-(1-cyanocyclopentyl) phenyl-2-(4-picolyl)amino-3-Nicotinamide methanesulphonate); cabozantinib (Cometriq XL-184) (N-(4-((6,7-Dimethoxyquinolin-4-yl)oxy)phenyl)-N′-(4-fluorophenyl)cyclopropane-1,1-dicarboxamide); dovitinib (TKI258; GFKI-258; CHIR-258) ((3Z)-4-amino-5-fluoro-3-[5-(4-methylpiperazin-1-yl)-1,3-dihydrobenzimidazol-2-ylidene]quinolin-2-one); famitinib (5-[2-(diethylamino)ethyl]-2-[(Z)-(5-fluoro-2-oxo-1H-indol-3-ylidene)methyl]-3-methyl-6,7-dihydro-1H-pyrrolo[3,2-c]pyridin-4-one); fedratinib (SAR302503, TG101348) (N-(2-Methyl-2-propanyl)-3-{[5-methyl-2-({4-[2-(1-pyrrolidinyl)ethoxy]phenyl}amino)-4-pyrimidinyl]amino}benzenesulfonamide); foretinib (XL880, EXEL-2880, GSK1363089, GSK089) (N1′-[3-fluoro-4-[[6-methoxy-7-(3-morpholinopropoxy)-4-quinolyl]oxy]phenyl]-N1-(4-fluorophenyl)cyclopropane-1,1-dicarboxamide); fostamantinib (R788) (2H-Pyrido[3,2-b]-1,4-oxazin-3(4H)-one, 6-[[5-fluoro-2-[(3,4,5-trimethoxyphenyl)amino]-4-pyrimidinyl]amino]-2,2-dimethyl-4-[(phosphonooxy)methyl]-, sodium salt (1:2)); ilorasertib (ABT-348) (1-(4-(4-amino-7-(1-(2-hydroxyethyl)-1H-pyrazol-4-yl)thieno[3,2-c]pyridin-3-yl)phenyl)-3-(3-fluorophenyl)urea); lenvatinib (E7080, Lenvima) (4-[3-chloro-4-(cyclopropylaminocarbonyl)aminophenoxy]-7-methoxy-6-quinolinecarboxamide); motesanib (AMG 706) (N-(3,3-Dimethyl-2,3-dihydro-1H-indol-6-yl)-2-[(pyridin-4-ylmethyl)amino]pyridine-3-carboxamide); nintedanib (3-Z-[1-(4-(N-((4-methyl-piperazin-1-yl)-methylcarbonyl)-N-methyl-amino)-anilino)-1-phenyl-methylene]-6-methyoxycarbonyl-2-indolinone); ponatinib (AP24534) (3-(2-Imidazo[1,2-b]pyridazin-3-ylethynyl)-4-methyl-N-[4-[(4-methylpiperazin-1-yl)methyl]-3-(trifluoromethyl)phenyl]benzamide); PP242 (torkinib) (2-[4-Amino-1-(1-methylethyl)-1H-pyrazolo[3,4-d]pyrimidin-3-yl]-1H-indol-5-ol); quizartinib (1-(5-(tert-Butyl)isoxazol-3-yl)-3-(4-(7-(2-morpholinoethoxy)benzo[d]imidazo[2,1-b]thiazol-2-yl)phenyl)urea); regorafenib (BAY 73-4506, stivarga) (4-[4-({[4-Chloro-3-(trifluoromethyl)phenyl]carbamoyl}amino)-3-fluorophenoxy]-N-methylpyridine-2-carboxamide hydrate); RXDX-105 (CEP-32496, agerafenib) (1-(3-((6,7-dimethoxyquinazolin-4-yl)oxy)phenyl)-3-(5-(1,1,1-trifluoro-2-methylpropan-2-yl)isoxazol-3-yl)urea); semaxanib (SU5416) ((3Z)-3-[(3,5-dimethyl-1H-pyrrol-2-yl)methylidene]-1,3-dihydro-2H-indol-2-one); sitravatinib (MGCD516, MG516) (N-(3-Fluoro-4-{[2-(5-{[(2-methoxyethyl)amino]methyl}-2-pyridinyl)thieno[3,2-b]pyridin-7-yl]oxy}phenyl)-N′-(4-fluorophenyl)-1,1-cyclopropanedicarboxamide); sorafenib (BAY 43-9006) (4-[4-[[[[4-chloro-3-(trifluoromethyl)phenyl]amino]carbonyl]amino]phenoxy]-N-methyl-2-pyridinecarboxamide); vandetanib (N-(4-bromo-2-fluorophenyl)-6-methoxy-7-[(1-methylpiperidin-4-yl)methoxy]quinazolin-4-amine); vatalanib (PTK787, PTK/ZK, ZK222584) (N-(4-chlorophenyl)-4-(pyridin-4-ylmethyl)phthalazin-1-amine); AD-57 (N-[4-[4-amino-1-(1-methylethyl)-1H-pyrazolo[3,4-d]pyrimidin-3-yl]phenyl]-N′-[3-(trifluoromethyl)phenyl]-urea); AD-80 (1-[4-(4-amino-1-propan-2-ylpyrazolo[3,4-d]pyrimidin-3-yl)phenyl]-3-[2-fluoro-5-(trifluoromethyl)phenyl]urea); AD-81 (1-(4-(4-amino-1-isopropyl-1H-pyrazolo[3,4-d]pyrimidin-3-yl)phenyl)-3-(4-chloro-3-(trifluoromethyl)phenyl)urea); ALW-II-41-27 (N-(5-((4-((4-ethylpiperazin-1-yl)methyl)-3-(trifluoromethyl)phenyl)carbamoyl)-2-methylphenyl)-5-(thiophen-2-yl)nicotinamide); BPR1K871 (1-(3-chlorophenyl)-3-(5-(2-((7-(3-(dimethylamino)propoxy)quinazolin-4-yl)amino)ethyl)thiazol-2-yl)urea); CLM3 (1-phenethyl-N-(1-phenylethyl)-1H-pyrazolo[3,4-d]pyrimidin-4-amine); EBI-907 (N-(2-chloro-3-(1-cyclopropyl-8-methoxy-3H-pyrazolo[3,4-c]isoquinolin-7-yl)-4-fluorophenyl)-3-fluoropropane-1-sulfonamide); NVP-AST-487 (N-[4-[(4-ethyl-1-piperazinyl)methyl]-3-(trifluoromethyl)phenyl]-N′-[4-[[6-(methylamino)-4-pyrimidinyl]oxy]phenyl]-urea); NVP-BBT594 (BBT594) (5-((6-acetamidopyrimidin-4-yl)oxy)-N-(4-((4-methylpiperazin-1-yl)methyl)-3-(trifluoromethyl)phenyl)indoline-1-carboxamide); PD173955 (6-(2,6-dichlorophenyl)-8-methyl-2-(3-methylsulfanylanilino)pyrido[2,3-d]pyrimidin-7-one); PP2 (4-amino-5-(4-chlorophenyl)-7-(dimethylethyl)pyrazolo[3,4-d]pyrimidine); PZ-1 (N-(5-(tert-butyl)isoxazol-3-yl)-2-(4-(5-(1-methyl-1H-pyrazol-4-yl)-1Hbenzo[d]imidazol-1-yl)phenyl)acetamide); RPI-1 (1,3-dihydro-5,6-dimethoxy-3-[(4-hydroxyphenyl)methylene]-H-indol-2-one; (3E)-3-[(4-hydroxyphenyl)methylidene]-5,6-dimethoxy-1H-indol-2-one); SGI-7079 (3-[2-[[3-fluoro-4-(4-methyl-1-piperazinyl)phenyl]amino]-5-methyl-7H-pyrrolo[2,3-d]pyrimidin-4-yl]-benzeneacetonitrile); SPP86 (1-Isopropyl-3-(phenylethynyl)-1H-pyrazolo[3,4-d]pyrimidin-4-amine); SU4984 (4-[4-[(E)-(2-oxo-1H-indol-3-ylidene)methyl]phenyl]piperazine-1-carbaldehyde); sunitinb (SU11248) (N-(2-Diethylaminoethyl)-5-[(Z)-(5-fluoro-2-oxo-1H-indol-3-ylidene)methyl]-2,4-dimethyl-1H-pyrrole-3-carboxamide); TG101209 (N-tert-butyl-3-(5-methyl-2-(4-(4-methylpiperazin-1-yl)phenylamino)pyrimidin-4-ylamino)benzenesulfonamide); Withaferin A ((40,50,60,22R)-4,27-Dihydroxy-5,6:22,26-diepoxyergosta-2,24-diene-1,26-dione); XL-999 ((Z)-5-((1-ethylpiperidin-4-yl)amino)-3-((3-fluorophenyl)(5-methyl-1H-imidazol-2-yl)methylene)indolin-2-one); BPR1J373 (a 5-phenylthiazol-2-ylamine-pyriminide derivative); CG-806 (CG'806); DCC-2157; GTX-186; HG-6-63-01 ((E)-3-(2-(4-chloro-1H-pyrrolo[2,3-b]pyridin-5-yl)vinyl)-N-(4-((4-ethylpiperazin-1-yl)methyl)-3-(trifluoromethyl)phenyl)-4-methylbenzamide); SW-01 (Cyclobenzaprine hydrochloride); XMD15-44 (N-(4-((4-ethylpiperazin-1-yl)methyl)-3-(trifluoromethyl)phenyl)-4-methyl-3-(pyridin-3-ylethynyl)benzamide (generated from structure)); Y078-DM1 (an antibody drug conjugate composed of a CDC7 antibody (Y078) linked to a derivative of the cytotoxic agent maytansine); Y078-DM4 (an antibody drug conjugate composed of a CDC7 antibody (Y078) linked to a derivative of the cytotoxic agent maytansine); ITRI-305 (D0N5 TB, DIB003599); BLU-667 ((1S,4R)-N-((S)-1-(6-(4-fluoro-1H-pyrazol-1-yl)pyridin-3-yl)ethyl)-1-methoxy-4-(4-methyl-6-((5-methyl-1H-pyrazol-3-yl)amino)pyrimidin-2-yl)cyclohexane-1-carboxamide); BLU6864; DS-5010; GSK3179106; GSK3352589; NMS-E668; TAS0286/HM05; TPX0046; and N-(3-(2-(dimethylamino)ethoxy)-5-(trifluoromethyl)phenyl)-2-(4-(4-ethoxy-6-oxo-1,6-dihydropyridin-3-yl)-2-fluorophenyl)acetamide.

Non-limiting examples of receptor tyrosine kinase (e.g., Trk) targeted therapeutic agents, include afatinib, cabozantinib, cetuximab, crizotinib, dabrafenib, entrectinib, erlotinib, gefitinib, imatinib, lapatinib, lestaurtinib, nilotinib, pazopanib, panitumumab, pertuzumab, sunitinib, trastuzumab, 1-((3S,4R)-4-(3-fluorophenyl)-1-(2-methoxyethyl)pyrrolidin-3-yl)-3-(4-methyl-3-(2-methylpyrimidin-5-yl)-1-phenyl-1H-pyrazol-5-yl)urea, AG 879, AR-772, AR-786, AR-256, AR-618, AZ-23, AZ623, DS-6051, Go 6976, GNF-5837, GTx-186, GW 441756, LOXO-101, MGCD516, PLX7486, RXDX101, VM-902A, TPX-0005, TSR-011, GNF-4256, N-[3-[[2,3-dihydro-2-oxo-3-(1H-pyrrol-2-ylmethylene)-1H-indol-6-yl]amino]-4-methylphenyl]-N′-[2-fluoro-5-(trifluoromethyl)phenyl]-urea, AZ623, AZ64, (S)-5-Chloro-N2-(1-(5-fluoropyridin-2-yl)ethyl)-N4-(5-isopropoxy-1H-pyrazol-3-yl)pyrimidine-2,4-diamine, AZD7451, CEP-751, CT327, sunitinib, GNF-8625, and (R)-1-(6-(6-(2-(3-fluorophenyl)pyrrolidin-1-yl)imidazo[1,2-b]pyridazin-3-yl)-[2,4′-bipyridin]-2′-yl)piperidin-4-ol.

Non-limiting examples of a BRAF inhibitor include dabrafenib, vemurafenib (also called RG7204 or PLX4032), sorafenib tosylate, PLX-4720, GDC-0879, BMS-908662 (Bristol-Meyers Squibb), LGX818 (Novartis), PLX3603 (Hofmann-LaRoche), RAF265 (Novartis), RO5185426 (Hofmann-LaRoche), and GSK2118436 (GlaxoSmithKline). Additional examples of a BRAF inhibitor are known in the art.

In some embodiments, the receptor tyrosine kinase inhibitor is an epidermal growth factor receptor typrosine kinase inhibitor (EGFR). For example, EGFR inhibitors can include osimertinib (merelectinib, Tagrisso), erlotinib (Tarceva), gefitinib (Iressa), cetuximab (Erbitux), necitumumab (Portrazza), neratinib (Nerlynx), lapatinib (Tykerb), panitumumab (Vectibix), and vandetanib (Caprelsa).

In some embodiments, signal transduction pathway inhibitors include Ras-Raf-MEK-ERK pathway inhibitors (e.g., binimetinib, selumetinib, encorafenib, sorafenib, trametinib, and vemurafenib), PI3K-Akt-mTOR-S6K pathway inhibitors (e.g., everolimus, rapamycin, perifosine, temsirolimus), and other kinase inhibitors, such as baricitinib, brigatinib, capmatinib, danusertib, ibrutinib, milciclib, quercetin, regorafenib, ruxolitinib, semaxanib, AP32788, BLU285, BLU554, INCB39110, INCB40093, INCB50465, INCB52793, INCB54828, MGCD265, NMS-088, NMS-1286937, PF 477736 ((R)-amino-N-[5,6-dihydro-2-(1-methyl-1H-pyrazol-4-yl)-6-oxo-1Hpyrrolo[4,3,2-ef][2,3]benzodiazepin-8-yl]-cyclohexaneacetamide), PLX3397, PLX7486, PLX8394, PLX9486, PRN1008, PRN1371, RXDX103, RXDX106, RXDX108, and TG101209 (N-tert-butyl-3-(5-methyl-2-(4-(4-methylpiperazin-1-yl)phenylamino)pyrimidin-4-ylamino)benzenesulfonamide).

Non-limiting examples of checkpoint inhibitors include ipilimumab, tremelimumab, nivolumab, pidilizumab, MPDL3208A, MEDI4736, MSB0010718C, BMS-936559, BMS-956559, BMS-935559 (MDX-1105), AMP-224, and pembrolizumab.

In some embodiments, cytotoxic chemotherapeutics are selected from arsenic trioxide, bleomycin, cabazitaxel, capecitabine, carboplatin, cisplatin, cyclophosphamide, cytarabine, dacarbazine, daunorubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

Non-limiting examples of angiogenesis-targeted therapies include aflibercept and bevacizumab.

In some embodiments, an additional therapy or therapeutic agent can include a histidyl-tRNA synthetase (HRS) polypeptide or an expressible nucleotide that encodes the HRS polypeptide.

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymriah™).

In some embodiments, the immunotherapy is an antibody therapy (e.g., a monoclonal antibody, a conjugated antibody). In some embodiments, the antibody therapy is bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™), cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab or amatuximab.

In some embodiments, the immunotherapy is an antibody-drug conjugate. In some embodiments, the antibody-drug conjugate is gemtuzumab ozogamicin (Mylotarg™) inotuzumab ozogamicin (Besponsa®), brentuximab vedotin (Adcetris®), ado-trastuzumab emtansine (TDM-1; Kadcyla®), mirvetuximab soravtansine (IMGN853) or anetumab ravtansine

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is IntronA® (Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

In some embodiments, the immunotherapy is mRNA-based immunotherapy. In some embodiments, the mRNA-based immunotherapy is CV9104 (see, e.g., Rausch et al. (2014) Human Vaccin Immunother 10(11): 3146-52; and Kubler et al. (2015) J. Immunother Cancer 3:26).

In some embodiments, the immunotherapy is bacillus Calmette-Guerin (BCG) therapy.

In some embodiments, the immunotherapy is an oncolytic virus therapy. In some embodiments, the oncolytic virus therapy is talimogene alherparepvec (T-VEC; Imlygic®).

In some embodiments, the immunotherapy is a cancer vaccine. In some embodiments, the cancer vaccine is a human papillomavirus (HPV) vaccine. In some embodiments, the HPV vaccine is Gardasil®, Gardasil9@ or Cervarix®. In some embodiments, the cancer vaccine is a hepatitis B virus (HBV) vaccine. In some embodiments, the HBV vaccine is Engerix-B®, Recombivax HB® or GI-13020 (Tarmogen®). In some embodiments, the cancer vaccine is Twinrix® or Pediarix®. In some embodiments, the cancer vaccine is BiovaxID®, Oncophage®, GVAX, ADXS11-001, ALVAC-CEA, PROSTVAC®, Rindopepimut®, CimaVax-EGF, lapuleucel-T (APC8024; Neuvenge™), GRNVAC1, GRNVAC2, GRN-1201, hepcortespenlisimut-L (Hepko-V5), DCVAX®, SCIB1, BMT CTN 1401, PrCa VBIR, PANVAC, ProstAtak®, DPX-Survivac, or viagenpumatucel-L (HS-110).

In some embodiments, the immunotherapy is a peptide vaccine. In some embodiments, the peptide vaccine is nelipepimut-S(E75) (NeuVax™), IMA901, or SurVaxM (SVN53-67).

In some embodiments, the cancer vaccine is an immunogenic personal neoantigen vaccine (see, e.g., Ott et al. (2017) Nature 547: 217-221; Sahin et al. (2017) Nature 547: 222-226). In some embodiments, the cancer vaccine is RGSH4K, or NEO-PV-01. In some embodiments, the cancer vaccine is a DNA-based vaccine. In some embodiments, the DNA-based vaccine is a mammaglobin-A DNA vaccine (see, e.g., Kim et al. (2016) OncoImmunology 5(2): e1069940).

In some embodiments, immune-targeted agents are selected from aldesleukin, interferon alfa-2b, ipilimumab, lambrolizumab, nivolumab, prednisone, and sipuleucel-T.

Non-limiting examples of radiotherapy include radioiodide therapy, external-beam radiation, and radium 223 therapy.

Additional kinase inhibitors include those described in, for example, U.S. Pat. Nos. 7,514,446; 7,863,289; 8,026,247; 8,501,756; 8,552,002; 8,815,901; 8,912,204; 9,260,437; 9,273,051; U.S. Publication No. US 2015/0018336; International Publication No. WO 2007/002325; WO 2007/002433; WO 2008/080001; WO 2008/079906; WO 2008/079903; WO 2008/079909; WO 2008/080015; WO 2009/007748; WO 2009/012283; WO 2009/143018; WO 2009/143024; WO 2009/014637; 2009/152083; WO 2010/111527; WO 2012/109075; WO 2014/194127; WO 2015/112806; WO 2007/110344; WO 2009/071480; WO 2009/118411; WO 2010/031816; WO 2010/145998; WO 2011/092120; WO 2012/101032; WO 2012/139930; WO 2012/143248; WO 2012/152763; WO 2013/014039; WO 2013/102059; WO 2013/050448; WO 2013/050446; WO 2014/019908; WO 2014/072220; WO 2014/184069; WO 2016/075224; WO 2016/081450; WO 2016/022569; WO 2016/011141; WO 2016/011144; WO 2016/011147; WO 2015/191667; WO 2012/101029; WO 2012/113774; WO 2015/191666; WO 2015/161277; WO 2015/161274; WO 2015/108992; WO 2015/061572; WO 2015/058129; WO 2015/057873; WO 2015/017528; WO/2015/017533; WO 2014/160521; and WO 2014/011900, each of which is hereby incorporated by reference in its entirety.

Provided herein are methods of treating a subject having a cancer (e.g., any of the cancers described herein) and previously administered a multi-kinase inhibitor (MKI) or a target-specific kinase inhibitor (e.g., a BRAF inhibitor, an EGFR inhibitor, a MEK inhibitor, an ALK inhibitor, a ROS1 inhibitor, a MET inhibitor, an aromatase inhibitor, a RAF inhibitor, a RET inhibitor, or a RAS inhibitor) (e.g., as a monotherapy) that include: administering to the subject (i) an effective dose of a compound of Formula (I), Formula (II), and/or Formula (III) (e.g., Formula (I)), or a pharmaceutically acceptable salt thereof as a monotherapy, or (ii) an effective dose of a compound of Formula (I), Formula (II), and/or Formula (III) (e.g., Formula (I)), or a pharmaceutically acceptable salt thereof, and an effective dose of the previously administered MKI or the previously administered target-specific kinase inhibitor.

Also provided herein is a method of inhibiting mammalian cell proliferation, in vitro or in vivo, the method comprising contacting a mammalian cell with an effective amount of a compound of Formula (I), Formula (II), and/or Formula (III) (e.g., Formula (I)), or a pharmaceutically acceptable salt thereof, or a pharmaceutical composition thereof as defined herein.

Some embodiments provide a method of treating an autoimmune disorder in a subject in need thereof, comprising administering a therapeutically effective amount of a compound of Formula (I), or a pharmaceutically acceptable salt thereof, to a subject in need thereof. Some embodiments provide a method of treating an autoimmune disorder comprising administering a therapeutically effective amount of a compound of Formula (II), or a pharmaceutically acceptable salt thereof, to a subject in need thereof. In some embodiments, the autoimmune disorder is selected from SLE, type 1 diabetes, rheumatoid arthritis, psoriatic arthritis, psoriasis, multiple sclerosis, inflammatory bowel disease (e.g., Crohn's disease or ulcerative colitis), Addison's disease, Graves' disease, Sjogren's syndrome, thyroiditis (e.g., Hashimoto's thyroiditis), Myasthenia gravis, autoimmune vasculitis, pernicious anemia, or celiac disease. In some embodiments, the autoimmune disorder is SLE. In some embodiments, treating SLE comprises treating joint pain, heart disease, kidney disease, and photosensitivity in the subject. In some embodiments, treating the autoimmune disorder comprises antagonizing STING, as described herein. In some embodiments, treating the autoimmune disorder comprises reducing expression of IFN-β in the subject, as described herein. In some embodiments, treating the autoimmune disorder comprises reducing expression of one or more cytokines (e.g., two or more, five or more, ten or more, for example up to twenty or fifty cytokines. In some embodiments, the one or more cytokines are induced by IRF3. In some embodiments, the one or more cytokines are induced by NF-κB. In some embodiments, the one or more cytokines are induced by IRF3 and NF-κB. In some embodiments, treating the subject comprises reducing expression of STAT1, STAT2, Cig5, G1P3, IRF7, IFIT4, Ly6E, MX1, OAS3 and IFI27 in the subject. In some embodiments, treating the subject comprises reducing expression of TOR1B, C1orf29, FAM3B, OAS3, USP18, OAS1, Siglec-1, GBP5, IFIT5, IFIT2, IFRG28, IFIT1, PRKR (EIF2AK1), IL1RN, OASL, OAS1, LGALS3BP, OASL, IFIH1 (MDA5), ZBP1, C1QB, CEB1, GBP1, BST2, IFI44, IFI27, GBP2, EPSTIl, CARD15, IFI35, SOCS1, TAP1, XAF1, SP110, OAS2, STAT1, ABCA1, IFIT4, PLSCR1, Cig5, ISG95, STAT2, RIG-I (DDX58), MX2, LGP2, IRF7, ADD45B, SCOTIN, PARP9 (BAL), MT2A, NT5C3 (PN-1), MX1, STAT1, ADAR, TRIM22, G1P2, SERPING1, STAT1, NUB1 (NYREN18), ISG20, LY6E, G1P3, and/or IFITM1 in the subject. In some embodiments, treating the subject comprises reducing expression of STAT1, STAT2, Cig5, G1P3, IRF7, IFIT4, Ly6E, MX1, OAS3 and/or IFI27 in the subject.

In some embodiments, treating the autoimmune disorder comprises reducing anti-DNA antibody production. In some embodiments, anti-DNA antibody production is reduced by about 10% to about 99%, for example, about 10% to about 40%, about 25% to about 50%, about 35% to about 75%, about 50% to about 80%, or about 70% to about 99%. In some embodiments, anti-DNA antibody production is reduced by about 10%, about 20%, about 30%, about 40%, about 50%, about 60%, about 70%, about 80%, about 90%, or about 99%.

In some embodiments, treating the autoimmune disorder comprises reducing inflammation in the subject. In some embodiments, the inflammation is reduced by about 10% to about 99%, for example, about 10% to about 40%, about 25% to about 50%, about 35% to about 75%, about 50% to about 80%, or about 70% to about 99%. In some embodiments, inflammation is reduced by about 10%, about 20%, about 30%, about 40%, about 50%, about 60%, about 70%, about 80%, about 90%, or about 99%. In some embodiments, the reduction in inflammation is determined by measuring one or more biomarkers associated with inflammation. See, e.g., Brenner, et al., Cancer Epidemiol Biomarkers Prev. 2014 September; 23(9): 1729-1751; Zakynthinos and Pappa, J. Cardiol., 53(3), 317-333 (2009); Liu, et al., Nat. Immunol., 18; 1175-1180 (2017); Roemer, et al., Arthrit. Rheum. 71(2) 238-243 (2019), each of which is incorporated by reference in its entirety.

In some embodiments, the subject also has a mutation in the TREX1 gene. In some embodiments, the autoimmune disorder is associated with the mutation in the TREX1 gene in the subject.

It is normal practice to use a combination of different forms of treatment to treat a subject with an autoimmune disorder. The other component(s) of such conjoint treatment or therapy in addition to compositions provided herein (e.g., the compounds of Formula (I), Formula (II), and/or Formula (III) (e.g., Formula (I)), or pharmaceutically acceptable salts thereof) may be, for example, anti-inflammatory drugs, cytotoxic chemotherapeutic drugs, immunosuppressants, kidney support, anti-rheumatic drugs, monoclonal antibodies, and avoiding sun exposure.

In some embodiments of any of the methods described herein, the compound of Formula (I), Formula (II), and/or Formula (III) (e.g., Formula (I)), or a pharmaceutically acceptable salt thereof, is administered in combination with an effective amount of at least one anti-inflammatory drug selected from steroids (e.g., corticosteroids, hydrocortisone, prednisone, triamcinolone, betamethasone, dexamethasone, Prednisolone, methylprednisolone), ibuprofen, naproxen, aspirin, diclofenac, meloxicam, and tolmetin. The at least one anti-inflammatory drug may be a topical drug or treatment. Topical drugs or treatments include, but are not limited to, triamcinolone and fluocinolone.

In some embodiments of any of the methods described herein, the compound of Formula (I), Formula (II), and/or Formula (III) (e.g., Formula (I)), or a pharmaceutically acceptable salt thereof, is administered in combination with an effective amount of at least one cytotoxic chemotherapeutic drug selected from arsenic trioxide, bleomycin, cabazitaxel, capecitabine, carboplatin, cisplatin, cyclophosphamide, cytarabine, dacarbazine, dauno-rubicin, docetaxel, doxorubicin, etoposide, fluorouracil, gemcitabine, irinotecan, lomustine, methotrexate, mitomycin C, oxaliplatin, paclitaxel, pemetrexed, temozolomide, and vincristine.

In some embodiments of any of the methods described herein, the compound of Formula (I), Formula (II), and/or Formula (III) (e.g., Formula (I)), or a pharmaceutically acceptable salt thereof, is administered in combination with an effective amount of at least one immunosuppressant selected from azathioprine, methotrexate, mycophenolate, imuran, azathioprine, mycophenolate mofetil, tacrolimus, sirolimus, everolimus, and interferons.

In some embodiments of any of the methods described herein, the compound of Formula (I), Formula (II), and/or Formula (III) (e.g., Formula (I)), or a pharmaceutically acceptable salt thereof, is administered in combination with an effective amount of at least one anti-rheumatic drug selected from hydroxychloroquine, celecoxib, abatacept, adalimumab, anakinra, apremilast, baricitinib, certolizumab pegol, ciclosporin (Cyclosporin A), D-penicillamine, etanercept, filgotinib, golimumab, infliximab, leflunomide, methotrexate, minocycline, rituximab, sarilumab, secukinumab, sulfasalazine, tocilizumab, tofacitinib, and ustekinumab.

In some embodiments of any of the methods described herein, the compound of Formula (I), Formula (II), and/or Formula (III) (e.g., Formula (I)), or a pharmaceutically acceptable salt thereof, is administered in combination with an effective amount of at least one monoclonal antibody selected from abciximab, alefacept, alemtuzumab, basiliximab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivizumab, panitumumab, pembrolizumab, tocilizumab, trastuzumab, secukinumab, ustekinumab, belimumab, rituximab, infliximab, and adalimumab.

Some embodiments provide a method of treating Aicardi-Goutieres Syndrome comprising administering a therapeutically effective amount of a compound of Formula (I), Formula (II), and/or Formula (III) (e.g., Formula (I)), or a pharmaceutically acceptable salt thereof, to a subject in need thereof. In some embodiments, the subject also has a mutation in the TREX1 gene. In some embodiments, the Aicardi-Goutieres Syndrome is associated with the mutation in the TREX1 gene in the subject.

Some embodiments provide a method of treating embryonic lethality polyarthritis comprising administering a therapeutically effective amount of a compound of Formula (I), Formula (II), and/or Formula (III) (e.g., Formula (I)), or a pharmaceutically acceptable salt thereof, to a subject in need thereof. In some embodiments, the subject has a Dnase II genetic deficiency. In some embodiments, the embryonic lethality polyarthritis is associated with a Dnase II genetic deficiency in the subject.

Some embodiments provide a method of treating Type I interferon perinatal lethality comprising administering a therapeutically effective amount of a compound of Formula (I), Formula (II), and/or Formula (III) (e.g., Formula (I)), or a pharmaceutically acceptable salt thereof, to a subject in need thereof. In some embodiments, the subject has a genetic deficiency in RNaseH2. In some embodiments, the Type I interferon perinatal lethality is associated with a genetic deficiency in RNaseH2 in the subject.

Some embodiments provide a method of treating inflammation comprising administering a therapeutically effective amount of a compound of Formula (I), Formula (II), and/or Formula (III) (e.g., Formula (I)), or a pharmaceutically acceptable salt thereof, to a subject in need thereof.

Some embodiments provide a method of treating inflammation comprising administering a therapeutically effective amount of a compound of Formula (I), Formula (II), and/or Formula (III) (e.g., Formula (I)), or a pharmaceutically acceptable salt thereof, to a subject in need thereof.

Some embodiments provide a method of treating a viral infection comprising administering a therapeutically effective amount of a compound of Formula (I), Formula (II), and/or Formula (III) (e.g., Formula (I)), or a pharmaceutically acceptable salt thereof, to a subject in need thereof. In some embodiments, the viral infection is caused by a DNA or RNA virus. In some embodiments, the viral infection is caused by a DNA virus. In some embodiments, the viral infection is caused by an RNA virus. In some embodiments, the viral infection is caused by a coronavirus. In some embodiments, the viral infection is caused by SARS-CoV-2.

### Pharmaceutical Compositions

When employed as pharmaceuticals, compounds of Formula (I), Formula (II), and/or Formula (III) (e.g., Formula (I)), including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I), Formula (II), and/or Formula (III) (e.g., Formula (I)) or pharmaceutically acceptable salt thereof, in combination with one or more pharmaceutically acceptable excipients. For example, a pharmaceutical composition prepared using a compound of Formula (I), Formula (II), and/or Formula (III) (e.g., Formula (I)) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I), Formula (II), and/or Formula (III) (e.g., Formula (I)) or a pharmaceutically acceptable salt thereof with a pharmaceutically acceptable carrier. Pharmaceutical compositions containing a compound of Formula (I), Formula (II), and/or Formula (III) (e.g., Formula (I)) or a pharmaceutically acceptable salt thereof as the active ingredient can be prepared by intimately mixing the compound of Formula (I), Formula (II), and/or Formula (III) (e.g., Formula (I)), or a pharmaceutically acceptable salt thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts.

Provided herein are pharmaceutical kits useful, for example, in the treatment of STING-associated diseases or disorders, such as cancer, which include one or more containers containing a pharmaceutical composition comprising an effective amount of a compound provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “pharmaceutically acceptable salt” indicates that the salt is compatible chemically and/or toxicologically with the other ingredients comprising a formulation and/or the subject being treated therewith.

The term “partial agonist” or “partially agonizing”, as used herein, refers to a property of a compound to bind to and activate a given receptor or protein (e.g., STING), but have partial efficacy or activity at the receptor at any concentration relative to a full agonist. In some embodiments, partial agonism of STING may be associated with a net negative effect, resulting in suppression of the STING signal.

The term “full agonist”, as used herein, refers to a property of a compound to bind to and activate a given receptor or protein (e.g., STING), with the maximum response that an agonist can elicit at the receptor (e.g., with the response that an endogenous ligand can elicit at the receptor).

The term “halogen,” refers to —F, —Cl, —Br and —I.

The term “pseudohalogen,” as used herein, refers to polyatomic analogues of halogens, which possess similar chemistry (e.g., stereoelectronic characteristics and/or bioisosterism) to halogens. Pseudohalogens include, but are not limited to cyano, isocyano, thiocyano, isothiocyano, —OH, —SH, azide, and trifluoromethanesulfonate.

The term “alkyl” as used herein refers to saturated linear or branched-chain monovalent hydrocarbon radicals of the indicated number of carbon atoms; and “alkylene” refers to saturated linear or branched-chain monovalent hydrocarbon diradicals of the indicated number of carbon atoms. Alkyl groups can be straight chain or branched. Examples include, but are not limited to, methyl, ethyl, 1-propyl, isopropyl, 1-butyl, isobutyl, sec-butyl, tert-butyl, 2-methyl-2-propyl, pentyl, neopentyl, and hexyl. The term “heteroalkylene” refers to an alkylene wherein 1-4 carbon atoms are independently replaced by a heteroatom selected from O, NH, or S.

The term “alkenylene” as used herein refers to an acyclic hydrocarbon diradical that may be a straight chain or branched chain having one or more carbon-carbon double bonds. The alkenylene moiety contains the indicated number of carbon atoms. For example, C2-C10 indicates that the group may have from 2 to 10 (inclusive) carbon atoms in it.

The term “alkynylene” refers to an acyclic hydrocarbon chain that may be a straight chain or branched chain diradical having one or more carbon-carbon triple bonds. The alkynyl moiety contains the indicated number of carbon atoms. For example, C2-C10 indicates that the group may have from 2 to 10 (inclusive) carbon atoms in it. As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C6-C14 aryl group, a C6-C10 aryl group, or a C6 aryl group. Examples of aryl groups include, but are not limited to, benzene, naphthalene, and azulene.

As used herein, “heteroaryl,” refers to a monocyclic, bicyclic or tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond.

Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline, and triazine.

As used herein, “heterocyclyl” and “heterocycle” refer to three-, four-, five-, six-, seven-, eight-, nine-, ten-, up to 18-membered monocyclic, bicyclic, or tricyclic ring system that is not fully aromatic wherein carbon atoms together with from 1 to 5 heteroatoms constitute said ring system. A heterocycle may optionally contain one or more elements of unsaturation. The heteroatom(s) is an element other than carbon including, but not limited to, oxygen, sulfur, and nitrogen. A heterocycle may further contain one or more carbonyl or thiocarbonyl functionalities, so as to make the definition include oxo-containing systems and thio-containing systems such as lactams, lactones, cyclic imides, cyclic thioimides, and cyclic carbamates. When composed of two or more rings, the rings may be joined together in a fused, bridged, or spirocyclic fashion. Additionally, any nitrogens in a heterocycle may be quaternized. Heterocyclyl groups may be unsubstituted or substituted. Examples of such “heterocyclyl” groups include, but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone and their benzo-fused analogs (e.g., benzimidazolidinone, tetrahydroquinoline and 3,4-methylenedioxyphenyl).

The term “compound,” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The term “tautomer” as used herein refers to compounds whose structures differ markedly in arrangement of atoms, but which interconvert according to, e.g., a facile and rapid equilibrium. It is to be understood that compounds provided herein may be depicted as different tautomers, and when compounds have tautomeric forms, all tautomeric forms are intended to be within the scope of the invention, and the naming of the compounds does not exclude any tautomer.

It will be appreciated that certain compounds provided herein may contain one or more centers of asymmetry and may therefore be prepared and isolated in a mixture of isomers such as a racemic mixture, or in an enantiomerically pure form.

### Examples

Abbreviations

| | |
|---|---|
| DCM | dichloromethane |
| DMAP | 4-Dimethylaminopyridine |
| DMF | N,N-dimethylformamide |
| DMSO | dimethylsulfoxide |
| EDC | 1-Ethyl-3-(3-dimethylaminopropyl) |
| | carbodiimide |
| HATU | 1-[Bis(dimethylamino)methylene]-1H- |
| | 1,2,3-triazolo[4,5-b]pyridinium 3-oxide |
| | hexafluorophosphate |
| HRMS | high resolution mass spectrometry |
| KOH | potassium hydroxide |
| LAH | lithium aluminum hydride |
| MeOH | methanol |
| n-BuLi | n-butyllithium |
| NMR | nuclear magnetic resonance |
| TEA | triethylamine |
| THE | tetrahydrofuran |
| TLC | thin layer chromatography |

### Example 1: Experimental Procedures

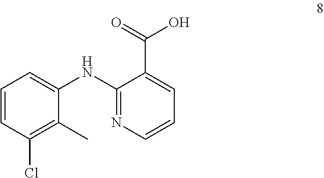 [SMILES: Cc1c(Cl)cccc1Nc1ncccc1C(=O)O]

A mixture of 3-chloro-2-methylaniline (1.67 ml, 14.0 mmol), 2-chloronicotinic acid (2.00 g, 12.7 mmol), and p-toluenesulfonic acid monohydrate (0.193 g, 1.02 mmol) in water (6 mL) was heated at 100° C. for 24 hours. KOH (1.78 g, 31.7 mmol) in water (4 mL) was added and the mixture was cooled to 50° C., treated with decolorizing charcoal, and filtered. The pH was adjusted to 5 with sulfuric acid resulting in precipitation of a white solid. The precipitate was recrystallized in methanol, washed with water, and dried under vacuum. ¹H NMR of the re-crystallized solid matched the literature values for the title compound 8 (2.40 g, 72.1%).

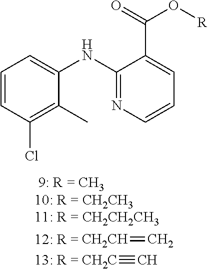

In a round bottom flask, compound 8 (100 mg, 0.381 mmol) in DCM (Volume: 4 mL) was brought to 0° C. A mixture of EDC (0.146 g, 0.761 mmol), DMAP (4.65 mg, 0.038 μmol), and excess of the appropriate alcohol was added to the reaction while stirring at 0° C. for 1 h before being brought to room temperature and stirred overnight. The reaction was quenched with water and organic layer was washed with saturated NaHCO₃ and brine. The resulting oil was purified via column chromatography (5% MeOH in DCM) to yield compounds 9-13.

Compound 9: Yield 40.1% ¹H NMR (600 MHz, chloroform-d) δ 9.84-9.92 (m, 1H), 8.24-8.28 (m, 1H), 8.15-8.20 (m, 1H), 7.81-7.85 (m, 1H), 7.06-7.11 (m, 2H), 6.63-6.67 (m, 1H), 3.88 (s, 3H), 2.34 (s, 3H), ppm. ¹³C NMR (151 MHz, chloroform-d) δ 168.1, 156.47, 153.4, 140.3, 139.2, 134.9, 128.9, 126.6, 124.9, 122.0, 113.4, 105.0-108.7, 52.4, 15.1 ppm.

Compound 10: Yield 38.0% ¹H NMR (600 MHz, chloroform-d) δ 9.87-9.96 (m, 1H), 8.22-8.25 (m, 1H), 8.14-8.20 (m, 1H), 7.83-7.88 (m, 1H), 7.06 (s, 2H), 6.59-6.64 (m, 1H), 4.31 (d, J=6.90 Hz, 2H), 2.32 (s, 3H), 1.33 (t, J=7.08 Hz, 3H), ppm. ¹³C NMR (151 MHz, chloroform-d) δ 167.6, 156.5, 153.2, 140.2, 139.4, 134.8, 128.6, 126.6, 124.7, 121.8, 113.4, 107.4, 61.4, 15.1, 14.3, ppm.

Compound 11: Yield 20.2% ¹H NMR (400 MHz, chloroform-d) δ 9.86-9.98 (m, 1H), 8.24-8.28 (m, 1H), 8.17-8.23 (m, 1H), 7.81-7.87 (m, 1H), 7.02-7.14 (m, 3H), 6.63-6.68 (m, 1H), 4.21-4.26 (m, 2H), 2.34 (s, 3H), 1.71-1.80 (m, 2H), 0.98 (t, J=7.50 Hz, 3H), ppm. ¹³C NMR (151 MHz, chloroform-d) δ 167.7, 156.5, 153.1, 140.3, 134.9, 128.8, 126.6, 124.9, 121.9, 113.4, 107.5, 66.9, 22.1, 15.1, 10.5 ppm.

Compound 12: Yield 27.3% ¹H NMR (600 MHz, chloroform-d) δ 9.85 (br s, 1H), 8.13-8.30 (m, 2H), 7.82 (dd, J=7.08, 1.63 Hz, 1H), 6.99-7.12 (m, 2H), 6.63 (dd, J=7.63, 4.72 Hz, 1H), 5.88-6.06 (m, 1H), 5.35 (dd, J=17.26, 0.91 Hz, 1H), 5.25 (d, J=10.17 Hz, 1H), 4.76 (d, J=5.45 Hz, 2H), 2.32 (s, 3H), ppm. ¹³C NMR (151 MHz, chloroform-d) δ 167.2, 156.5, 153.4, 140.3, 139.2, 134.8, 131.8, 128.9, 126.6, 124.9, 122.0, 118.8, 113.4, 107.1, 65.8, 15.1, ppm.

Compound 13: Yield 37.8% ¹H NMR (600 MHz, chloroform-d) δ ppm 9.73 (br s, 1H), 8.19-8.31 (m, 2H), 7.75-7.80 (m, 1H), 7.03-7.14 (m, 2H), 6.61-6.70 (m, 1H), 4.87 (d, J=2.18 Hz, 2H), 2.44-2.52 (m, 1H), 2.32 (s, 3H).

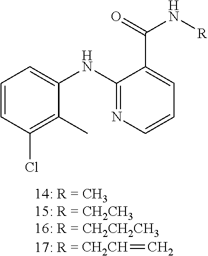

In a round bottom flask, compound 8 (500 mg, 1.90 mmol), HATU (1.45 g, 3.81 mmol), tri-ethylamine (0.531 mL, 3.81 mmol), and excess of the appropriate amine was dissolved in DMF and left to stir overnight. Once completed, the reaction mixture was quenched with 2N HCl and organic material was extracted with ethyl acetate. The crude organic product was concentrated under vacuum and purified via column chromatography (5% MeOH in DCM) to yield compounds 14-17.

Compound 14: Yield (48.0%)¹H NMR (600 MHz, chloroform-d) δ 10.18 (br s, 1H), 8.14-8.22 (m, 1H), 7.79-7.85 (m, 1H), 7.57-7.61 (m, 1H), 7.01-7.09 (m, 2H), 6.57-6.63 (m, 1H), 6.12-6.25 (m, 1H), 2.93 (d, J=4.72 Hz, 3H), 2.34 (s, 3H), ppm. ¹³C NMR (151 MHz, chloroform-d) δ 168.8, 155.5, 151.4, 139.6, 135.3, 134.7, 128.2, 126.4, 124.2, 121.0, 113.1, 111.3, 26.8, 15.1, ppm.

Compound 15: Yield (64.1%)¹H NMR (600 MHz, chloroform-d) δ 10.24-10.33 (m, 1H), 8.27 (dd, J=4.90, 1.64 Hz, 1H), 7.87-7.93 (m, 1H), 7.68-7.74 (m, 1H), 7.11-7.17 (m, 2H), 6.68-6.73 (m, 1H), 6.19-6.29 (m, 1H), 3.47-3.54 (m, 2H), 2.39-2.44 (m, 3H), 1.26-1.31 (m, 3H), ppm. ¹³C NMR (101 MHz, chloroform-d) δ 168.1, 155.6, 151.3, 139.6, 135.33, 134.8, 128.2, 126.5, 124.3, 121.0, 113.0, 111.5, 35.01, 14.8 ppm.

Compound 16: Yield (17.3%)¹H NMR (600 MHz, chloroform-d) δ 10.11-10.19 (m, 1H), 8.15-8.19 (m, 1H), 7.80-7.86 (m, 1H), 7.58-7.62 (m, 1H), 7.01-7.07 (m, 2H), 6.58-6.62 (m, 1H), 6.18-6.27 (m, 1H), 3.29-3.34 (m, 2H), 2.33 (s, 3H), 1.54-1.60 (m, 2H), 0.89-0.94 (m, 3H), ppm. 13C NMR (151 MHz, chloroform-d) δ 168.19, 155.51, 151.26, 139.65, 135.38, 134.76, 128.13, 126.48, 124.19, 120.93, 113.14, 111.62, 41.79, 22.83, 15.11, 11.50 ppm.

Compound 17: Yield (35.5%)¹H NMR (600 MHz, chloroform-d) δ 2.31 (s, 3H), 3.96 (t, J=5.63 Hz, 3H), 5.09-5.14 (m, 1H), 5.14-5.22 (m, 1H), 5.78-5.88 (m, 1H), 6.34 (br s, 1H), 6.57 (dd, J=7.63, 5.09 Hz, 1H), 7.02 (d, J=4.72 Hz, 2H), 7.59 (dd, J=7.63, 1.45 Hz, 1H), 7.77-7.83 (m, 1H), 8.15 (dd, J=4.90, 1.63 Hz, 1H), 10.12 (s, 1H), ppm. ¹³C NMR (151 MHz, chloroform-d) δ 15.1, 42.3, 111.1, 113.1, 117.1, 121.1, 124.3, 126.4, 128.3, 133.6, 134.7, 135.4, 139.6, 151.4, 155.6, 168.0 ppm.

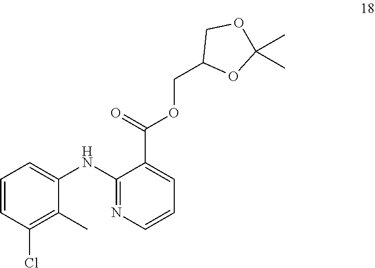 [SMILES: Cc1c(Cl)cccc1Nc1ncccc1C(=O)OCC1COC(C)(C)O1]

Compound 18 was prepared using an analogous procedure to that of compounds 9-13. Product was brought to the next step without further purification. Yield 23.1%.

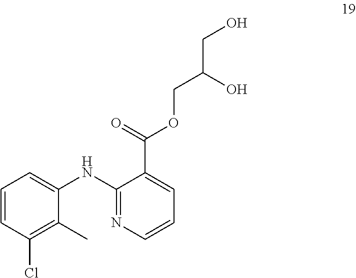 [SMILES: Cc1c(Cl)cccc1Nc1ncccc1C(=O)OCC(O)CO]

In a round bottom flask compound 18 (100 mg) was added to a 1:1 mixture of THF:HCl (2 M). The reaction mixture was left to stir for 30 minutes and reaction completion was moni-tored via TLC. When the reaction reached completion, ethyl acetate and water was added to the reaction mixture. The organic layer was separated, dried with Na₂SO₄, and solvent was removed under reduced pressure to yield compound 19 as an oil. The crude oil was purified via column chromatography (1:2 hexanes:ethyl acetate). ¹H NMR (600 MHz, chloroform-d) δ 9.75-9.83 (m, 1H), 8.25-8.30 (m, 1H), 8.16-8.23 (m, 1H), 7.73-7.78 (m, 1H), 7.05-7.14 (m, 2H), 6.62-6.69 (m, 1H), 4.30-4.40 (m, 2H), 3.97-4.04 (m, 1H), 3.69-3.76 (m, 1H), 3.57-3.64 (m, 1H), 2.31 (s, 3H) ppm.

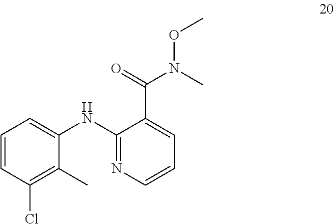 [SMILES: CON(C)C(=O)c1cccnc1Nc1cccc(Cl)c1C]

To a solution of compound 8 (0.65 g 2.47 mmol) in anhydrous DCM (18 mL) was added oxalyl chloride (0.260 mL, 2.97 mmol) and anhydrous DMF (3-5 drops) under argon atmosphere. After 1 hour, the reaction mixture was concentrated in vacuo to dryness. N, O dimethylhydroxylamine hydrochloride (0.362 g, 3.71 mmol) was suspended in anhydrous DCM (18 mL) and anhydrous TEA (0.862 mL, 6.19 mmol) and cooled to 0° C. under argon atmosphere. The newly formed acid chloride was in DCM (18 mL) and slowly added to the cooled solution under argon atmosphere. The mixture was then warmed to rt, stirred for 4 hours, then washed with saturated NH₄C1 solution, water, and dried over MgSO₄. The crude reaction mixture was passed through a silica plug (1:4 diethyl ether:DCM) and concentrated under vacuum to give compound 20 as a white solid. This compound was brought to the next step without further purification.

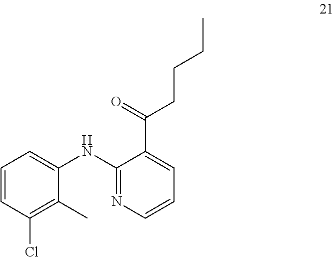 [SMILES: CCCCC(=O)c1cccnc1Nc1cccc(Cl)c1C]

In a round bottom flask, compound 20 (0.150 g, 0.491 mmol) was dissolved in THF (2 mL) and brought to −78° C. while stirring, followed by the dropwise addition of n-BuLi (0.307 ml, 0.491 mmol). The reaction was left for 1 hour then brought to room temperature. The reaction was quenched with NH₄C1 and extracted with ethyl acetate. The crude organic material was purified via column chromatography (1:4 diethyl ether:DCM) to yield compound 21 (0.084 g, 56.5%). HRMS m/z: [M+H]⁺ calc'd for C₁₇H₂₀ClN₂O 303.1259; Found 303.1269 ppm.

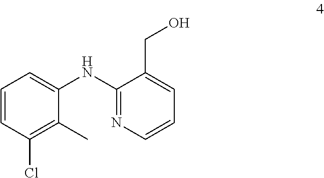 [SMILES: Cc1c(Cl)cccc1Nc1ncccc1CO]

In a round bottom flask, compound 8 (2 g, 7.61 mmol) was dissolved in anhydrous THE (10 mL) and cooled to 0° C. under argon. A 1 M solution of LAH in THE (19.0 mL, 19.0 mmol) was added dropwise. After the addition was complete, the solution was allowed to warm to room temperature and stirred for an additional 1 hour. The solution was cooled to 0° C. and quenched by the addition of ethyl acetate (20 mL) followed by water. (20 mL). The mixture was diluted with ethyl acetate and filtered through a pad of celite. The combined filtrates were dried over sodium sulfate and concentrated in vacuo. The extract was pure compound 4 (1.76 g, 93.0%). ¹H NMR (600 MHz, chloroform-d) δ 7.91-7.97 (m, 1H), 7.59-7.64 (m, 1H), 7.51-7.57 (m, 1H), 7.14-7.17 (m, 1H), 6.94-7.01 (m, 2H), 6.53-6.59 (m, 1H), 4.41 (s, 2H), 2.16 (s, 3H) ppm. ¹³C NMR (151 MHz, chloroform-d) δ 154.1, 145.9, 139.2, 135.6, 133.8, 126.0, 125.6, 122.6, 119.7, 118.8, 113.6, 62.4, 13.6 ppm.

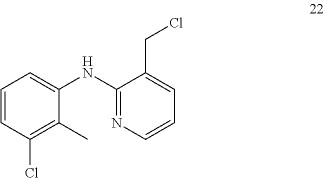 [SMILES: Cc1c(Cl)cccc1Nc1ncccc1CCl]

In a round bottom flask, compound 4 (0.787 g, 3.16 mmol) was dissolved in dry DCM then cooled to 0° C. Triethylamine (1.10 ml, 7.91 mmol) was then added via syringe, followed by the addition of triphosgene (0.939 g, 3.16 mmol). The mixture was stirred at 0° C. for 3 minutes then allowed to warm to room temperature. The reaction was checked for completion by TLC after 3 hours. When completed, the reaction was quenched with saturated aqueous solution of NaHCO₃, yielding a biphasic mixture. The organic extract was separated, dried with MgSO₄, and concentrated under vacuum. The resulting crude oil was purified via column chromatography (1:1 hexanes:ethyl acetate) to yield compound 22 (0.785, 93.0%)

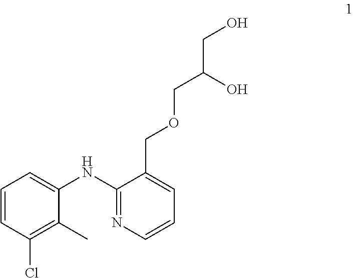 [SMILES: Cc1c(Cl)cccc1Nc1ncccc1COCC(O)CO]

To a solution of solketal (0.388 mL, 3.12 mmol) and compound 22 (0.100 g, 0.374 mmol) in Toluene (2 mL) was added powdered KOH (0.175 g, 3.12 mmol) and the mixture was re-fluxed with stirring for 16 h. Reaction mixture was quenched with water and extracted with ethyl acetate. The organic layer was dried with MgSO₄ and concentrated under vacuum. Crude extract was purified via column chromatography (1:1 hexanes: ethyl acetate) to yield compound 1 (0.069, 61.0%). HRMS m/z: [M+H]⁺ calc'd for C₁₆H₂₀ClN₂O₃ 323.1157; Found 323.1157 ppm.

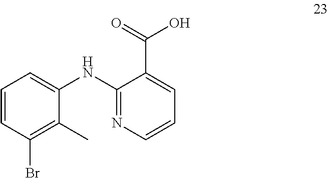 [SMILES: Cc1c(Br)cccc1Nc1ncccc1C(=O)O]

Compound 23 was prepared using a procedure analogous to that of compound 8. Yield 86.0%. ¹H NMR (600 MHz, DMSO-d6) δ 10.28 (s, 1H), 8.32-8.34 (m, 1H), 8.24-8.27 (m, 1H), 8.09-8.13 (m, 1H), 7.32 (s, 1H), 6.85-6.88 (m, 1H), 2.36 (s, 3H) ppm. ¹³C NMR (151 MHz, DMSO-d6) δ ppm 169.6, 153.1, 141.0, 140.0, 127.8, 127.5, 125.0, 122.5, 114.5, 108.3, 18.4 ppm.

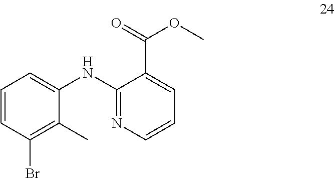 [SMILES: COC(=O)c1cccnc1Nc1cccc(Br)c1C]

Compound 24 was prepared using a procedure analogous to that of compound 9. 56.1%. ¹H NMR (600 MHz, chloroform-d) δ 2.38 (s, 4H), 3.87 (s, 4H), 6.61-6.66 (m, 1H), 6.98-7.02 (m, 1H), 7.26-7.30 (m, 1H), 7.83-7.87 (m, 1H), 8.14-8.18 (m, 1H), 8.23-8.26 (m, 1H), 9.84-9.90 (m, 1H) ppm. ¹³C NMR (151 MHz, chloroform-d) δ 167.0, 155.5, 152.3, 139.3, 138.0, 129.5, 127.3, 126.0, 124.5, 121.7, 112.4, 106.0, 51.2, 17.2 ppm

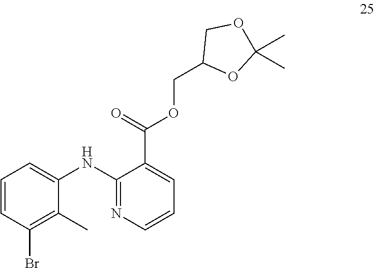 [SMILES: Cc1c(Br)cccc1Nc1ncccc1C(=O)OCC1COC(C)(C)O1]

Compound 25 was prepared using a procedure analogous to that of compounds 9-13. Product was brought to next step without further purification.

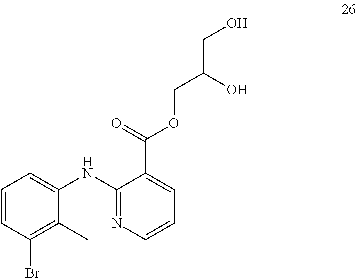 [SMILES: Cc1c(Br)cccc1Nc1ncccc1C(=O)OCC(O)CO]

Compound 26 was prepared using a procedure analogous to that of compound 19. Yield 24.3%. ¹H NMR (600 MHz, chloroform-d) δ 9.75 (s, 1H), 8.21-8.29 (m, 1H), 8.17 (dd, J=7.81, 2.00 Hz, 1H), 7.76 (d, J=7.99 Hz, 1H), 7.30 (d, J=7.99 Hz, 1H), 7.01 (t, J=7.99 Hz, 1H), 6.64 (dd, J=7.99, 4.72 Hz, 1H), 4.29-4.39 (m, 2H), 3.96-4.04 (m, 1H), 3.67-3.74 (m, 1H), 3.56-3.65 (m, 1H), 2.35 (s, 3H) ppm. ¹³C NMR (151 MHz, CHLOROFORM-d) δ 167.71, 156.61, 153.79, 140.40, 138.80, 131.07, 128.66, 127.10, 125.62, 123.16, 113.47, 106.54, 70.14, 65.89, 63.41, 18.35 ppm.

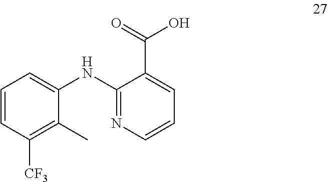 [SMILES: Cc1c(Nc2ncccc2C(=O)O)cccc1C(F)(F)F]

Compound 27 was prepared using a procedure analogous to that of compound 8. Yield 69.3%. ¹H NMR (600 MHz, DMSO-d6) δ 13.52-13.84 (m, 1H), 10.38 (s, 1H), 8.35-8.43 (m, 1H), 8.28 (dd, J=7.63, 1.82 Hz, 1H), 8.13-8.21 (m, 1H), 7.21 (q, J=7.99 Hz, 1H), 6.85-6.92 (m, 3H), 2.19 (s, 3H) ppm. ¹³C NMR (151 MHz, DMSO-d6) δ 169.7, 156.3, 153.1, 141.1, 140.4, 128.7, 128.5, 127.9, 127.1, 126.7, 120.7, 114.8, 108.5, 14.1.

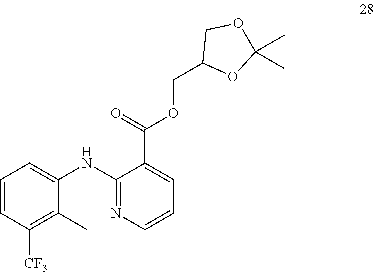 [SMILES: Cc1c(Nc2ncccc2C(=O)OCC2COC(C)(C)O2)cccc1C(F)(F)F]

Compound 28 was prepared using a procedure analogous to that of compounds 9-13. Product was brought to next step without further purification.

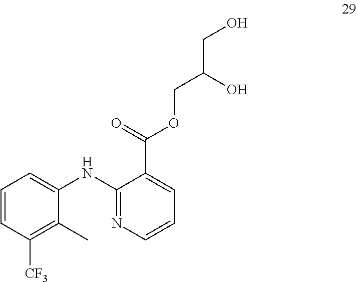 [SMILES: Cc1c(Nc2ncccc2C(=O)OCC(O)CO)cccc1C(F)(F)F]

Compound 29 was prepared using a procedure analogous to that of compound 19. Yield 12.6%. ¹H NMR (600 MHz, chloroform-d) δ 9.69-9.93 (m, 1H), 8.15-8.32 (m, 2H), 8.01-8.14 (m, 1H), 7.32-7.42 (m, 1H), 7.20-7.28 (m, 1H), 6.62-6.74 (m, 1H), 4.31-4.46 (m, 2H), 3.98-4.10 (m, 1H), 3.69-3.82 (m, 1H), 3.58-3.70 (m, 1H), 2.36 (s, 3H) ppm. ¹³C NMR (151 MHz, DMSO-d6) δ 167.8, 156.6, 153.8, 140.4, 139.2, 129.8, 129.4, 127.3, 125.9, 123.6., 121.7, 113.7, 106.7, 70.2, 65.9, 63.4 ppm.

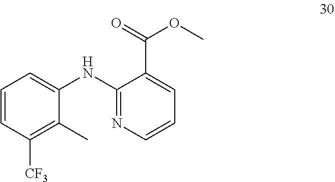 [SMILES: COC(=O)c1cccnc1Nc1cccc(C(F)(F)F)c1C]

Compound 30 was prepared using a procedure analogous to that of compound 9. Yield 60.3%. ¹H NMR (600 MHz, chloroform-d) δ 9.9 (br s, 1H), 8.22-8.26 (m, 1H), 8.14-8.19 (m, 1H), 8.08-8.14 (m, 1H), 7.31-7.36 (m, 1H), 7.19-7.24 (m, 1H), 6.61-6.67 (m, 1H), 3.86 (s, 3H), 2.34-2.39 (m, 3H) ppm. ¹³C NMR (151 MHz, chloroform-d) δ 168.1, 156.4, 153.3, 140.3, 139.4, 129.7, 129.0, 126.9, 125.8, 123.7, 121.3, 113.7, 107.3, 52.3, 14.0 ppm.

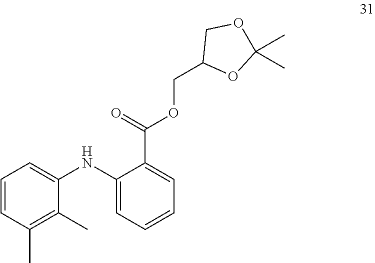 [SMILES: Cc1cccc(Nc2ccccc2C(=O)OCC2COC(C)(C)O2)c1C]

Compound 31 was prepared using a procedure analogous to that of compounds 9-13. Product was brought to next step without further purification.

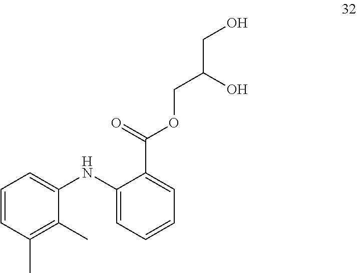 [SMILES: Cc1cccc(Nc2ccccc2C(=O)OCC(O)CO)c1C]

Compound 32 was prepared using a procedure analogous to that of compound 19. Yield 85.0%. ¹H NMR (600 MHz, chloroform-d) δ 9.02-9.13 (m, 1H), 7.82-7.91 (m, 1H), 7.14-7.18 (m, 1H), 7.04-7.08 (m, 1H), 7.00-7.04 (m, 1H), 6.92-6.97 (m, 1H), 6.63-6.67 (m, 1H), 6.55-6.59 (m, 1H), 4.29-4.37 (m, 2H), 3.98-4.04 (m, 1H), 3.69-3.74 (m, 1H), 3.61-3.65 (m, 1H), 2.75-3.16 (m, 1H), 2.35-2.70 (m, 1H), 2.24 (s, 3H), 2.08 (s, 3H) ppm. 13C NMR (151 MHz, chloroform-d) δ ppm 168.9, 149.8, 138.5, 138.3, 134.6, 132.6, 131.5, 127.0, 126.0, 123.3, 116.1, 113.8, 110.1, 70.5, 65.3, 63.5, 20.6, 14.0 ppm.

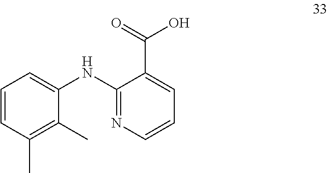 [SMILES: Cc1cccc(Nc2ncccc2C(=O)O)c1C]

Compound 33 was prepared using a procedure analogous to that of compound 8. Yield 77.0%. ¹H NMR (600 MHz, DMSO-d6) δ 10.13 (s, 1H), 8.29 (dd, J=4.72, 2.18 Hz, 1H), 8.21-8.26 (m, 1H), 7.81 (d, J=7.99 Hz, 1H), 7.04-7.08 (m, 1H), 6.94 (d, J=7.63 Hz, 1H), 6.80 (dd, J=7.63, 4.72 Hz, 1H), 2.28-2.31 (m, 3H), 2.16 (s, 3H) ppm. ¹³C NMR (151 MHz, DMSO-d6) δ 169.8, 156.8, 153.2, 141.05, 138.2, 137.1, 128.7, 125.8, 125.7, 121.5, 113.8, 107.7, 20.88, 14.18 ppm.

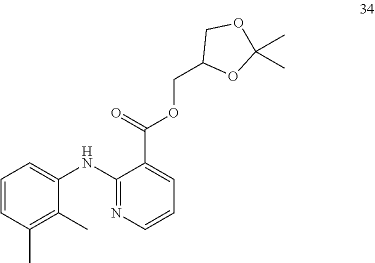 [SMILES: Cc1cccc(Nc2ncccc2C(=O)OCC2COC(C)(C)O2)c1C]

Compound 34 was prepared using a procedure analogous to that of compound 9-13. Product was brought to next step without further purification.

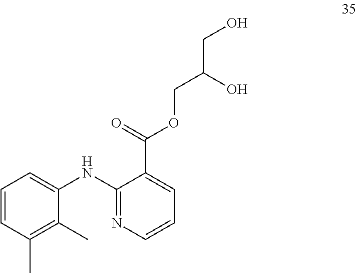 [SMILES: Cc1cccc(Nc2ncccc2C(=O)OCC(O)CO)c1C]

Compound 35 was prepared using a procedure analogous to that of compound 19. Yield 35.0%. ¹H NMR (600 MHz, chloroform-d) δ 9.60 (br s, 1H), 8.11-8.27 (m, 2H), 7.51 (d, J=7.63 Hz, 1H), 7.29 (s, 1H), 7.19 (s, 1H), 7.06 (t, J=7.81 Hz, 1H), 6.94 (d, J=7.27 Hz, 1H), 6.58 (dd, J=7.99, 4.72 Hz, 1H), 4.24-4.52 (m, 2H), 4.01 (br s, 1H), 3.49-3.81 (m, 2H), 2.26 (s, 3H), 2.14 (s, 3H) ppm. ¹³C NMR (151 MHz, chloroform-d) δ 167.9, 157.3, 154.1, 140.3, 137.4, 130.6, 128.4, 126.7, 125.6, 122.6, 112.7, 106.0, 70.2, 65.8, 63.4, 20.8, 14.2 ppm.

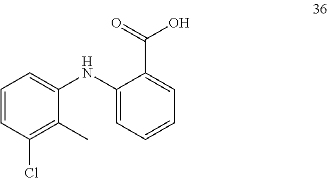 [SMILES: Cc1c(Cl)cccc1Nc1ccccc1C(=O)O]

Tolfenamic acid (36) was synthesized using the procedure disclosed in CN112624936A, which is incorporated by reference herein in its entirety.

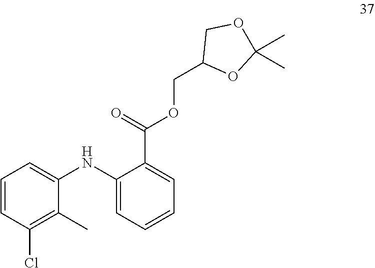 [SMILES: Cc1c(Cl)cccc1Nc1ccccc1C(=O)OCC1COC(C)(C)O1]

Compound 37 was prepared using a procedure analogous to that of compounds 9-13. Product was brought to next step without further purification.

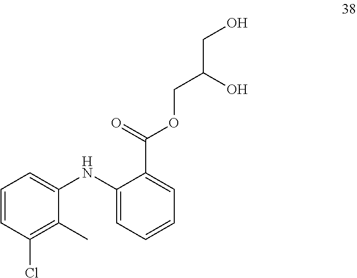 [SMILES: Cc1c(Cl)cccc1Nc1ccccc1C(=O)OCC(O)CO]

Compound 38 was prepared using a procedure analogous to that of compound 19. Yield 14.2%

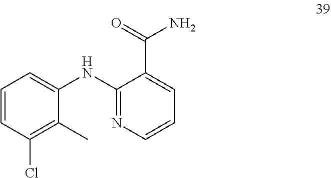 [SMILES: Cc1c(Cl)cccc1Nc1ncccc1C(N)=O]

To a round bottom flask, compound 8 (1 g, 3.81 mmol) was added with anhydrous DCM (7.5 ml) and charged with oxalyl chloride (0.400 mL, 4.57 mmol) at room temperature under argon. DMF was added dropwise, and the mixture was stirred for 2 hours. Once the reaction was completed, solvent was removed, and crude acid chloride was added slowly to an ice-cold solution of NH₄₀H. Acetonitrile was added in small amounts to aid in addition. Mixture was stirred for 20 minutes before product was extracted with ethyl acetate and purified using column chromatography (1:2 hexanes:ethyl acetate) to provide compound 39. Yield 0.304 g (30.5%).

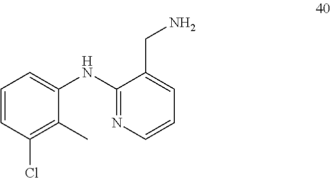 [SMILES: Cc1c(Cl)cccc1Nc1ncccc1CN]

In a round bottom flask, borane-methyl sulfide complex (0.057 ml, 0.602 mmol) was added dropwise under inert atmosphere at 0° C. to a stirred solution of compound 39 (0.150 g, 0.573 mmol) in toluene (6 ml). The reaction mixture was stirred at 0° C. for 15 minutes before being brought to reflux overnight. The next day, the flask was cooled to room temperature, 15 mL of saturated aqueous sodium carbonate was added, and the reaction mixture was stirred at 20° C. for 30 minutes. The toluene layer was separated and dried with sodium sulfate and the solvent was removed under vacuum to give 0.139 g of pure compound 40 (98%). ¹H NMR (600 MHz, chloroform-d) δ 2.29 (s, 3H), 3.90 (s, 2H), 6.55-6.65 (m, 1H), 6.92-7.09 (m, 2 H), 7.23-7.29 (m, 1H), 7.75-7.88 (m, 1H), 8.01-8.11 (m, 1H), 8.96-9.44 (m, 1H) ppm. ¹³C NMR (151 MHz, chloroform-d) δ 115.8, 146.7, 141.1, 136.8, 134.7, 126.6, 126.1, 122.7, 121.0, 119.0, 114.4, 45.3, 15.1 ppm.

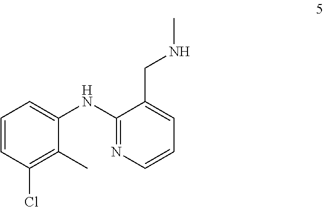 [SMILES: CNCc1cccnc1Nc1cccc(Cl)c1C]

Compound 5 was prepared using a procedure analogous to that of compound 40. ¹H NMR (600 MHz, chloroform-d) δ 8.14 (dd, J=5.09, 1.45 Hz, 1H), 7.77-7.93 (m, 1H), 7.35-7.43 (m, 1H), 7.07-7.14 (m, 1H), 7.02-7.14 (m, 2H), 6.65-6.76 (m, 1H), 3.87 (s, 2H), 2.50 (s, 3H), 2.32 (s, 3H), ppm. ¹³C NMR (151 MHz, chloroform-d) δ 154.67, 145.61, 140.02, 136.20, 133.59, 125.59, 124.57, 121.39, 118.30, 117.69, 113.29, 53.63, 34.41, 13.78 ppm.

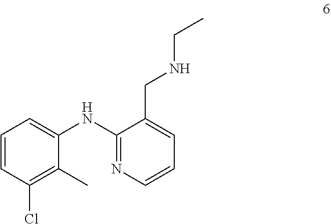 [SMILES: CCNCc1cccnc1Nc1cccc(Cl)c1C]

Compound 6 was prepared using a procedure analogous to that of compound 40. 1H NMR (600 MHz, chloroform-d) δ 9.16-9.63 (m, 1H), 8.15 (dd, J=4.72, 1.45 Hz, 1H), 7.96 (br d, J=7.99 Hz, 1H), 7.37 (br d, J=7.27 Hz, 1H), 7.10-7.17 (m, 1H), 7.01-7.08 (m, 1H), 6.65-6.73 (m, 1H), 3.88 (s, 2H), 2.75 (d, J=6.90 Hz, 2H), 2.39 (s, 3H), 1.21 (t, J=7.08 Hz, 3H) ppm. ¹³C NMR (151 MHz, chloroform-d) δ 155.7, 146.7, 141.1, 137.2, 134.7, 126.6, 125.7, 122.5, 119.5, 118.8, 114.4, 52.5, 43.4, 15.1 ppm.

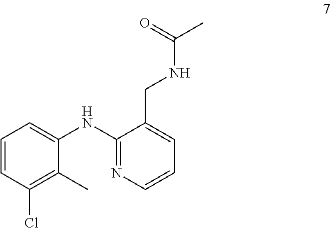 [SMILES: CC(=O)NCc1cccnc1Nc1cccc(Cl)c1C]

In a round bottom flask, compound 40 (0.070 g, 0.284 mmol) was dissolved in DCM (1.5 mL) and cooled to 0° C. After cooling, triethylamine (0.160 mL, 1.14 mmol) and acetic anhydride (0.080 mL, 0.853 mmol) were added to the solution with DMAP (3.47 mg, 0.028 mmol) added as catalyst. The mixture was stirred for 30 minutes at 0° C. before being brought to room temperature and allowed to stir overnight. The next day, water was added to the reaction and the organic layer was separated, washed with brine, and dried over sodium sulfate. The crude organic material was purified via column chromatography (1:1 hexanes:ethyl acetate) to yield 0.53 g of 7 (64.4%). ¹H NMR (600 MHz, methanol-d4) δ 7.85-7.90 (m, 1H), 7.50-7.54 (m, 1H), 7.17-7.21 (m, 2H), 7.09-7.13 (m, 1H), 6.72-6.75 (m, 1H), 4.38 (s, 2H), 2.24 (s, 3H), 1.99 (s, 3H) ppm. ¹³C NMR (151 MHz, methanol-d4) δ 172.64, 154.93, 146.06, 140.66, 138.55, 134.72, 131.27, 126.50, 125.09, 123.54, 119.72, 114.36, 39.44, 21.04, 14.27 ppm.

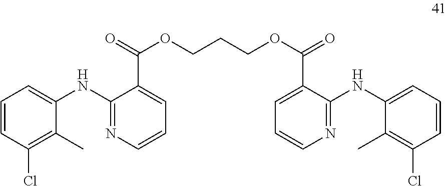 [SMILES: Cc1c(Cl)cccc1Nc1ncccc1C(=O)OCCCOC(=O)c1cccnc1Nc1cccc(Cl)c1C]

In a round bottom flask, 8 (0.444 g 1.69 mmol) in DCM (Volume: 8 mL) was brought to 0° C. A mixture of EDC (0.324 g, 1.69 mmol), DMAP (8.25 mg, 0.068 μmol), and propane-1,3-diol (0.048 mL, 0.675 mmol) was added to the reaction while stirring at 0° C. for 1 h before being brought to room temperature and stirred overnight. The reaction was quenched with water and organic layer was washed with saturated NaHCO₃ and brine. The resulting oil was purified via column chromatography (5% MeOH in DCM) to yield 41. HRMS m/z: [M+H]⁺ calc'd for C₂₉H₂₇Cl₂N₄O₄ 565.1409; Found 565.1408 ppm.

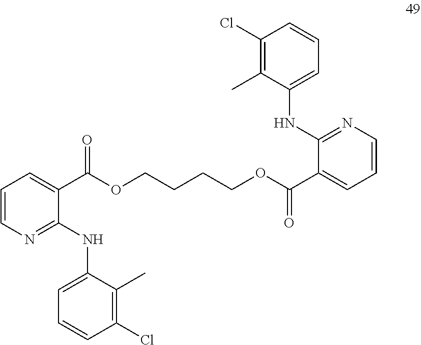 [SMILES: Cc1c(Cl)cccc1Nc1ncccc1C(=O)OCCCCOC(=O)c1cccnc1Nc1cccc(Cl)c1C]

Compound 49 was prepared using the analogous procedure to that of 41. HRMS m/z: [M+H]⁺ calc'd for C₃₀H₂₉Cl₂N₄O₄ 579.1566; Found 579.1563 ppm.

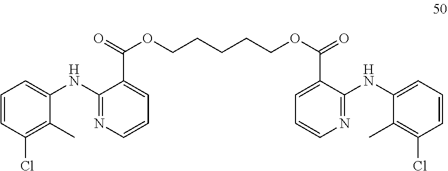 [SMILES: Cc1c(Cl)cccc1Nc1ncccc1C(=O)OCCCCCOC(=O)c1cccnc1Nc1cccc(Cl)c1C]

Compound 50 was prepared using the analogous procedure to that of 41. HRMS m/z: [M+H]⁺ calc'd for C₃₁H₃₁Cl₂N₄O₄ 593.1722; Found 593.1715 ppm.

### Example 2: Microscale Thermophoresis (MST) Analysis

### General Procedure 1

Nanotemper Monolith NT.115 labeled thermophoresis machine was used with standard treated capillary tubes using samples comprised of labeled protein and titrations of small molecule in 1×PBS. Microscale thermophoresis (MST) experiments were conducted in triplicate mixing 200 nM protein with 100 nM dye and allowing to sit at room temperature for 30 minutes followed by centrifugation on Ni-NTA 488 labeled His-labeled STING. Detection of the protein was performed using the blue detection channel with LED excitation power set to 90% and MST set to high allowing 3 s prior to MST on to check for initial fluorescence differences, 25 s for thermophoresis, and 3 s for regeneration after MST off. Analysis was performed using M.O. Affinity Analysis Software with difference between initial fluorescence measured in the first 5 s as compared with thermophoresis at 15 s at 15 different analyte concentrations ranging from 15 nM to 1 mM and exported into Graphpad Prism v.8 using a Log inhibitor v. response 4 parameter fit.

General Procedure 2 Nanotemper Monolith NT.115 labeled thermophoresis machine was used with standard treated capillary tubes using samples comprised of labeled protein and titrations of small molecule in 1× HBS-P. Microscale thermophoresis (MST) experiments were conducted in triplicate mixing 200 nM protein with 100 nM dye and allowing to sit at room temperature for 1 hour followed by centrifugation on Ni-NTA 488 labeled His-labeled STING. Detection of the protein was performed using the blue detection channel with LED excitation power set to 90% and MST set to high allowing 3 s prior to MST on to check for initial fluorescence differences, 25 s for thermophoresis, and 3 s for regeneration after MST off. Analysis was performed using M.O. Affinity Analysis Software with difference between initial fluorescence measured in the first 5 s as compared with thermophoresis at 15 s at 15 different analyte concentrations ranging from 100 zM to 100 μM and exported into Graphpad Prism v.8 using a Log inhibitor v. response 4 parameter fit.

### Results

Microscale Thermophoresis (MST) was performed on compound 1 incubating with 50 nM 2,3-cGAMP. Confidence is categorized as follows: 1=low confidence (<2 MST shift unit−non-statistical); 2=mid confidence (2-3 MST shift units−intermediate); 3=high confidence (>2 MST shift units−statistical). Dissociation constants are categorized as follows: A<1 μM; 1 μM≤B<100 μM; 100 μM≤C<10 μM; 10 μM≤D<100 μM. The results are shown in Table A below:

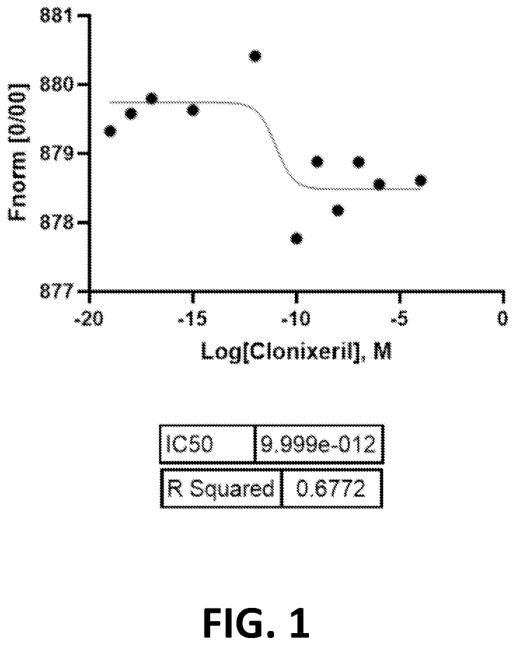

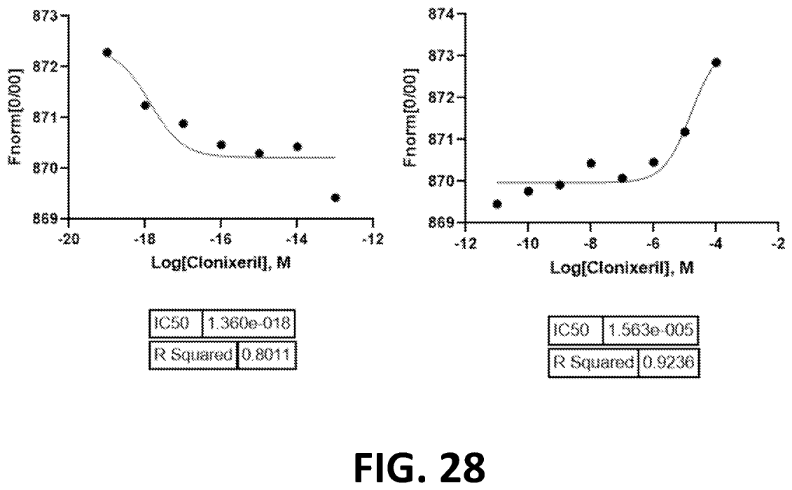

TABLE A
Dissociation Constant of Compounds 1 and 40.

| Compound | Dose-Response Curve | Dissociation Constant | Confidence |
|---|---|---|---|
| 1 | FIG. 1 | B | 1 |
| 40 | FIG. 28 | K_{d1}: D; K_{d2}: A | Not available |

Reference compounds were assayed using the same protocol. The structures and dissociation constants of the reference compounds are shown in Table B below.

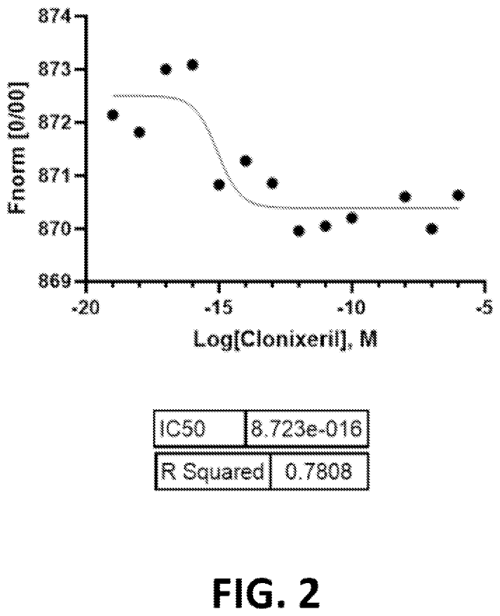

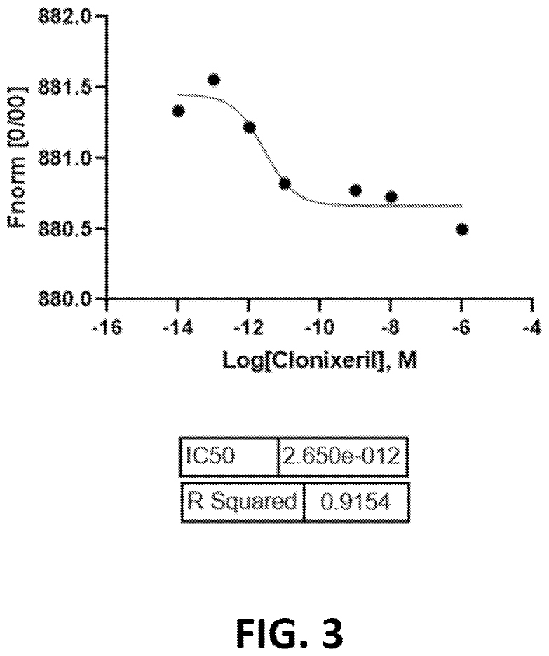

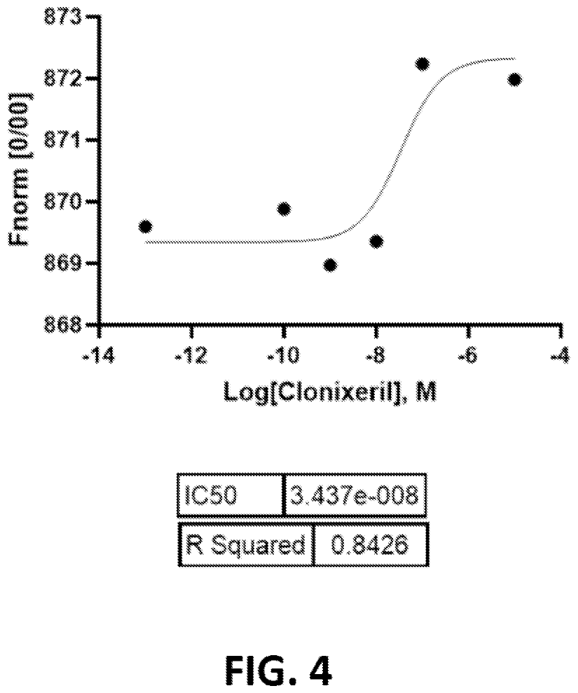

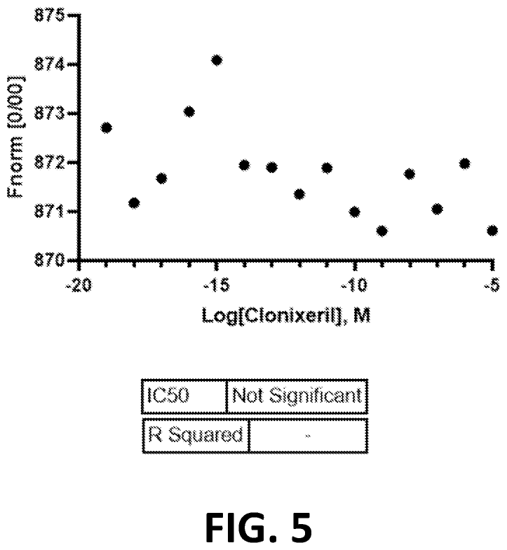

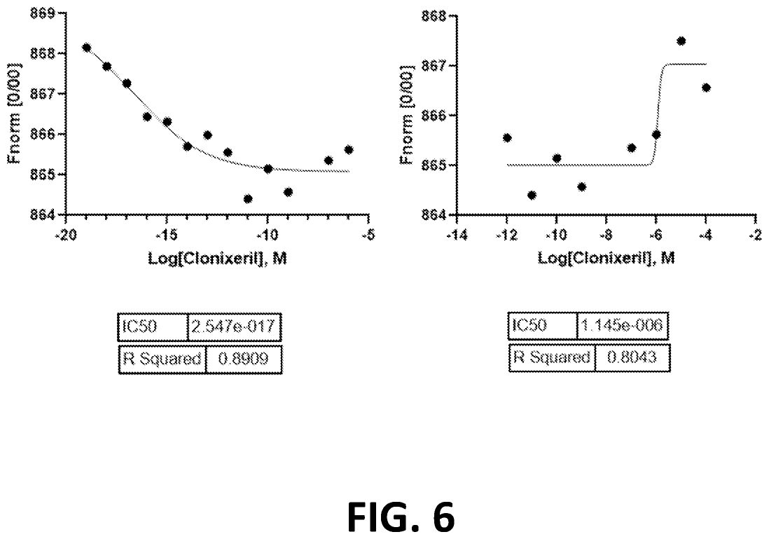

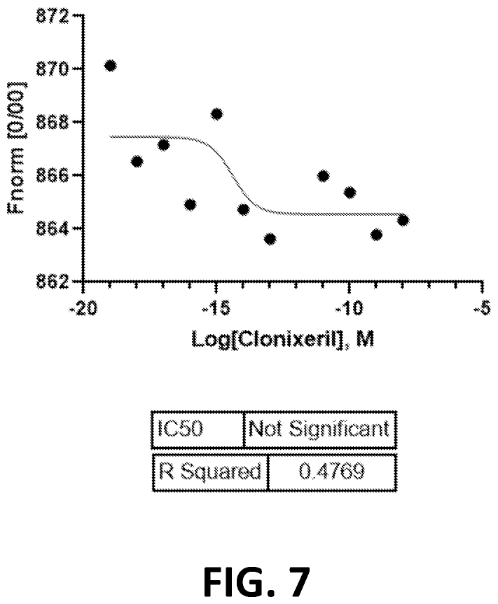

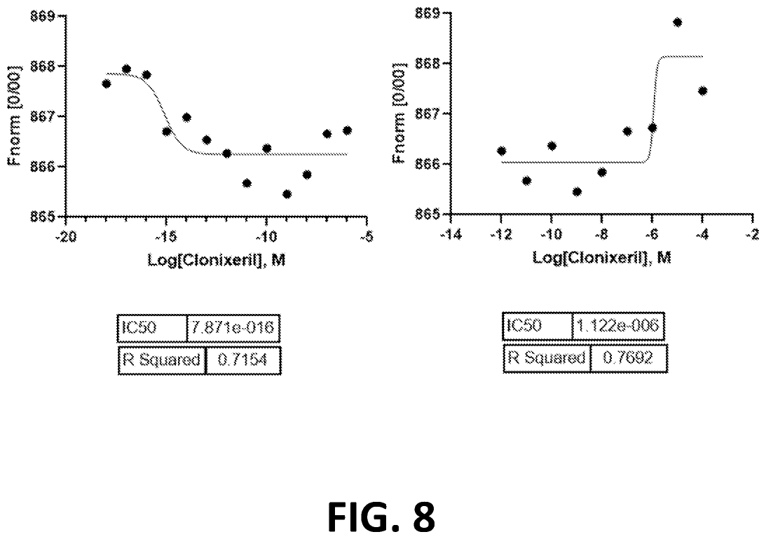

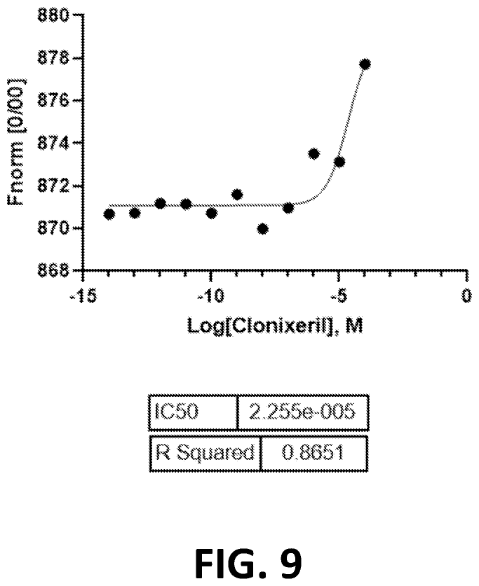

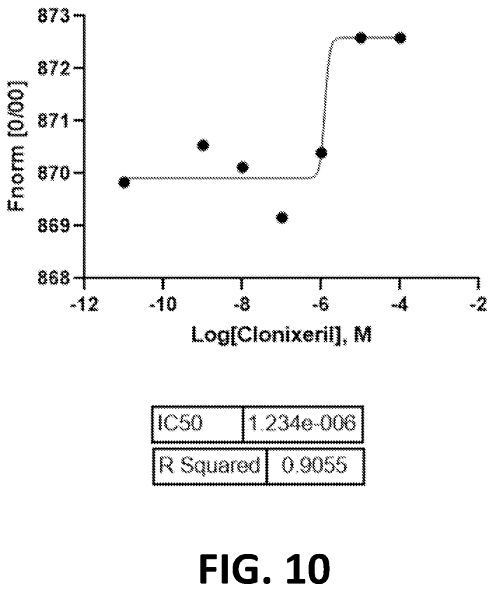

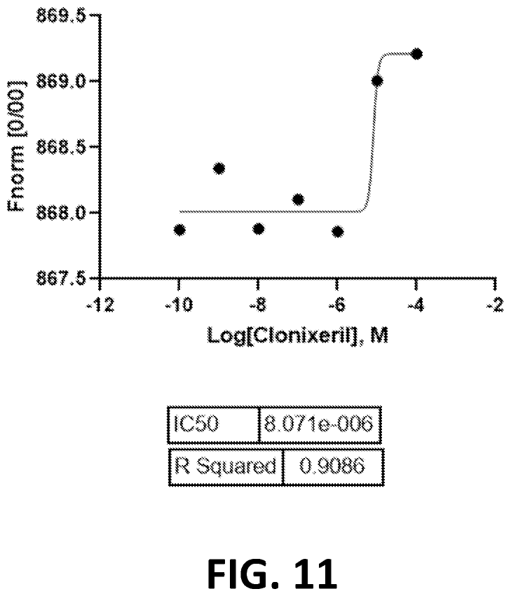

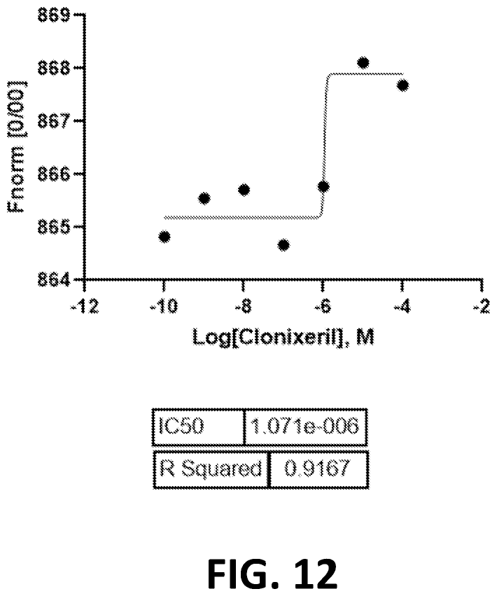

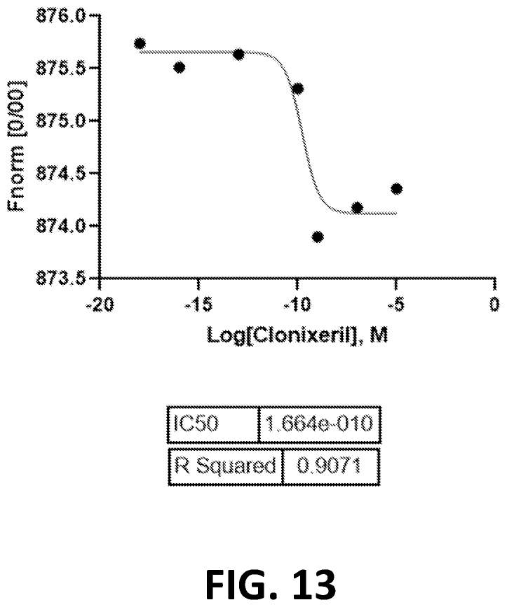

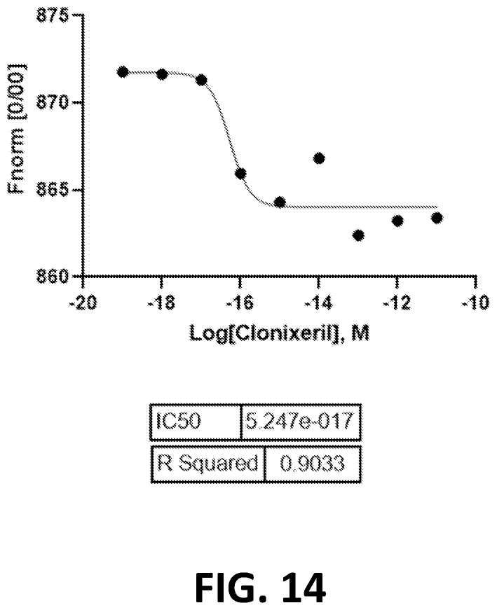

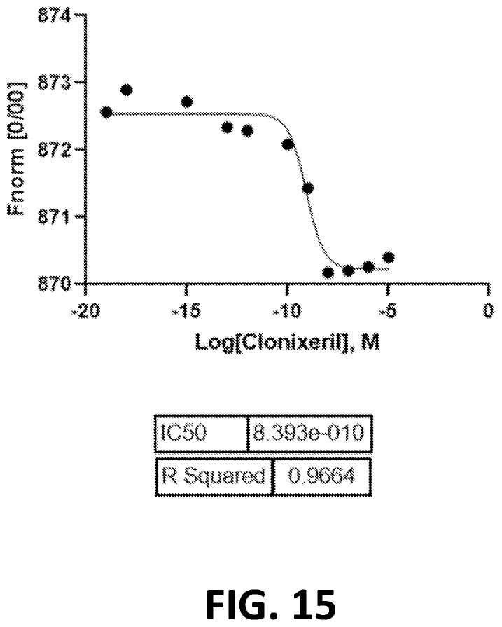

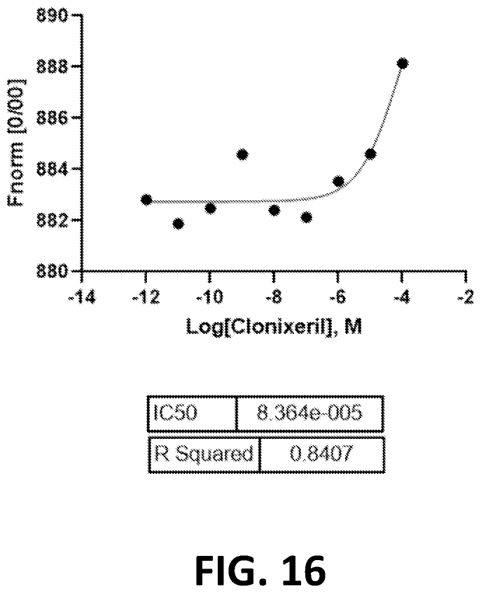

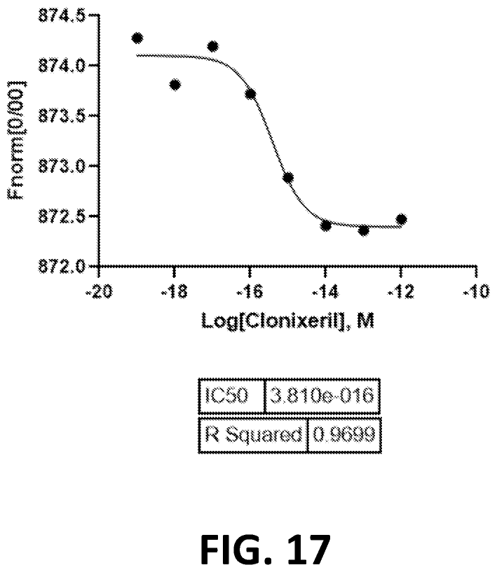

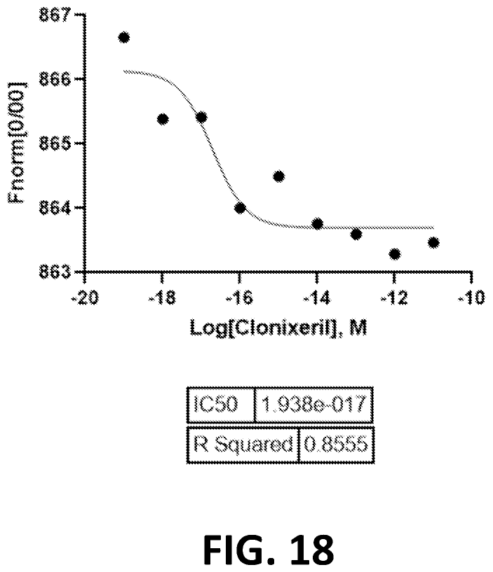

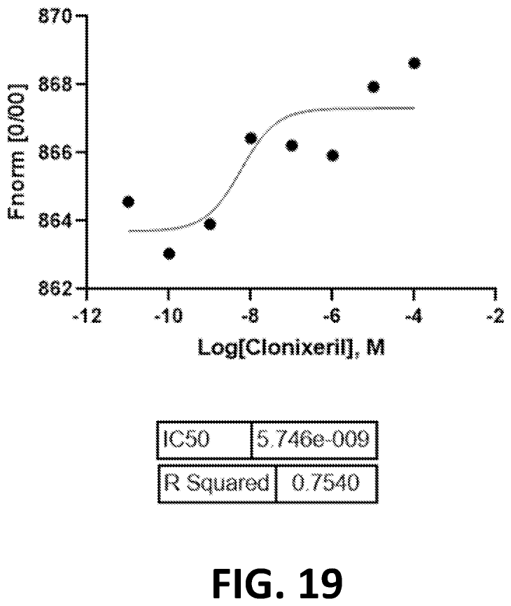

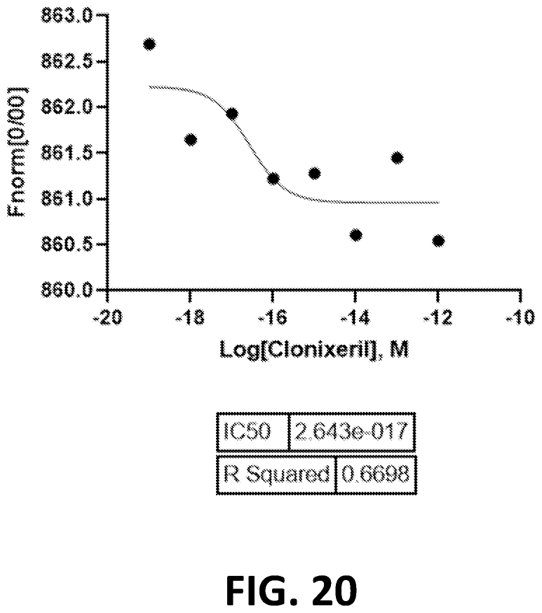

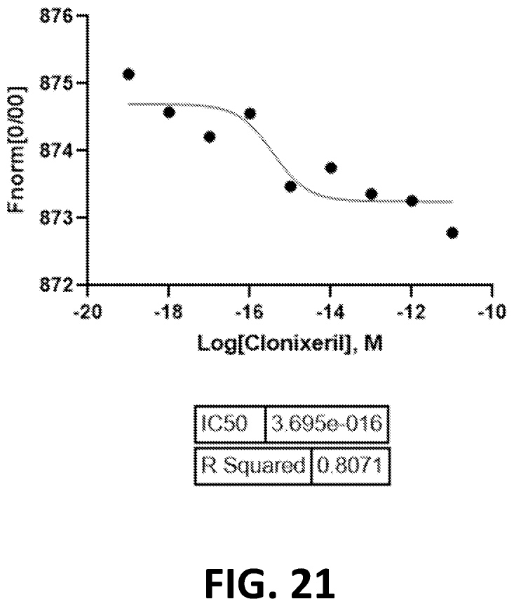

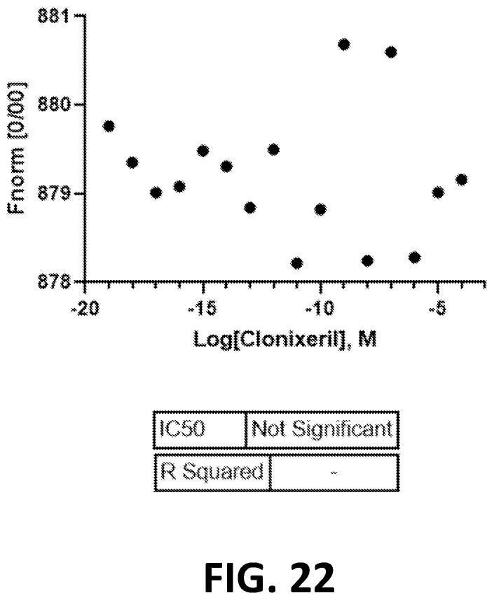

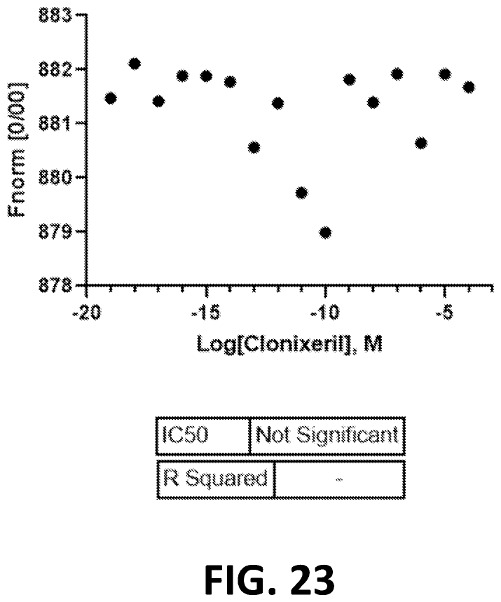

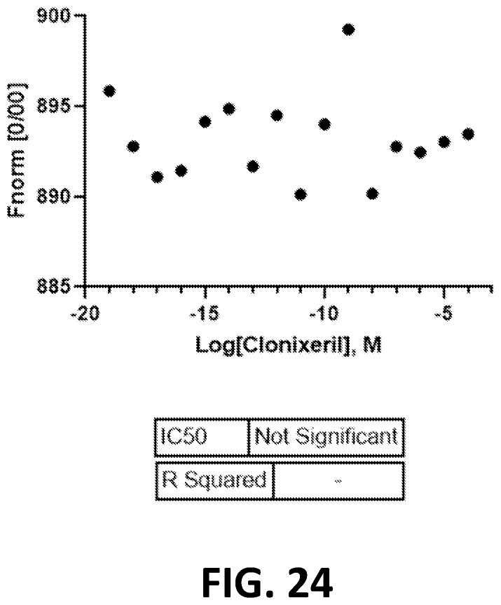

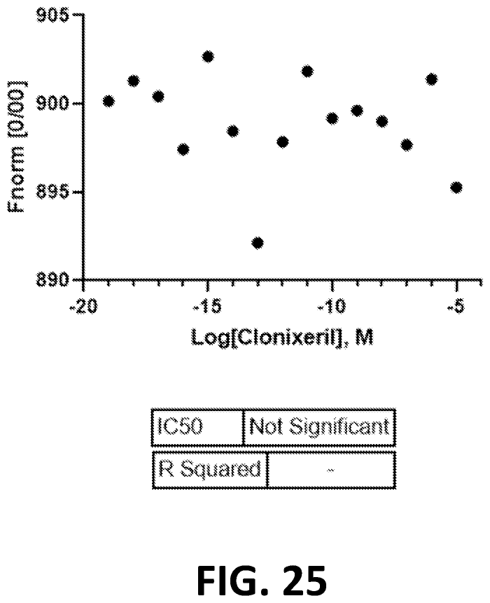

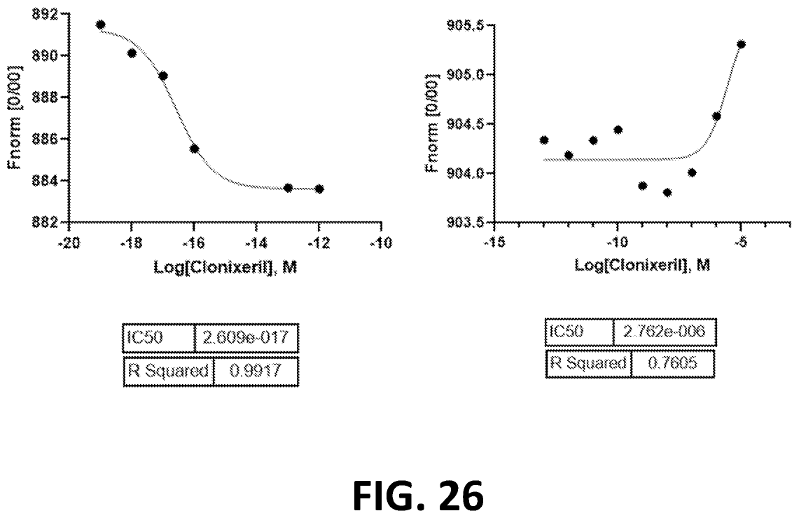

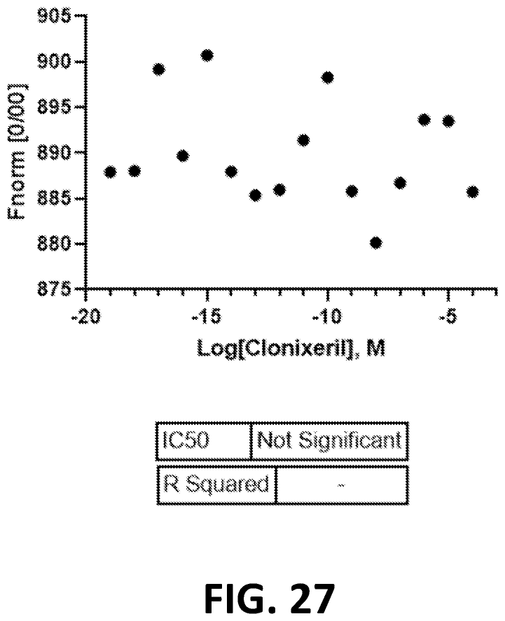

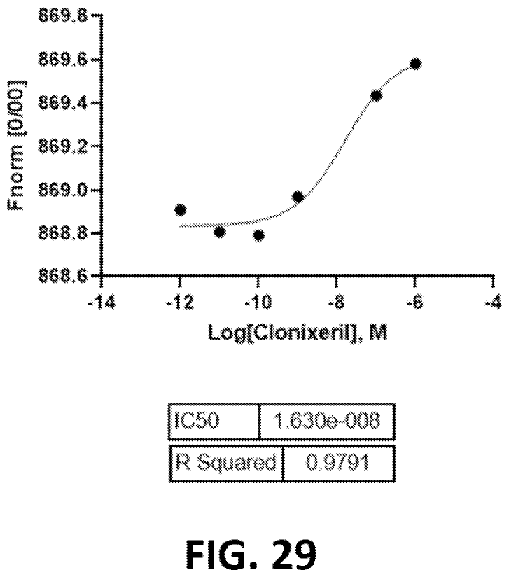

TABLE B
Dissociation Constants and Dose-Response Curves of Reference Compounds.

| | | Dose- | | |
|---|---|---|---|---|
| | | Response | Dissociation | |
| Compound | Structure | Curve | Constant | Confidence |
| 4 | 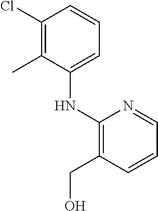 [SMILES: Cc1c(Cl)cccc1Nc1ncccc1CO] | FIG. 2 | A | 2 |
| 8 | 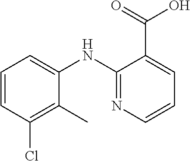 [SMILES: Cc1c(Cl)cccc1Nc1ncccc1C(=O)O] | FIG. 5 | No Binding | 1 |
| 9 | 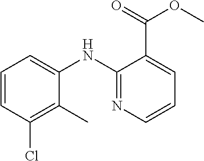 [SMILES: COC(=O)c1cccnc1Nc1cccc(Cl)c1C] | FIG. 6 | K_{d1}: C; K_{d2}: A | 3 |
| 10 | 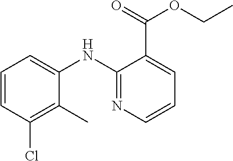 [SMILES: CCOC(=O)c1cccnc1Nc1cccc(Cl)c1C] | FIG. 7 | Low R² | 1 |
| 11 | 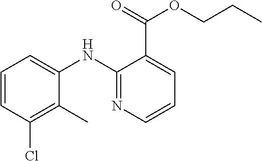 [SMILES: CCCOC(=O)c1cccnc1Nc1cccc(Cl)c1C] | FIG. 8 | K_{d1}: C; K_{d2}: A | 1 |
| 13 | 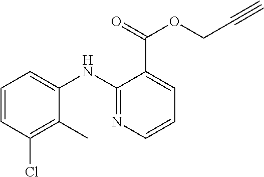 [SMILES: C#CCOC(=O)c1cccnc1Nc1cccc(Cl)c1C] | FIG. 9 | D | 2 |
| 14 | 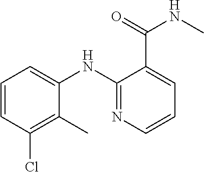 [SMILES: CNC(=O)c1cccnc1Nc1cccc(Cl)c1C] | FIG. 10 | C | 2 |
| 15 | 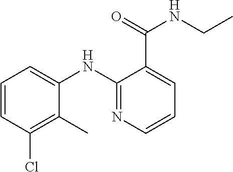 [SMILES: CCNC(=O)c1cccnc1Nc1cccc(Cl)c1C] | FIG. 11 | C | 1 |
| 16 | 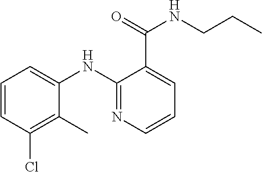 [SMILES: CCCNC(=O)c1cccnc1Nc1cccc(Cl)c1C] | FIG. 12 | C | 2 |
| 17 | 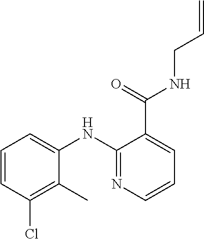 [SMILES: C=CCNC(=O)c1cccnc1Nc1cccc(Cl)c1C] | FIG. 13 | C | 1 |
| 19 | 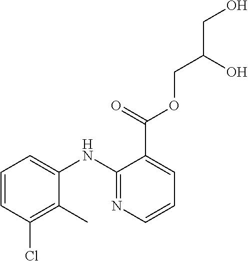 [SMILES: Cc1c(Cl)cccc1Nc1ncccc1C(=O)OCC(O)CO] | FIG. 14 | A | 1 |
| 21 | 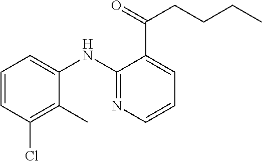 [SMILES: CCCCC(=O)c1cccnc1Nc1cccc(Cl)c1C] | FIG. 15 | C | 2 |
| 23 | 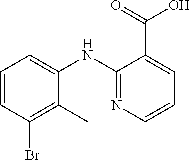 [SMILES: Cc1c(Br)cccc1Nc1ncccc1C(=O)O] | FIG. 16 | D | 3 |
| 24 | 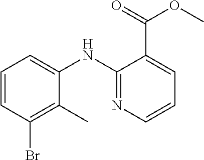 [SMILES: COC(=O)c1cccnc1Nc1cccc(Br)c1C] | FIG. 17 | B | 1 |
| 26 | 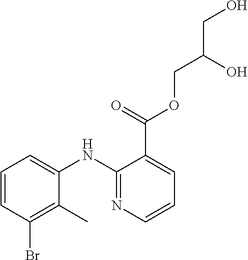 [SMILES: Cc1c(Br)cccc1Nc1ncccc1C(=O)OCC(O)CO] | FIG. 18 | A | Not available |
| 27 | 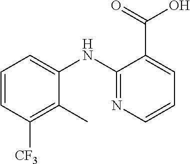 [SMILES: Cc1c(Nc2ncccc2C(=O)O)cccc1C(F)(F)F] | FIG. 19 | C | 3 |
| 29 | 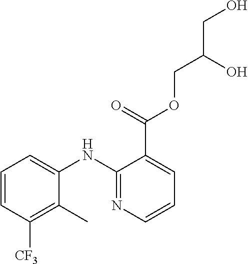 [SMILES: Cc1c(Nc2ncccc2C(=O)OCC(O)CO)cccc1C(F)(F)F] | FIG. 20 | A | Not available |
| 30 | 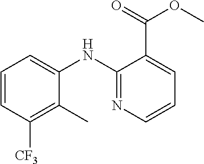 [SMILES: COC(=O)c1cccnc1Nc1cccc(C(F)(F)F)c1C] | FIG. 21 | B | Not available |
| 32 | 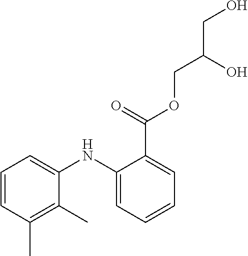 [SMILES: Cc1cccc(Nc2ccccc2C(=O)OCC(O)CO)c1C] | FIG. 22 | Not available | Not available |
| 33 | 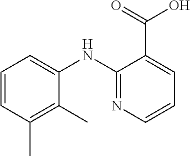 [SMILES: Cc1cccc(Nc2ncccc2C(=O)O)c1C] | FIG. 23 | Not available | Not available |
| 35 | 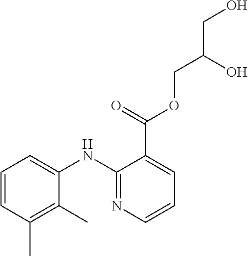 [SMILES: Cc1cccc(Nc2ncccc2C(=O)OCC(O)CO)c1C] | FIG. 24 | Not available | Not available |
| 36 | 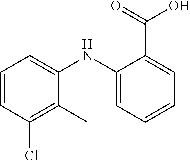 [SMILES: Cc1c(Cl)cccc1Nc1ccccc1C(=O)O] | FIG. 25 | No Binding | 1 |
| 38 | 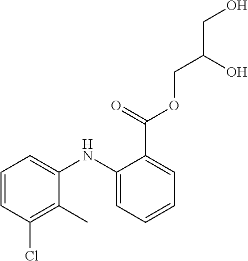 [SMILES: Cc1c(Cl)cccc1Nc1ccccc1C(=O)OCC(O)CO] | FIG. 26 | K_{d1}: C; K_{d2}: A | 1 |
| 39 | 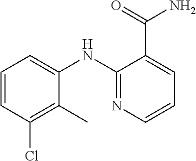 [SMILES: Cc1c(Cl)cccc1Nc1ncccc1C(N)=O] | FIG. 27 | Not available | Not available |
| 41 | 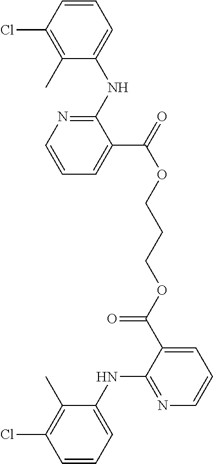 [SMILES: Cc1c(Cl)cccc1Nc1ncccc1C(=O)OCCCOC(=O)c1cccnc1Nc1cccc(Cl)c1C] | FIG. 29 | C | 1 |
| 42 | 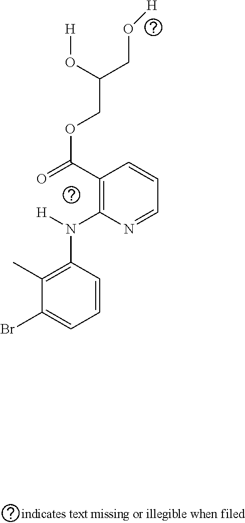 [SMILES: Cc1c(Br)cccc1Nc1ncccc1C(=O)OCC(O)CO] | Not available | A | 2 |
| 43 | 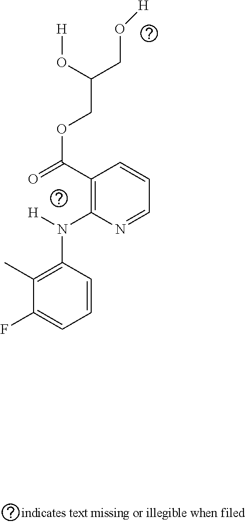 [SMILES: Cc1c(F)cccc1Nc1ncccc1C(=O)OCC(O)CO] | Not available | A | 1 |
| 44 | 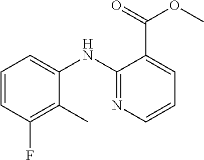 [SMILES: COC(=O)c1cccnc1Nc1cccc(F)c1C] | Not available | B | 1 |
| 45 | 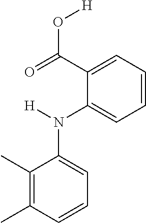 [SMILES: Cc1cccc(Nc2ccccc2C(=O)O)c1C] | Not available | No Binding | 1 |
| 46 | 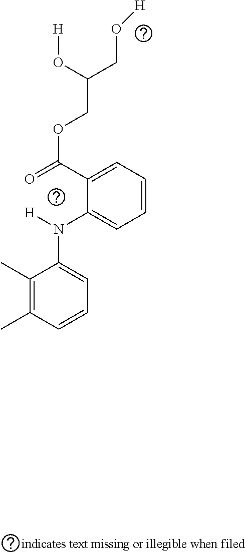 [SMILES: Cc1cccc(Nc2ccccc2C(=O)OCC(O)CO)c1C] | Not available | No Binding | 1 |
| 47 | 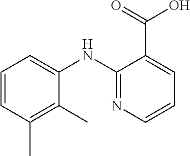 [SMILES: Cc1cccc(Nc2ncccc2C(=O)O)c1C] | Not available | No Binding | 1 |
| 48 | 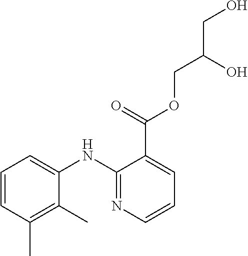 [SMILES: Cc1cccc(Nc2ncccc2C(=O)OCC(O)CO)c1C] | Not available | No Binding | 1 |
| 49 | 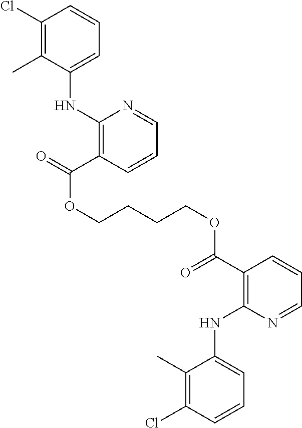 [SMILES: Cc1c(Cl)cccc1Nc1ncccc1C(=O)OCCCCOC(=O)c1cccnc1Nc1cccc(Cl)c1C] | FIG. 3 | B | 1 |
| 50 | 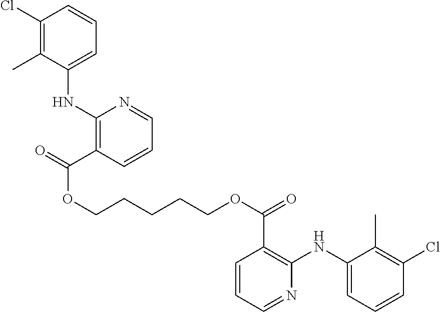 [SMILES: Cc1c(Cl)cccc1Nc1ncccc1C(=O)OCCCCCOC(=O)c1cccnc1Nc1cccc(Cl)c1C] | FIG. 4 | C | 3 |
| 52 | 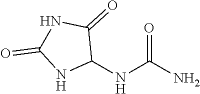 [SMILES: NC(=O)NC1NC(=O)NC1=O] | Not available | K_{d1}: C; K_{d2}: A | 3 |

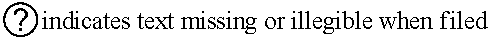

Although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the invention.

### REFERENCES

The following references are incorporated herein in their entirety:
Yi G. et al., 2013. PLoS One. 8:e77846.
Jin L. et al., 2011. Genes Immun. 12:263-9.
Zhang X. et al., 2013. Mol. Cell. 51:226-235.
Gao, Pu, et al. Cell 154.4 (2013): 748-762.
Chin, K-H., et al. Acta Crystallographica Section D: Biological Crystallography 69.3 (2013): 352-366.
Shu, Chang, et al. Nature structural and molecular biology 19.7 (2012): 722.
Shang, Guijun, et al. Nature structural and molecular biology 19.7 (2012): 725.
Sastry, G. M.; Adzhigirey, M.; Day, T.; Annabhimoju, R.; Sherman, W., J. Comput. Aid. Mol. Des., 2013, 27(3), 221-234.
Li, Hui, Andrew D. Robertson, and Jan H. Jensen. Proteins: Structure, Function, and Bioinformatics 61.4 (2005): 704-721.
Shivakumar, D.; Williams, J.; Wu, Y; Damm, W.; Shelley, J.; Sherman, W., J. Chem. Theory Comput., 2010, 6, 1509-1519
Guo, Z.; Mohanty, U.; Noehre, J.; Sawyer, T. K.; Sherman, W.; Krilov, G., Chem. Biol. Drug Des., 2010, 75, 348-359
Kevin J. Bowers, Edmond Chow, Huafeng Xu, Ron O. Dror, Michael P. Eastwood, Brent A. Gregersen, John L. Klepeis, Istvan Kolossvary, Mark A. Moraes, Federico D. Sacerdoti, John K. Salmon, Yibing Shan, and David E. Shaw, Proceedings of the ACM/IEEE Conference on Supercomputing (SC06), Tampa, Florida, 2006, November 11-17.
Price, Daniel J., and Charles L. Brooks III. The Journal of chemical physics 121.20 (2004): 10096-10103.
Harder, E.; Damm, W.; Maple, J.; Wu, C.; Reboul, M.; Xiang, J. Y.; Wang, L.; Lupyan, D.; Dahlgren, M. K.; Knight, J. L.; Kaus, J. W.; Cerutti, D.S.; Krilov, G.; Jorgensen, W. L.; Abel, R.; Friesner, R. A., J. Chem. Theory Comput., 2015, DOI: 10.1021/acs.jctc.5b00864]
Ryckaert, Jean-Paul, Giovanni Ciccotti, and Herman J C Berendsen. Journal of Computational Physics 23.3 (1977): 327-341.
Tuckerman, M. B. B. J. M., Bruce J. Berne, and Glenn J. Martyna. The Journal of chemical physics 97.3 (1992): 1990-2001.
Darden, T.; York, D.; Pedersen, L. Particle mesh ewald—an N. Log(N) method for Ewald sums in large systems. J. Chem. Phys. 1993, 98, 10089-10092
Essmann, U.; Perera, L.; Berkowitz, M. L.; Darden, T.; Lee, H.; Pedersen, L. G. A smooth particle mesh Ewald method. J. Chem. Phys. 1995, 103, 8577-8593
Nosé, Shuichi, and M. L. Klein. Molecular Physics 50.5 (1983): 1055-1076
Martyna, Glenn J., Douglas J. Tobias, and Michael L. Klein. The Journal of Chemical Physics 101.5 (1994): 4177-4189 && Martyna, Glenn J., et al. Molecular Physics 87.5 (1996): 1117-1157.
Ponder, Jay W., and Frederic M. Richards. Journal of Computational Chemistry 8.7 (1987): 1016-1024
Berendsen, H. J. C.; Postma, J. P. M.; van Gunsteren, W. F.; DiNola, A.; Haak, J. R. (1984). Journal of Chemical Physics 81 (8): 3684-3690.
Halgren, T., J. Chem. Inf. Model., 2009, 49, 377-389
Halgren, T., Chem. Biol. Drug Des., 2007, 69, 146-148.
Friesner, R. A.; Murphy, R. B.; Repasky, M. P.; Frye, L. L.; Greenwood, J. R.; Halgren, T. A.; Sanschagrin, P. C.; Mainz, D. T., J. Med Chem., 2006, 49, 6177-6196
Halgren, T. A.; Murphy, R. B.; Friesner, R. A.; Beard, H. S.; Frye, L. L.; Pollard, W. T.; Banks,
J. L., J. Med Chem., 2004, 47, 1750-1759
Friesner, R. A.; Banks, J. L.; Murphy, R. B.; Halgren, T. A.; Klicic, J. J.; Mainz, D. T.; Repasky, M. P.; Knoll, E. H.; Shaw, D. E.; Shelley, M.; Perry, J. K.; Francis, P.; Shenkin, P. S., J. Med Chem., 2004, 47, 1739-1749.
Farid, R.; Day, T.; Friesner, R. A.; Pearlstein, R. A., Bioorg. & Med Chem., 2006, 14, 3160-3173
Sherman, W.; Day, T.; Jacobson, M. P.; Friesner, R. A.; Farid, R., J. Med Chem., 2006, 49, 534-553
Sherman, W.; Beard, H. S.; Farid, R., Chemical Biology & Drug Design, 2006, 67, 83-84.
Cho, A. E.; Guallar, V.; Berne, B.; Friesner, R. A., J. Comput. Chem., 2005, 26, 915-931.
Murphy, R. B.; Philipp, D. M.; Friesner, R. A., J. Comp. Chem., 2000, 21, 1442-1457
Philipp, D. M.; Friesner, R. A., J. Comp. Chem., 1999, 20, 1468-1494.
Qiu, Di, et al. The Journal of Physical Chemistry A 101.16 (1997): 3005-3014.
Conlon, Joseph, et al., The Journal of Immunology 190.10 (2013): 5216-5225.
Koes, David Ryan, and Carlos J. Camacho. Nucleic acids research 40. W1 (2012): W409-W414.
Tanaka, Yasuo, and Zhijian J. Chen. Science signaling 5.214 (2012): ra20-ra20.
Gao, D.; Li, T.; Li, X.-D.; Chen, X.; Li, Q.-Z.; Wight-Carter, M.; Chen, Z. Proceedings of the National Academy of Sciences of the United States of America 2015, E5699-E5705.
Zhou, R.; Xie, X.; Li, X.; Qin, Z.; Wei, C.; Liu, J.; Luo, Y Infection, Genetics, and Evolution 2020, 77, 104094.
Ahn, J.; Barber, G. Current Opinion in Immunology 2014, 31, 121-126.
Li, Q.-Z.; Zhou, J.; Lian, Y; Zhang, B.; Branch, V. K.; Carr-Johnson, F.; Karp, D. R.; Mohan, C.; Wakeland, E. K.; Olsen, N. J. Clin. Exp. Immunol. 2010, 159(3), 281-291.

What is claimed is:

1. A compound of Formula (I), or a pharmaceutically acceptable salt thereof: 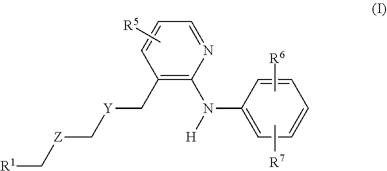
wherein:
Y and Y¹ are independently selected from O, NR^{4A}, CH₂, or absent;
Z is independently selected from C1-C10 alkylene, C2-C10 alkenylene, C2-C10 alkynylene, or 3-10 membered heteroalkylene, each optionally substituted with 1-3 independently selected R²;
R¹ and each occurrence of R² are independently selected from hydrogen, OH, OR³, Y¹R^{3A}, SR³, and NR³R⁴;
R³, R⁴, and R^{4A} are independently selected from hydrogen, C1-C10 alkyl optionally substituted with 1-6 halogens, C6-C10 aryl, and 5 to 10 membered heteroaryl; or
R³ and R⁴, together with the nitrogen atom to which they are attached, can come together to form a 3 to 7 membered heterocyclyl or 5 to 10 membered heteroaryl;
R^{3A} is 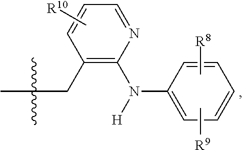
wherein [symbol] represents the point of connection of R^{3A} to the remainder of the molecule;
R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl; and
each occurrence of R′ is independently selected from H and C1-C6 alkyl.

2. The compound of claim 1, wherein R¹ and each occurrence of R² collectively comprise 0-1 occurrences of Y¹R^{3A}.

3. The compound of claim 1, with the proviso that when one occurrence of R¹ and R² is Y¹R^{3A}, the other occurrences of R¹ and R² are not Y¹R^{3A}.

4. The compound of any one of claims 1-3, wherein Y and Y¹ are independently selected from O and NR^{4A}.

5. The compound of any one of claims 1-4, wherein Y is absent.

6. The compound of any one of claims 1-4, wherein Y is O.

7. The compound of any one of claims 1-4, wherein Y is NR^{4A}.

8. The compound of claim 1 or 7, wherein R^{4A} is hydrogen.

9. The compound of claim 1 or 7, wherein R^{4A} is C1-C10 alkyl optionally substituted with 1-6 halogens.

10. The compound of claim 9, wherein R^{4A} is methyl.

11. The compound of claim 9, wherein R^{4A} is trifluoromethyl.

12. The compound of claim 1 or 7, wherein R^{4A} is C6-C10 aryl.

13. The compound of claim 1 or 7, wherein R^{4A} is 5 to 10 membered heteroaryl.

14. The compound of any one of claims 1-4, wherein Y is CH₂.

15. The compound of any one of claims 1-14, wherein Y¹ is absent.

16. The compound of any one of claims 1-14, wherein Y¹ is O.

17. The compound of any one of claims 1-14, wherein Y¹ is NR^{4A}.

18. The compound of claim 1 or 17, wherein R^{4A} is hydrogen.

19. The compound of claim 1 or 17, wherein R^{4A} is C1-C10 alkyl optionally substituted with 1-6 halogens.

20. The compound of claim 19, wherein R^{4A} is methyl.

21. The compound of claim 19, wherein R^{4A} is trifluoromethyl.

22. The compound of claim 1 or 17, wherein R^{4A} is C6-C10 aryl.

23. The compound of claim 1 or 17, wherein R^{4A} is 5 to 10 membered heteroaryl.

24. The compound of any one of claims 1-14, wherein Y¹ is CH₂.

25. The compound of any one of claims 1-24, wherein Z is C1-C10 alkylene optionally substituted with 1-3 independently selected R².

26. The compound of any one of claims 1-25, wherein Z is C1-C10 alkylene substituted with 1-3 independently selected R².

27. The compound of any one of claims 1-25, wherein Z is unsubstituted C1-C10 alkylene.

28. The compound of any one of claims 1-24, wherein Z is C2-C10 alkenylene optionally substituted with 1-3 independently selected R².

29. The compound of any one of claims 1-24 and 28, wherein Z is C2-C10 alkenylene substituted with 1-3 independently selected R².

30. The compound of any one of claims 1-24 and 28, wherein Z is unsubstituted C2-C10 alkenylene.

31. The compound of any one of claims 1-24, wherein Z is C2-C10 alkynylene optionally substituted with 1-3 independently selected R².

32. The compound of any one of claims 1-24 and 31, wherein Z is C2-C10 alkynylene substituted with 1-3 independently selected R².

33. The compound of any one of claims 1-24 and 31, wherein Z is unsubstituted C2-C10 alkynylene.

34. The compound of any one of claims 1-24, wherein Z is 3-10 membered heteroalkylene optionally substituted with 1-3 independently selected R².

35. The compound of any one of claims 1-24 and 34, wherein Z is 3-10 membered heteroalkylene substituted with 1-3 independently selected R².

36. The compound of any one of claims 1-24 and 34, wherein Z is unsubstituted 3-10 membered heteroalkylene.

37. The compound of any one of claims 1-36, wherein R¹ is hydrogen.

38. The compound of any one of claims 1-36, wherein R¹ is OH.

39. The compound of any one of claims 1-36, wherein R¹ is OR³.

40. The compound of any one of claims 1-36, wherein R¹ is Y¹R^{3A}.

41. The compound of any one of claims 1-36 and 40, wherein R¹ is R^{3A}.

42. The compound of any one of claims 1-36, wherein R¹ is SR³.

43. The compound of any one of claims 1-36, wherein R¹ is NR³R⁴.

44. The compound of any one of claims 1-36, wherein R¹ is selected from OH, OR³, Y¹R^{3A}, SR³, and NR³R⁴.

45. The compound of any one of claims 1-26, 28-29, 31-32, 34-35, and 37-44, wherein at least one R² is hydrogen.

46. The compound of any one of claims 1-26, 28-29, 31-32, 34-35, 37-39, and 42-44, wherein at least one R² is OH.

47. The compound of any one of claims 1-26, 28-29, 31-32, 34-35, 37-39, and 42-44, wherein at least one R² is OR³.

48. The compound of any one of claims 1-26, 28-29, 31-32, 34-35, 37-39, and 42-44, wherein at least one R² is Y¹R^{3A}.

49. The compound of any one of claims 1-26, 28-29, 31-32, 34-35, 37-39, 42-44, and 48, wherein at least one R² is R^{3A}.

50. The compound of any one of claims 1-26, 28-29, 31-32, 34-35, 37-39, and 42-44, wherein at least one R² is SR³.

51. The compound of any one of claims 1-26, 28-29, 31-32, 34-35, 37-39, and 42-44, wherein at least one R² is NR³R⁴.

52. The compound of any one of claims 1-26, 28-29, 31-32, 34-35, 37-39, and 42-44, wherein at least one R² is selected from OH, OR³, Y¹R^{3A}, SR³, and NR³R⁴.

53. The compound of any one of claims 1-26, 28-29, 31-32, and 34-35, wherein R¹ and each occurrence of R² are independently selected from OH, OR³, and Y¹R^{3A}.

54. The compound of any one of claims 1-26, 28-29, 31-32, and 34-35, wherein R¹ is Y¹R^{3A} and each occurrence of R² is selected from OH and OR³.

55. The compound of any one of claims 1-26, 28-29, 31-32, and 34-35, wherein R¹ and each occurrence of R² are each OH.

56. The compound of any one of claims 1-36, 39, and 42-54, wherein R³ is hydrogen.

57. The compound of any one of claims 1-36, 39, and 42-54, wherein R³ is C1-C10 alkyl optionally substituted with 1-6 halogens.

58. The compound of claim 57, wherein R³ is methyl.

59. The compound of claim 57, wherein R³ is trifluoromethyl.

60. The compound of any one of claims 1-36, 39, and 42-54, wherein R³ is C6-C10 aryl.

61. The compound of any one of claims 1-36, 39, and 42-54, wherein R³ is 5 to 10 membered heteroaryl.

62. The compound of any one of claims 1-36, 43-44, 51-52, and 56-61, wherein R⁴ is hydrogen.

63. The compound of any one of claims 1-36, 43-44, 51-52, and 56-61, wherein R⁴ is C1-C10 alkyl optionally substituted with 1-6 halogens.

64. The compound of claim 63, wherein R⁴ is methyl.

65. The compound of claim 63, wherein R⁴ is trifluoromethyl.

66. The compound of any one of claims 1-36, 43-44, 51-52, and 56-61, wherein R⁴ is C6-C10 aryl.

67. The compound of any one of claims 1-36, 43-44, 51-52, and 56-61, wherein R⁴ is 5 to 10 membered heteroaryl.

68. The compound of any one of claims 1-36, 43-44, 51-52, and 56-61, wherein R³ and R⁴, together with the nitrogen atom to which they are attached, come together to form a 3 to 7 membered heterocyclyl.

69. The compound of any one of claims 1-36, 43-44, 51-52, and 56-61, wherein R³ and R⁴, together with the nitrogen atom to which they are attached, come together to form a 5 to 10 membered heteroaryl.

70. A compound of Formula (II), or a pharmaceutically acceptable salt thereof: 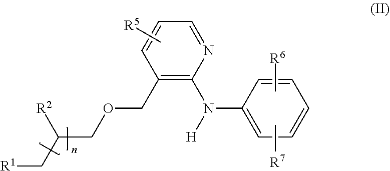
wherein:
R¹ is selected from OH, OR, SR³, NR³R⁴, and 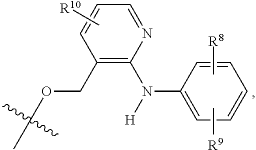
wherein [symbol] represents the point of connection of R¹ to the remainder of the molecule;
n is 0, 1, 2, or 3;
each occurrence of R² is an independently selected hydrogen, OH, OR³, SR³, or NR³R⁴;
R³ and R⁴ are independently selected from hydrogen, C1-C10 alkyl optionally substituted with 1-6 halogens, C6-C10 aryl, and 5 to 10 membered heteroaryl; or
R³ and R⁴, together with the nitrogen atom to which they are attached, can come together to form a 3 to 7 membered heterocyclyl or 5 to 10 membered heteroaryl; and
R⁵, R⁶, R⁷, R⁸, R⁹, and R¹⁰ are independently selected from hydrogen, halogen, pseudohalogen, C1-C10 alkyl optionally substituted with 1-6 halogens, C1-C6 alkoxy, cyano, nitro, C(O)C1-C6 alkyl, CO₂C1-C6 alkyl, C(O)NHC1-C6 alkyl, N(R′)₂, C6-C10 aryl, and 5 to 10 membered heteroaryl; and
each occurrence of R′ is independently selected from H and C1-C6 alkyl.

71. The compound of claim 70, wherein n is 0.

72. The compound of claim 70, wherein n is 1.

73. The compound of claim 70, wherein n is 2.

74. The compound of claim 70, wherein n is 3.

75. The compound of any one of claims 70-74, wherein R¹ is OH.

76. The compound of any one of claims 70-74, wherein R¹ is OR³.

77. The compound of any one of claims 70-74, wherein R¹ is SR³.

78. The compound of any one of claims 70-74, wherein R¹ is NR³R⁴.

79. The compound of any one of claims 70-74, wherein R¹ is selected from OH, OR³, SR³, and NR³R⁴.

80. The compound of any one of claims 70-74, wherein R¹ is

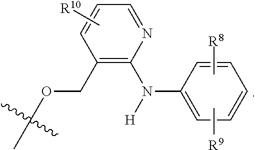

81. The compound of any one of claims 70 and 72-80, wherein R² is hydrogen,

82. The compound of any one of claims 70 and 72-80, wherein R² is OH.

83. The compound of any one of claims 70 and 72-80, wherein R² is OR³.

84. The compound of any one of claims 70 and 72-80, wherein R² is SR³.

85. The compound of any one of claims 70 and 72-80, wherein R² is NR³R⁴.

86. The compound of any one of claims 70-74, wherein R¹ and at least one R² are OH.

87. The compound of any one of claims 70-74, wherein R¹ is 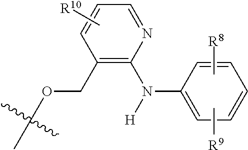
and each occurrence of R² is selected from OH and OR³.

88. The compound of any one of claims 70-85 and 87, wherein R³ is hydrogen.

89. The compound of any one of claims 70-85 and 87, wherein R³ is C1-C10 alkyl optionally substituted with 1-6 halogens.

90. The compound of claim 89, wherein R³ is methyl.

91. The compound of claim 89, wherein R³ is trifluoromethyl.

92. The compound of any one of claims 70-85 and 87, wherein R³ is C6-C10 aryl.

93. The compound of any one of claims 70-85 and 87, wherein R³ is 5 to 10 membered heteroaryl.

94. The compound of any one of claims 70-85 and 88-93, wherein R⁴ is hydrogen.

95. The compound of any one of claims 70-85 and 88-93, wherein R⁴ is C1-C10 alkyl optionally substituted with 1-6 halogens.

96. The compound of claim 85, wherein R⁴ is methyl.

97. The compound of claim 85, wherein R⁴ is trifluoromethyl.

98. The compound of any one of claims 70-85 and 88-93, wherein R⁴ is C6-C10 aryl.

99. The compound of any one of claims 70-85 and 88-93, wherein R⁴ is 5 to 10 membered heteroaryl.

100. The compound of any one of claims 70-85, wherein R³ and R⁴, together with the nitrogen atom to which they are attached, come together to form a 3 to 7 membered heterocyclyl.

101. The compound of any one of claims 70-85, wherein R³ and R⁴, together with the nitrogen atom to which they are attached, come together to form a 5 to 10 membered heteroaryl.

102. The compound of any one of claims 1-101, wherein R⁵ is hydrogen.

103. The compound of any one of claims 1-101, wherein R⁵ is halogen.

104. The compound of claim 103, wherein R⁵ is fluoro or chloro.

105. The compound of any one of claims 1-101, wherein R⁵ is pseudohalogen.

106. The compound of any one of claims 1-101, wherein R⁵ is C1-C10 alkyl optionally substituted with 1-6 halogens.

107. The compound of claim 106, wherein R⁵ is methyl.

108. The compound of claim 106, wherein R⁵ is trifluoromethyl.

109. The compound of any one of claims 1-101, wherein R⁵ is C1-C6 alkoxy.

110. The compound of any one of claims 1-101, wherein R⁵ is cyano.

111. The compound of any one of claims 1-101, wherein R⁵ is nitro.

112. The compound of any one of claims 1-101, wherein R⁵ is C(O)C1-C6 alkyl.

113. The compound of any one of claims 1-101, wherein R⁵ is CO₂C1-C6 alkyl.

114. The compound of any one of claims 1-101, wherein R⁵ is C(O)NHC1-C6 alkyl.

115. The compound of any one of claims 1-101, wherein R⁵ is N(R′)₂.

116. The compound of any one of claims 1-101, wherein R⁵ is C6-C10 aryl.

117. The compound of any one of claims 1-101, wherein R⁵ is 5 to 10 membered heteroaryl.

118. The compound of any one of claims 1-117, wherein R⁶ is halogen.

119. The compound of claim 118, wherein R⁶ is fluoro or chloro.

120. The compound of claim 118, wherein R⁶ is chloro.

121. The compound of any one of claims 1-117, wherein R⁶ is pseudohalogen.

122. The compound of any one of claims 1-117, wherein R⁶ is C1-C10 alkyl optionally substituted with 1-6 halogens.

123. The compound of claim 122, wherein R⁶ is methyl.

124. The compound of claim 122, wherein R⁶ is trifluoromethyl.

125. The compound of any one of claims 1-117, wherein R⁶ is C1-C6 alkoxy.

126. The compound of any one of claims 1-117, wherein R⁶ is cyano.

127. The compound of any one of claims 1-117, wherein R⁶ is nitro.

128. The compound of any one of claims 1-117, wherein R⁶ is C(O)C1-C6 alkyl.

129. The compound of any one of claims 1-117, wherein R⁶ is CO₂C1-C6 alkyl.

130. The compound of any one of claims 1-117, wherein R⁶ is C(O)NHC1-C6 alkyl.

131. The compound of any one of claims 1-117, wherein R⁶ is N(R′)₂.

132. The compound of any one of claims 1-117, wherein R⁶ is C6-C10 aryl.

133. The compound of any one of claims 1-117, wherein R⁶ is 5 to 10 membered heteroaryl.

134. The compound of any one of claims 1-133, wherein R⁷ is halogen.

135. The compound of claim 134, wherein R⁷ is fluoro or chloro.

136. The compound of claim 134, wherein R⁷ is chloro.

137. The compound of any one of claims 1-133, wherein R⁷ is pseudohalogen.

138. The compound of any one of claims 1-133, wherein R⁷ is C1-C10 alkyl optionally substituted with 1-6 halogens.

139. The compound of claim 138, wherein R⁷ is methyl.

140. The compound of claim 138, wherein R⁷ is trifluoromethyl.

141. The compound of any one of claims 1-133, wherein R⁷ is C1-C6 alkoxy.

142. The compound of any one of claims 1-133, wherein R⁷ is cyano.

143. The compound of any one of claims 1-133, wherein R⁷ is nitro.

144. The compound of any one of claims 1-133, wherein R⁷ is C(O)C1-C6 alkyl.

145. The compound of any one of claims 1-133, wherein R⁷ is CO₂C1-C6 alkyl.

146. The compound of any one of claims 1-133, wherein R⁷ is C(O)NHC1-C6 alkyl.

147. The compound of any one of claims 1-133, wherein R⁷ is N(R′)₂.

148. The compound of any one of claims 1-133, wherein R⁷ is C6-C10 aryl.

149. The compound of any one of claims 1-133, wherein R⁷ is 5 to 10 membered heteroaryl.

150. The compound of any one of claims 1-53, 56-74, 80-85, and 87-149, wherein R⁸ is halogen.

151. The compound of claim 150, wherein R⁸ is fluoro or chloro.

152. The compound of claim 150, wherein R⁸ is chloro.

153. The compound of any one of claims 1-53, 56-74, 80-85, and 87-149, wherein R⁸ is pseudohalogen.

154. The compound of any one of claims 1-53, 56-74, 80-85, and 87-149, wherein R⁸ is C1-C10 alkyl optionally substituted with 1-6 halogens.

155. The compound of claim 154, wherein R⁸ is methyl.

156. The compound of claim 154, wherein R⁸ is trifluoromethyl.

157. The compound of any one of claims 1-53, 56-74, 80-85, and 87-149, wherein R⁸ is C1-C6 alkoxy.

158. The compound of any one of claims 1-53, 56-74, 80-85, and 87-149, wherein R⁸ is cyano.

159. The compound of any one of claims 1-53, 56-74, 80-85, and 87-149, wherein R⁸ is nitro.

160. The compound of any one of claims 1-53, 56-74, 80-85, and 87-149, wherein R⁸ is C(O)C1-C6 alkyl.

161. The compound of any one of claims 1-53, 56-74, 80-85, and 87-149, wherein R⁸ is CO₂C1-C6 alkyl.

162. The compound of any one of claims 1-53, 56-74, 80-85, and 87-149, wherein R⁸ is C(O)NHC1-C6 alkyl.

163. The compound of any one of claims 1-53, 56-74, 80-85, and 87-149, wherein R⁸ is N(R′)₂.

164. The compound of any one of claims 1-53, 56-74, 80-85, and 87-149, wherein R⁸ is C6-C10 aryl.

165. The compound of any one of claims 1-53, 56-74, 80-85, and 87-149, wherein R⁸ is 5 to 10 membered heteroaryl.

166. The compound of any one of claims 1-53, 56-74, 80-85, and 87-165, wherein R⁹ is halogen.

167. The compound of claim 166, wherein R⁹ is fluoro or chloro.

168. The compound of claim 166, wherein R⁹ is chloro.

169. The compound of any one of claims 1-53, 56-74, 80-85, and 87-165, wherein R⁹ is pseudohalogen.

170. The compound of any one of claims 1-53, 56-74, 80-85, and 87-165, wherein R⁹ is C1-C10 alkyl optionally substituted with 1-6 halogens.

171. The compound of claim 170, wherein R⁹ is methyl.

172. The compound of claim 170, wherein R⁹ is trifluoromethyl.

173. The compound of any one of claims 1-53, 56-74, 80-85, and 87-165, wherein R⁹ is C1-C6 alkoxy.

174. The compound of any one of claims 1-53, 56-74, 80-85, and 87-165, wherein R⁹ is cyano.

175. The compound of any one of claims 1-53, 56-74, 80-85, and 87-165, wherein R⁹ is nitro.

176. The compound of any one of claims 1-53, 56-74, 80-85, and 87-165, wherein R⁹ is C(O)C1-C6 alkyl.

177. The compound of any one of claims 1-53, 56-74, 80-85, and 87-165, wherein R⁹ is CO₂C1-C6 alkyl.

178. The compound of any one of claims 1-53, 56-74, 80-85, and 87-165, wherein R⁹ is C(O)NHC1-C6 alkyl.

179. The compound of any one of claims 1-53, 56-74, 80-85, and 87-165, wherein R⁹ is N(R′)₂.

180. The compound of any one of claims 1-53, 56-74, 80-85, and 87-165, wherein R⁹ is C6-C10 aryl.

181. The compound of any one of claims 1-53, 56-74, 80-85, and 87-165, wherein R⁹ is 5 to 10 membered heteroaryl.

182. The compound of any one of claims 1-53, 56-74, 80-85, and 87-181, wherein R¹⁰ is halogen.

183. The compound of claim 182, wherein R¹⁰ is fluoro or chloro.

184. The compound of any one of claims 1-53, 56-74, 80-85, and 87-181, wherein R¹⁰ is pseudohalogen.

185. The compound of any one of claims 1-53, 56-74, 80-85, and 87-181, wherein R¹⁰ is C1-C10 alkyl optionally substituted with 1-6 halogens.

186. The compound of claim 185, wherein R¹⁰ is methyl.

187. The compound of claim 185, wherein R¹⁰ is trifluoromethyl.

188. The compound of any one of claims 1-53, 56-74, 80-85, and 87-181, wherein R¹⁰ is C1-C6 alkoxy.

189. The compound of any one of claims 1-53, 56-74, 80-85, and 87-181, wherein R¹⁰ is cyano.

190. The compound of any one of claims 1-53, 56-74, 80-85, and 87-181, wherein R¹⁰ is nitro.

191. The compound of any one of claims 1-53, 56-74, 80-85, and 87-181, wherein R¹⁰ is C(O)C1-C6 alkyl.

192. The compound of any one of claims 1-53, 56-74, 80-85, and 87-181, wherein R¹⁰ is CO₂C1-C6 alkyl.

193. The compound of any one of claims 1-53, 56-74, 80-85, and 87-181, wherein R¹⁰ is C(O)NHC1-C6 alkyl.

194. The compound of any one of claims 1-53, 56-74, 80-85, and 87-181, wherein R¹⁰ is N(R′)₂.

195. The compound of any one of claims 1-194, wherein each R′ is H.

196. The compound of any one of claims 1-194, wherein one R′ is H and the other R′ is C1-C6 alkyl.

197. The compound of any one of claims 1-194, wherein each R′ is C1-C6 alkyl.

198. The compound of any one of claims 1-53, 56-74, 80-85, and 87-181, wherein R¹⁰ is C6-C10 aryl.

199. The compound of any one of claims 1-53, 56-74, 80-85, and 87-181, wherein R¹⁰ is 5 to 10 membered heteroaryl.

200. A compound selected from the group consisting of:

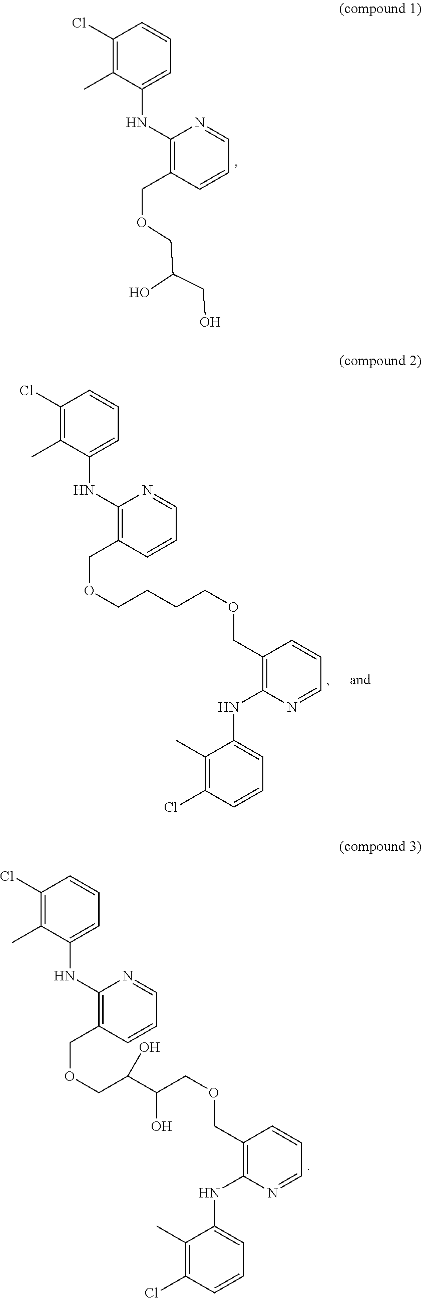 [SMILES: Cc1c(Cl)cccc1Nc1ncccc1COCC(O)CO ; Cc1c(Cl)cccc1Nc1ncccc1COCCCCOCc1cccnc1Nc1cccc(Cl)c1C ; Cc1c(Cl)cccc1Nc1ncccc1COCC(O)C(O)COCc1cccnc1Nc1cccc(Cl)c1C]

201. The compound of any one of claims 1-200, or a pharmaceutically acceptable salt thereof, wherein the compound is a STING agonist.

202. The compound of any one of claims 1-200, or a pharmaceutically acceptable salt thereof, wherein the compound is a STING partial agonist.

203. The compound of any one of claims 1-200, or a pharmaceutically acceptable salt thereof, wherein the compound is a STING antagonist.

204. A pharmaceutical composition comprising a compound of any of claims 1-203, or a pharmaceutically acceptable salt thereof, and at least one excipient.

205. A method of treating cancer in a subject in need thereof, comprising administering to the subject a therapeutically effective amount of a compound of any of claims 1-204, or a pharmaceutically acceptable salt thereof.

206. The method of claim 205, wherein the cancer is a solid tumor.

207. The method of claim 205, wherein the cancer is a blood cancer.

208. A method of treating an autoimmune disorder in a subject in need thereof, comprising administering to the subject a therapeutically effective amount of a compound of any of claims 1-204, or a pharmaceutically acceptable salt thereof.

209. The method of claim 208, wherein the autoimmune disorder is systemic lupus erythematosus (SLE).

210. A method of treating SARS-CoV-2 infection in a subject in need thereof, comprising administering to the subject a therapeutically effective amount of a compound of any of claims 1-204, or a pharmaceutically acceptable salt thereof.

211. The method of claim 210, wherein treating the subject comprises reducing the production of cytokines in the subject.

212. A method of treating hypercytokinemia in a subject in need thereof, comprising administering to the subject a therapeutically effective amount of a compound of any of claims 1-204, or a pharmaceutically acceptable salt thereof.
